SEQUENCE LISTING (38 sequences)

SEQ ID NO: 1 - DNA, 20 bp, synthetic construct
  source 1..20: mol_type = other DNA
ccagaaggtcggatctttcg

SEQ ID NO: 2 - DNA, 23 bp, synthetic construct
  source 1..23: mol_type = other DNA
tgtatccatcgtcagtctttgtg

SEQ ID NO: 3 - DNA, 20 bp, synthetic construct
  source 1..20: mol_type = other DNA
gagaagtttggagaggctgg

SEQ ID NO: 4 - DNA, 20 bp, synthetic construct
  source 1..20: mol_type = other DNA
gctcctccataacgacagag

SEQ ID NO: 5 - DNA, 21 bp, synthetic construct
  source 1..21: mol_type = other DNA
gagaagaaggagactggagag

SEQ ID NO: 6 - DNA, 20 bp, synthetic construct
  source 1..20: mol_type = other DNA
gtcgttttcctcgggattgg

SEQ ID NO: 7 - protein, 421 aa, unidentified
  source 1..421: note = Yarrowia lipolytica
MLSIRSITRSLPIGSRICQQSAMKASTVRPLALRAYSTRPPVTHFSEEEEMFRDMVSKFADEVIAPKVREMDEAEQMDKTIIQDMFDNGLMGIETPEEFGGAGANFTSAIIVVEELAKVDPSVSVMNDVHNTLVNTCIRSWGSDALRNKYLPQLAAQKVGSFALSEPSSGSDAFAMKSRATKTDDGYILNGSKMWITNAAEAELFIVFANLDPSKGYKGITAFVVEKDMGVQIAKKEQKLGIRASSTCVLNFEDVFIPKENLLGEEGKGYKIAIECLNEGRIGIAAQMLGLAGGAFKKATGYAFNDRKQFGQYIGEFQGMQHQIGQAATEIEAARLLVYNAARLKEAGVPFTKEAAMAKLYASQVAGNVASKAVEWMGGVGFTREETLEKFFRDSKIGAIYEGTSNIQLQTIAKIIQKESA

SEQ ID NO: 8 - protein, 346 aa, unidentified
  source 1..346: note = Yarrowia lipolytica
MRSLYINVPGLFPSTSLARETVHHRTEMLRTIRSSSRLGVRAMSTAATRRAAQIGFHTRVPTVVTKAPTLRMQTTPFSSSAPAQTFGDKKYEHILTSTPVPKVALVTLNRPKALNALCTPLIKELNEALQAADADPTIGAIVLTGSEKSFAAGADIKEMKDKTVTSVLNENFIEEWGNMANIKKPIIAAVNGFALGGGCELAMMADIIYAGAKAKFGQPEIKLGVIPGAGGTQRLTRAIGLYRANHYILTGEMFTAQQAADWGLAAKVYEPAQLVDESVKAAAQIASYGQLAVQAAKASVHQSAEVGLRAGLEFERVRFHGLFGTHDQKEGMAAFAEKREPNFKNE

SEQ ID NO: 9 - protein, 388 aa, unidentified
  source 1..388: note = Yarrowia lipolytica
MLRNNLRSLSRAFSTSSMRLGAGMDASKLQITKTKSPKEKQAPKDLIFGHTFTDHMLTVEWTAKDGWAAPQITPYGPLELDPSAVVLHYAFECFEGLKAYKDESGNVRLFRVDKNMHRMNTSAERICLPEFDGAEAAKLIGQLAKLDSAWIPEGRGYSMYLRPSLIGTTAALGVGTPDKALFYVIASPVGPYYPTGFKAVKLEATDYAVRAWPGGVGNKKLGANYAPCIKPQQQAASRGYQQNLWLFGDEGNITEVGTMNAFFVFERNGKKELVTAPLDGTILEGVTRDSILELARERLPSADWIVSERYCTIKEVAEAAEKGELVEAFGAGTAAVVSPIKEIGWGEKTINIPLQPGKEAGKLTETVNEWIGDIQYGKDEYKGWSKVV

SEQ ID NO: 10 - protein, 373 aa, unidentified
  source 1..373: note = Yarrowia lipolytica
MTKLSPLKADRLEITLNKSPKQFDSPKDLAPNSSTDYILQVDYTDEAGWDAPKIGPFGPLQIDPTASVLHYAVECFEGLKLYKNKAGNKLILFRPELNMARLNDSCERIGLPRFDEAELLKCLIEYVKLEGRFVAPGGFIYLRPTAIGTNGGLGLKAPTAAKVFVCATMMQAWNAEPLKLYCSDPKDAVRAWPGGFGYAKLGANYGPTLQENSKAVGAGYHQVLWLYDEDKKTVTEAGGSNFFVAIKKKDSDEIELMTCPISTKLILPGISRRSALEYLGEKHKGSNVTVHEREFGIDEIAEAAKENRLVEAFAIGTAFFVAPVQLIRTPEGELINVPLSQGDSGDFAADVKAKLTSIMYGEEENDWATTVDC

SEQ ID NO: 11 - protein, 463 aa, unidentified
  source 1..463: note = Yarrowia lipolytica
MLRAVSNHACLRQALSSTRTLRTGLQKHVHKTQFVSVRHVSTTKPTERAEVEKPIVQNGELVFPGALRTSFVNNMTFVDPTTQDSMPTYRVVGPDGVQIDKSYKIDLPVDTILKMYKDMVTVSIMDAIMFDAQRQGRLSFYMVSAGEEGMAVGSAAALKPQDHVYSQYREQGAYMYRGFTLDDFMNQLYGNKHDQGKGRNMPVHYGSRELNMHTISSPLATQLPHAAGTAYAQKMAGVDGVTLCYMGEGAASEGDFHAALNIAATRNCPVIYFCRNNGYAISTSAIEQYKGDGIASRAIGYGIETIRVDGNDIFAVHRATKKAREIALRDQKPVLIEGMSYRVSHHSTSDDSFAYRSRGEVESWQRKDNPIVRLRKWLELNKHWNEEEEQKFRTQARKDILTAFSKAEKVKKPEIVSAFTDCWEDIPPHLKEQQQELGEILDTYPEHFDLSAFEGGRGGLHDK

SEQ ID NO: 12 - protein, 398 aa, unidentified
  source 1..398: note = Yarrowia lipolytica
MLTRLRPVLRGGVKASLPSLTKTVSPLRGYSSLVASHDSPNLVNLSEAPFLLNSANAALSHPELAGTAAAEGQAKQLNLYQSVNDALKTALETDETAVLFGEDVAFGGVFRCSMDLQERFGADRVFNTPLTEQGLVGFGIGYAAYGSTAIAEVQFADYVFPAFDQIVNEAAKYRARSSSNFDAGGLTIRMPCGVVGHGAMYHSQSGEAFFSHSPGIKVVMPRSPFQAKGLLLASIRSKDPIIFMEPKILYRASAEYVPTEDYELPIGKADVMQEGSDVTIVGYGTQLYHIHAAAKMAEQKLGASVEIIDLRTISPWDRDTVFESVKKTGRCVVTHEAPRTGGIGAEVAAEVQEKCFLHLESPVQRVTGWDTHMSLAFEDLQVPNVTRIFHSIKKAIEY

SEQ ID NO: 13 - protein, 466 aa, unidentified
  source 1..466: note = Yarrowia lipolytica
MLNQMAFRTARVGGGLRTSFLQRRALHACHIARAVIPFKLADIGEGIKECEVIQWFVEPGARINEFDQICEVQSDKASVEITSRYTGVIKKLHYDAGDMALVGKPLVDIDTGEGGEGASEVAAESSDAAPSTAAATPATPLTASASVASSTATTVSSDPSKAYQKALATPAVRRLTRELGIDIASIKGSGKGGRVMKEDVLSYQKGGSAVSDSATGSVTAATTTSTAGSRLVPLTPTQMGMFKTMTNSLSIPHFLYTDEVCLDKLMELRASVNSLLAKSPSNGVSKISYMPFFIKALSLALKDYPMVNAKVDLSGDKPAVLMRDYHNISIAMDTPNGLLVPTIKNVQDKTIVEIAADLQRLQELGMAGKLSRDDLTGGTISISNIGNVGGTYLSPVIVSEQVAIVGLGKARKLPRYNSQGDIVPEQIINASWSGDHRVLDGMTMALMADKWKAYVVDPKAMLLQLR

SEQ ID NO: 14 - protein, 499 aa, unidentified
  source 1..499: note = Yarrowia lipolytica
MLRTTRILPSKFGVAARTRAVTQFQRCYSSANEELDVLVIGGGPGGYVAAIKAAQAGLKTGCIEKRGSLGGTCLNVGCIPSKSLLNNSQMYHAIKTDSANRGIEVSDVKMNIAKLQEAKETSVKGLTGGIEMLFKKNKVNYYKGAGSFVSDSEVKVDPIDGGEAVTLKAKNIIIATGSEPTPFPGITIDEKKIVSSTGALALEAVPKKMVIIGGGIIGLEMGSVWSRLGSEVTVVEFQNAIGAGMDDEIAKAAQKMLTKQGIKFKLGTKVLSGAIEGDGVKVEVENVKKGDKETLDADVLLVAIGRRPYSEGLNLEAAGVEKDDKGRIIIDQEYRTNKSNIRCIGDVTFGPMLAHKAEEEGVATAEYIATGHGHVNYAAIPSVMYTHPEVAWVGQTEQQVKEAGIKYNVGKFPFAANSRAKTNLDTEGTVKFIADKETDRILGIHIIGPNAGEMIAEGVLALEYGASCEDIARTCHAHPTLSEAFKEAAMATYDKAIHF

SEQ ID NO: 15 - protein, 600 aa, unidentified
  source 1..600: note = Yarrowia lipolytica
MTATSATASTKKDGNVHLGEYLFSRIKQLGIDNILGVPGDFNLHLLDYIYRVPDLNWVGCCNELNAAYAADGYGRVKHLPGVLVTTYGVGELSALNGVSGAFAEQAPLLHIVGTTGRPIQEESWLVHHVLPKENSLQEPDHLVYQGMSEKVRCCSAFLNDKETAAAEIDRVLTTICQKSLPGYLFIPVDMTWQPLDAARLDSKLDYSVKNEDPKLENEIVDSILSEIYASKNATVFADVLTARHRATDLVRELAKKTNFPNYTSPLGKGILNENEDRFVGVYNGQLSLEGVAKSIEASDCVLFTGPLLSDSNTGGFSHNLKDENTIYLSPDYVKVKGKLYEGVHFFPVLKKVVEQLDVEKISAESKTAAMPTISPPPVIDSPLPISQTFLVDSMSKMMRPDDLLIVETGTFQFACSDVKFNTNNSLLTQIFYSCIGFTLPATLGAALAKREDAAAKNDKGRVILVEGDGSAQMTIQELGTMVRQGLNPIIFLLNNDGYSIERAIHGPEQSYNDICPKWKWTKLLEAFGGTEGVDSFSTTVKTREELEALIGDEKFMNSDKAQLVEVIMDVNDYPWRLNEQIKLMGGKNMKVFAEYQKKNP

SEQ ID NO: 16 - protein, 677 aa, unidentified
  source 1..677: note = Yarrowia lipolytica
MAKERGKTQFTVRDVTNFLNGGEEETQIVEKIMSSIERDPVLSVTADYDCNLQQARKQTMERVAALSPYLVTDTEKLSLWRAQLHGMVDMSTRTRLSIHNNLFIGSIRGSGTPEQFKYWVKKGAVAVKQFYGCFAMTELGHGSNLKGLETTATYDQDSDQFIINTPHIGATKWWIGGAAHTSTHCVCFAKLIVHGKDYGTRNFVVPLRNVHDHSLKVGVSIGDIGKKMGRDGVDNGWIQFTNVRIPRQNMLMRYAKVSDTGVVTKPALDQLTYGALIRGRVSMIADSFHVSKRFLTIALRYACVRRQFGTSGDTKETKIIDYPYHQRRLLPLLAYCYAMKMGADEAQKTWIETTDRILALNPNDPAQKNDLEKAVTDTKELFAASAGMKAFTTWGCAKIIDECRQACGGHGYSGYNGFGQGYADWVVQCTWEGDNNVLCLSMGRGLVQSALQILAGKHVGASIQYVGDKSKISQNGQGTPREQLLSPEFLVEAFRTASRNNILRTTDKYQELVKTLNPDQAFEELSQQRFQCARIHTRQHLISSFYARIATAKDDIKPHLLKLANLFALWSIEEDTGIFLRENILTPGDIDLINSLVDELCVAVRDQVIGLTDAFGLSDFFINAPIGSYDGNVYEKYFAKVNQQNPATNPRPPYYESTLKPFLFREEEDDEICDLDE

SEQ ID NO: 17 - protein, 700 aa, unidentified
  source 1..700: note = Yarrowia lipolytica
MNPNNTGTIEINGKEYNTFTEPPVAMAQERAKTSFPVREMTYFLDGGEKNTLKNEQIMEEIERDPLFNNDNYYDLNKEQIRELTMERVAKLSLFVRDQPEDDIKKRFALIGIADMGTYTRLGVHYGLFFGAVRGTGTAEQFGHWISKGAGDLRKFYGCFSMTELGHGSNLAGLETTAIYDEETDEFIINTPHIAATKWWIGGAAHTATHTVVFARLIVKGKDYGVKTFVVQLRNINDHSLKVGISIGDIGKKMGRDGIDNGWIQFTNVRIPRQNLLMKYTKVDREGNVTQPPLAQLTYGSLITGRVSMASDSHQVGKRFITIALRYACIRRQFSTTPGQPETKIIDYPYHQRRLLPLLAYVYALKMTADEVGALFSRTMLKMDDLKPDDKAGLNEVVSDVKELFSVSAGLKAFSTWACADVIDKTRQACGGHGYSGYNGFGQAYADWVVQCTWEGDNNILTLSAGRALIQSAVALRKGEPVGNAVSYLKRYKDLANAKLNGRSLTDPKVLVEAWEVAAGNIINRATDQYEKLIGEGLNADQAFEVLSQQRFQAAKVHTRRHLIAAFFSRIDTEAGEAIKQPLLNLALLFALWSIEEDSGLFLREGFLEPKDIDTVTELVNKYCTTVREEVIGYTDAFNLSDYFINAPIGCYDGDAYRHYFQKVNEQNPARDPRPPYYASTLKPFLFREEEDDDICELDEE

SEQ ID NO: 18 - protein, 700 aa, unidentified
  source 1..700: note = Yarrowia lipolytica
MISPNLTANVEIDGKQYNTFTEPPKALAGERAKVKFPIKDMTEFLHGGEENVTMIERLMTELERDPVLNVSGDYDMPKEQLRETAVARIAALSGHWKKDTEKEALLRSQLHGIVDMGTRIRLGVHTGLFMGAIRGSGTKEQYDYWVRKGAADVKGFYGCFAMTELGHGSNVAGLETTATYIQDTDEFIINTPNTGATKWWIGGAAHSATHTACFARLLVDGKDYGVKIFVVQLRDVSSHSLMPGIALGDIGKKMGRDAIDNGWIQFTNVRIPRQNMLMKYAKVSSTGKVSQPPLAQLTYGALIGGRVTMIADSFFVSQRFITIALRYACVRRQFGTTPGQPETKIIDYPYHQRRLLPLLAFTYAMKMAADQSQIQYDQTTDLLQTIDPKDKGALGKAIVDLKELFASSAGLKAFTTWTCANIIDQCRQACGGHGYSGYNGFGQAYADWVVQCTWEGDNNVLCLSMGRGLIQSCLGHRKGKPLGSSVGYLANKGLEQATLSGRDLKDPKVLIEAWEKVANGAIQRATDKFVELTKGGLSPDQAFEELSQQRFQCAKIHTRKHLVTAFYERINASAKADVKPYLINLANLFTLWSIEEDSGLFLREGFLQPKDIDQVTELVNHYCKEVRDQVAGYTDAFGLSDWFINAPIGNYDGDVYKHYFAKVNQQNPAQNPRPPYYESTLRPFLFREDEDDDICELDEE

SEQ ID NO: 19 - protein, 701 aa, unidentified
  source 1..701: note = Yarrowia lipolytica
MITPNPANDIVHDGKLYDTFTEPPKLMAQERAQLDFDPRDITYFLDGSKEETELLESLMLMYERDPLFNNQNEYDESFETLRERSVKRIFQLSKSIAMDPEPMSFRKIGFLGILDMGTYARLGVHYALFCNSIRGQGTPDQLMYWLDQGAMVIKGFYGCFAMTEMGHGSNLSRLETIATFDKETDEFIINTPHVGATKWWIGGAAHTATHTLAFARLQVDGKDYGVKSFVVPLRNLDDHSLRPGIATGDIGKKMGRDAVDNGWIQFTNVRVPRNYMLMKHTKVLRDGTVKQPPLAQLTYGSLITGRVQMTTDSHNVSKKFLTIALRYATIRRQFSSTPGEPETRLIDYLYHQRRLLPLMAYSYAMKLAGDHVRELFFASQEKAESLKEDDKAGVESYVQDIKELFSVSAGLKAATTWACADIIDKARQACGGHGYSAYNGFGQAFQDWVVQCTWEGDNTVLTLSAGRALIQSALVYRKEGKLGNATKYLSRSKELANAKRNGRSLEDPKLLVEAWEAVSAGAINAATDAYEELSKQGVSVDECFEQVSQERFQAARIHTRRALIEAFYSRIATADEKVKPHLIPLANLFALWSIEEDSALFLAEGYFEPEDIIEVTSLVNKYCGIVRKNVIGYTDAFNLSDYFINAAIGRYDGDVYKNYFEKVKQQYPPEGGKPHYYEDVMKPFLHRERIPDVPMEPEDIQ

SEQ ID NO: 20 - protein, 699 aa, unidentified
  source 1..699: note = Yarrowia lipolytica
MNNNPTNVILGGKEYDTFTEPPAQMELERAKTQFKVRDVTNFLTGSEQETLLTERIMREIERDPVLNVAGDYDADLPTKRRQAVERIGALARYLPKDSEKEAILRGQLHGIVDMGTRTRIAVHYGLFMGAIRGSGTKEQYDYWVAKGAATLHKFYGCFAMTELGHGSNVAGLETTATLDKDTDEFIINTPNSGATKWWIGGAAHSATHTACLARLIVDGKDYGVKIFIVQLRDLNSHSLLNGIAIGDIGKKMGRDAIDNGWIQFTDVRIPRQNMLMRYDRVSRDGEVTTSELAQLTYGALLSGRVTMIAESHLLSARFLTIALRYACIRRQFGAVPDKPETKLIDYPYHQRRLLPLLAYTYAMKMGADEAQQQYNSSFGALLKLNPVKDAEKFAVATADLKALFASSAGMKAFTTWAAAKIIDECRQACGGHGYSGYNGFGQAYADWVVQCTWEGDNNVLCLSMGRSLIQSCIAMRKKKGHVGKSVEYLQRRDELQNARVDNKPLTDPAVLITAWEKVACEAINRATDSFIKLTQEGLSPDQAFEELSQQRFECARIHTRKHLITSFYARISKAKARVKPHLTVLANLFAVWSIEEDSGLFLREGCFEPAEMDEITALVDELCCEAREQVIGFTDAFNLSDFFINAPIGRFDGDAYKHYMDEVKAANNPRNTHAPYYETKLRPFLFRPDEDEEICDLDE

SEQ ID NO: 21 - protein, 689 aa, unidentified
  source 1..689: note = Yarrowia lipolytica
MLSQQSLNTFTEPPVEMARERNQTSFNPRLLTYFLDGGEKNTLLMDRLMQEYERDPVFRNEGDYDITDVAQSRELAFKRIAKLIEYVHTDDEETYLYRCMLLGQIDMGAFARYAIHHGVWGGAIRGAGTPEQYEFWVKKGSLSVKKFYGSFSMTELGHGSNLVGLETTATLDKNADEFVINTPNVAATKWWIGGAADTATHTAVFARLIVDGEDHGVKTFVVQLRDVETHNLMPGIAIGDCGKKMGRQGTDNGWIQFTHVRIPRQNMLMRYCHVDSDGNVTEPMMAQMAYGALLAGRVGMAMDSYFTSRKFLTIALRYATIRRAFAAGGGQETKLIDYPYHQRRLLPLMAQTYAIKCTADKVRDQFVKVTDMLLNLDVSDQEAVPKAIAEAKELFSVSAGVKATTTWACAHTIDQCRQACGGHGYSAYNGFGRAYSDWVIQCTWEGDNNILCLSAGRALVQSNRAVRAGKPIGGPTAYLAAPAGSPKLAGRNLYDPKVMIGAWETVSRALINRTTDEFEVLAKKGLSTAQAYEELSQQRFLCTRIHTRLYMVKNFYERIAEEGTEFTKEPLTRLANLYAFWSVEEEAGIFLREGYITPQELKYISAEIRKQLLEVRKDVIGYTDAFNVPDFFLNSAIGRADGDVYKNYFKVVNTQNPPQDPRPPYYESVIRPFLFRKDEDEEICSLEDE

SEQ ID NO: 22 - protein, 530 aa, unidentified
  source 1..530: note = Yarrowia lipolytica
MSEQYTPEQVAEHNSPESLWIIIDGNVFDLTEFQKEHPGGKKILKRVAGKDATKFFHKYHDAPKIMRKVGHKFKIGTLKDAEANPTRAMIAPNKTTALEPYGDLVPYADPNWYHGYHNPYYKESHAKLRDEVRQWVEEKIEPFVEEWDEEKEVPKEIFQEMGKRGYLAGSLGTPYKELAKYTNVKPASVPIEEYDMFHELIITDEIMRAGSGGLTWNLLGGYCIGLPPVIKFAKEPLKERILPGLLDGSKRICLCITEPDAGSDVANITTTAEKTPDGKFYIVNGIKKWITNGIWADYFTVAVRTGGPGSGMNGISVLLLERGMEGLETRRMNTQGMLSSGSTWVTMEDVKVPVENLLGKENKGFKVIMTNFNHERVGIIIQANRASRVCYEEACKYAHKRKTFGKPLIEHPVIRAKLANMAIRIESTHAWLENLVFQCQMFPEEEAMLRLGGAIAGCKAQATQTLELCAREASQIFGGLSYTRGGLGGKVERLYREVRAYAIPGGSEEIMLDLAMRQALKVHKAVGAKL

SEQ ID NO: 23 - protein, 417 aa, unidentified
  source 1..417: note = Yarrowia lipolytica
MLTRISRLAPAARGFATSSVNRSTAAMDWQDPFQLDSLLTEDEIAVAEAARDFCQTELYPKVLEGYRTEEFPRSIMKQMGEVGLLGTTVKSHGCPGMSSVAYGLVAREVERVDSGYRSAMSVQSSLVMHPIEQFGSQEQKDRFLPKLASGEMIGCFGLTEPNHGSDPGSMETVAKMHPTKKGVIVLNGAKNWITNSPIADLMVVWAKLDGKIRGFLVERSQVASGLATPAIKNKTALRASITGMIQMDDVEIPVENMFPEVTGLKGPFTCLNSARYGIAWGTMGALSESIKLAREYSLDRKQFKGQPLAKYQLIQKKLADALTDATYGQVAAIQVGRLKDAGNCPPELISMIKRQNCDRALAGARNLMEIFGGNAASDEYHIGRIAANLWVVQTYEGQSDIHALILGRAMTGVQAFA

SEQ ID NO: 24 - protein, 577 aa, unidentified
  source 1..577: note = Yarrowia lipolytica
MATATTGFFQDRPTLLNQYDGNSALKKVAEFYLSHKTLAAVLPELRELGDYVISKEIFEWQEDAETNIPRVEHQGTFGHPKNRLIVSQGWKLLKAKAFAAGIISDSYERKYAEESRVVAFLKNWLFSASSATVLCPFAMTDGTARVLEVFAKDDERLQEWYNHLVSSDPTQAWTSGQWMTERPGGSDVRNTETVATSSYRKEEGAESRPNASANNQIENNATSDIVTVGTGPKDYTVNGFKWFSSATDSECSILLAKREGETGLSCFIGKLTPETVIINRLKKKFGTIALPTAELELRNHPVILLGQANNGVPVIATVLNITRLYSSTSALSCYGRSLHIAKQYALVRSVFGKKLSATPAHLKTLAHEQVQLTGLMFLLFEGISLLGQSEVTGLSPHREAVLRVLPGIAKAYCCKKATIGCSECMEALGGAGYLEHEIDINIARLYRESQVNVIWEGTTNVLSDDVARVMKGKRGPLMIGAFEKYIAEVSSDQSVLDEFAAWKKWVEGMDLEATRADARNVTYKLAHVIVRALLLRNAVKTGDRVDVEIAERWKNADLSGDAEYDSEILYGQKTAKL

SEQ ID NO: 25 - protein, 395 aa, unidentified
  source 1..395: note = Yarrowia lipolytica
MASTTPQSTSSASSSRVGGTMINPKGNFDPTIPAILPHEQMYAIQVGDQRFTLSGASLSSDGPSYFTEFFLKHKDNPPVLFLDRSPDIFKLIVRHLQGYYIMSSDEATSVYLLIDARYYQLPRLTQQLLSSELMIRIGNESIKVPRTAFDTPGDEQNFFTAGMGVFFGLEHDTSDEVHSQFIRPPPLAIPTVPKRDGKLFKELLAIQLGGFVNIHSEEHREALLRECRYFRFRGLEQKMIRHEIRVDPIRKKKEISLFLKNLDVKKIELVDGVVMYKRPFVDTELMDLVFQVPDEAVLSVALSRVSFYGATKIKLDKIAKLHGLTFGAVDLENAHLEISGSQAAVEVQKRGKSDSHTFLTKSVWRFKDGGSGELVLLKGEGYLSEGEFNRTREFF

SEQ ID NO: 26 - protein, 901 aa, unidentified
  source 1..901: note = Yarrowia lipolytica
MSGELRYDGKVVIVTGAGGGLGKAYALFYGSRGASVVVNDLGGDFKGDGAQAGSGKRVADVVVDEIVSKGGKAVANYDSVENGDKIVETAVKAFGSVHIVINNAGILRDISFKKMTDKDWDLVYKVHVFGAYKVTRAAWPYFRKQKYGRVISTSSAAGLYGNFGQTNYSAAKLALVGFGETLAKEGAKYNITSNVIAPLAASRMTETVMPEDILKLLKPEYVVPLVGYLTHDSVTESYGIYEVGAGYMAKIRWERGNGAVFKGDDTFTPSAILKRWDEVTSFESPTYPNGPADFFKYAEESVKRPENPQGPTVSFKDQVVIVTGAGAGIGRAYSHLLAKLGAKVVVNDFGNPQKVVDEIKALGGIAVADKNNVIHGEKVVQTAIDAFGAVHAVVNNAGILRDKSFANMDDEMWQLIFDVHLNGTYSVTKAAWPHFLKQKYGRVINTTSTSGIYGNFGQANYSAAKAGILGFSRALAREGEKYNILVNTIAPNAGTAMTASVFTEEMLELFKPDFIAPITVLLASDQAPVTGDLFETGSAWIGQTRWQRAGGKAFNTKKGVTPEMVRDSWAKIVDFDDGNSTHPTTPSESTTQILENIFNVPDEEVEETALVAGPGGPGILNKEGEPFDYTYTYRDLILYNLGLGAKANELKYVFEGDDDFQTVPTFGVIPYMGGLITTNYGDFVPNFNPMMLLHGEQYLEIRQWPIPTNATLENKAKVIDVVDKGKAALLVTATTTTNKETGEEVFYNESSLFIRGSGGFGGKSTGTDRGAATAANKPPARAPDFVKEIKIQEDQAAIYRLSGDYNPLHIDPAFAAVGNFDRPILHGLCSFGVSGKALYDQFGPFKNAKVRFAGHVFPGETLKVEGWKEGNKVIFQTKVVERGTTAISNAAIELFPKDAKL

SEQ ID NO: 27 - protein, 414 aa, unidentified
  source 1..414: note = Yarrowia lipolytica
MDRLNNLATQLEQNPAKGLDAITSKNPDDVVITAAYRTAHTKGGKGLFKDTSSSELLASLLEGLVKESKIDPKLIGDVVCGNVLAAGAGATEHRAACLVAGIPETVPFVALNRQCSSGLMAVNDVANKIRAGQIDIGIGCGVESMSNQYGPNSVTPFSNKFQNNEEAKKCLIPMGITSENVAAKYNVSRKAQDAFAAKSYEKAAAAQAAGKFDQEILPIKTTVLDDDDNEKEVTVNKDDGIRPGVTAEKLGKLKPAFSAEGTTHAGNASQISDGAGAVLLMRRSVAEKLGQPILAKFVHCKTVGVPPELMGIGPAYAIPAVLEDLGLTVNDVDVFEINEAFASQALFSIQHCGIDESKVNPRGGAIAIGHPLGATGARQFATLLSELKESGKKVGVTSMCIGTGMGAASLVVAE

SEQ ID NO: 28 - protein, 691 aa, unidentified
  source 1..691: note = Yarrowia lipolytica
MVGYTISSKPVSVEVGPAKPGETAPRRNVIAKDAPVVFPDNDSSLTTVYKLFKKYAEINSERKAMGWRDTIDIHVETKQVTKVVDGVEKKVPKEWKYFEMGPYKWLSYKEALKLVHDYGAGLRHLGIKPKEKMHIYAQTSHRWMLSGLASLSQGIPIVTAYDTLGEEGLTRSLQETNSVIMFTDKALLSSLKVSLKKGTDLRIIIYGGDLTPDDKKAGNTEIDAIKEIVPDMKIYTMDEVVALGREHPHPVEEVDYEDLAFIMYTSGSTGVPKGVVLQHKQILASVAGVTKIIDRSIIGNTDRLLNFLPLAHIFEFVFEMVTFWWGASLGYGTVKTISDLSMKNCKGDIRELKPTIMVGVPAVWEPMRKGILGKIKELSPLMQRVFWASFAAKQRLDENGLPGGSILDSLIFKKVKDATGGCLRYVCNGGAPVSVDTQKFITTLICPMLIGCGLTETTANTTIMSPKSYAFGTIGEPTAAVTLKLIDVPEAGYFAENNQGELCIKGNVVMKEYYKNEEETKKAFSDDGYFLTGDIAEWTANGQLRIIDRRKNLVKTQNGEYIALEKLETQYRSSSYVANLCVYADQNRVKPIALVIPNEGPTKKLAQSLGVDSDDWDAVCSNKKVVKAVLKDMLDTGRSLGLSGIELLQGIVLLPGEWTPQNSYLTAAQKLNRKKIVDDNKKEIDECYEQS

SEQ ID NO: 29 - protein, 315 aa, unidentified
  source 1..315: note = Yarrowia lipolytica
MAAISKDYVLSPWGKAVAGAAGAVLANTLVYPLDIVKTRLQVQVKRKEGGPLPAFEEGHFEHYEGTVDALKKIYAANGLAGLYQGLPSCLLGVASTNFAYFYWYGFIRDSYIKRNPGKALSTPIELLLGAVAGALAQVFTIPVAVITTRQQTSDAKSRQGFLATAKSVVDDDGISGLWRGLKASLVLVINPSITYGSFERLRTILFKGKLHLSPGENFLLGALSKAMATIATQPMIVAKVMQQSKTKGGKQFNSFVQALVFLFKEEGILGMWKGVGPQISKGIIVQGLLFMIKDQVELFIVLLFRLMKAPTLIKG

SEQ ID NO: 30 - protein, 747 aa, unidentified
  source 1..747: note = Yarrowia lipolytica
MSDSTFAHQESLPKLPIPDLKDTCDNYLEALRPLQTAHEHERTERAVHEFLNTSGPILQEELKQYGKPRSSYIEQFWYDSYLNYDSPLVLNLNPFFLIEDDPTPVQQSQVDRAATLTTSSLRFIRALRREELPPDNVRGTKLCMYQYSRLFASARIPSANGCVMQSDSTSKHIVVLCRSQFYWFDVLDDNNDLIMVEKDIALNFETIIQDAEGTPITEIAKGAVGVLTTENRRVWASIRDQMNRATDSSNYECLKLIDSALFVVCLDHAAPESLSDLTRNALCGLSELQKGVQVGTCTNRWYDKLQIIVTQNAKAGVTFEHTGVDGHTVLRFVSDIYTDSILRFANTISGAAPSLWKTVSPDPAKRDPASFGNVSTTPRKLEWSIGPELQTALRFAETRNSDLIHQNEFECLSFNHYGKKAICEMGCSPDAFVQMVFQAAYYVLYGRVECTYEPAMTKTFLHGRTESIRSVTKESSEFCRKFCEDLPAETKLEYLRKACQQHTKNTKKSSQGLGQDRHLYALLCIWRRYLEESDDNDSTTSQTSIGSQTAVDIGDEMTPITGLGDVHLPKAGGSIPSIFSDPGWDKLNNTILSTSNCGNPALKLFGFGPTSPDGYGLGYIIKDDSITICASSKHRQTARFLETLSTVMMEIRLLFREVAKAKAQRPAGASRARSCERVPQVHTTKQVHFGRTLPRSRKPSETEIYDEEMSYLLGGYGYFDMADFENAADTERLGRKEIGRKLRLAEY

SEQ ID NO: 31 - protein, 417 aa, unidentified
  source 1..417: note = Yarrowia lipolytica
MLRSTLSHMQKSVRVASMARQSMAAARLYSTDTNGSGSGIEEIEELRQAAIDFCAAEITPELQEKTDVDNAFPNHLWEKFGEAGFLGMTAPEKYGGLNLGYLAHSVVMEELSRASGSIALSYSAHSQLCINQLSLHTTPEQGEKYLPDLIAGKKIGALAMSEAGAGSDVVSMKTTAKKVDGGYVLNGTKMWITNGPDADYMVVYAKTDPAAGARGITAFIVEKGMDGFSCARKLDKLGMRGSNTGELIFEDVFVPEGNVLGKINGGVYVLMSGLDLERLVLAAGPIGLMHRALDEALPYVHTRTQFGKPIAHNQLLQGKLADMYTAFASTRAFQYQTAKAADLGQHTSKECASVILLAAEQATQVALDAIQCMGGMGYMMETVPQRLLRDAKLYEIGAGTSEVRRMLIGRAFNKDFA

SEQ ID NO: 32 - protein, 270 aa, unidentified
  source 1..270: note = Yarrowia lipolytica
MPAERTLLASFPLPKKGETQPDDKKLLSLYDTEDYYLIEFHSPKDNRLSNVFIDAFVEALHYLLATKEDEPKPLVTTSAIPKFYSNGLDLEAAMKTDRFFERYLGRLQRSLLEFPWPTIAYVNGHAFAGGFILANSHDFKVQNDKKGWLCMNEIEFDAPLIGQLMSIHAHQYGPVIGRKITMMGHRFTAAEALKDGIIDAAGGWDEVEEIVAKVRNSVGKNSYGQMRMTMFAPVLDHCINYYEEEARDVARQAAARDHQAARIEEVKAKL

SEQ ID NO: 33 - protein, 292 aa, unidentified
  source 1..292: note = Yarrowia lipolytica
MLRTISRTRTMVPSRHLISYRFFSDVSTTKGETFTLTKHFLDASNTAHIAVYSLNRPEAMNSISKKLLEEFETYINSLAAEGRHQNVTNTRALILSSELPKVFCAGADLKERKTFTDADTAAFLNKLNGTLDTIQSLHMPTITAIQGFALGGGAEISLATDFRVLSDVAQFGLPETRLAILPGAGGTKRLPKLIGYSRALDLVLTGRRVKADEALHLGIANRTGENALETALEMAKLICEGGPIAINAAKMAVRGQSKEWEIAAYNKVVNSEDKFEALSAFKEKRKPIFKGR

SEQ ID NO: 34 - protein, 262 aa, unidentified
  source 1..262: note = Yarrowia lipolytica
MTVPKLPLSFPLDQAVDTIRLSDKGSYYILELNAPPDNRLTPSFITAFIEALDTLKFYYDPKPLVTTSTSPKFYSNGLDYELAQKTPNFMSLYFYPLLKAFVDFPWPTIANINGHCFAGAFCLAAFHDYRVMNPAKGWLCMNELEFDAPLMGPLMSIYVTKFGAAIAQRISLNAERFTAKTALEAGLIHAHGSWPEVEEIVKRIGEYSNRTFYSQMRAGILRNVLTTMDYDNETRILHERRDRLWKYEDENKPRIGALLSKL

SEQ ID NO: 35 - protein, 473 aa, unidentified
  source 1..473: note = Yarrowia lipolytica
MLRTLARTRMPLRAQINTRFMSQTTAAAEESPVLFSDNGTTRTITLNRPKKLNALDDPMCRAIFPRLKEWLKSDSANVVLFKGSGNKAFCAGGDVATLAKQNAAGAQGIEQSRDFFCTEYAMDFLLSVYPKPVIALNHGITMGGGLGVSMHLPFRVATETTLSAMPETSIGFFCDVGGTFFLPRLDGELGTFLALTSSRLKGYEAVAAGFATHYIPSARLDDLEKAIASLDIPKGRDATELYSQLNALLNDYSEVPANFKFQYGGEFRALIDRTFKYDAIEDIVAALKQEGEAGEKIIKLLGERSPTSVKVTLAALRRGRNQDIQSVFNDECNYAENFMHSREFIEGVTAKLIDKPAREPQWEPSSFEGVTPEIVKSFIQKRPGSKNDEMEVVINDTYKQYPWNFGLPREVEIEQYIKGETRSSDYKASPAEVVNHFTNKYSGKPGVSEKVKEVMQRKTKPDPNEPKVLDWVN

SEQ ID NO: 36 - protein, 265 aa, unidentified
  source 1..265: note = Yarrowia lipolytica
MSLKLPQSFSLGGPDQLVLVTEEPTYYVLKLNSPPDNRLTPELLTAWIEAMEALALYAEEPKPLVITSAMPKYFSNGLDLEATANVDNFTRDFYYPLIKTLLNFPWPTIGFLNGHTFAGAFVVAGFFDFRVMNPDRGYLCMNEIEFDAPIMGGGMVVFTKLYGRAVAQRITMLAERFPAQKALEAGIIHAKGMWPEVEQMVKKVSHVSGKKFYSLIRKEVMKDIIEALQDSDHQLFLDALMGDHIDPKIEEEQRKRLDPKIKALL

SEQ ID NO: 37 - protein, 266 aa, unidentified
  source 1..266: note = Yarrowia lipolytica
MSLLQTFPLPNKGETQTDEQKLMSLYDKGDFYCLEFHSPPDNRLTCTFIDAFVEALNWLLLKKSGAPKPLVTTSAIPKFYSNGLDLEAAIRTPQFFQRYLGRMQRALIEFPWPTIAYVNGHAFAGGFILANSHDFKVQNSKKGWLCMNEIEFDAPLIGQLMSIHAHQYGPVLARKITMMGHRFPAAESLKDGLVDAIGGWPEVEEIVQKCKKYAGKNYYNQMRMTIFAPVLDHCINYYDEEARDIARQEAAQQFIDDRQAEIKAKL

SEQ ID NO: 38 - protein, 277 aa, unidentified
  source 1..277: note = Yarrowia lipolytica
MPLDYSAYDQFKISFPAEWVALVETNRPKSLNSYNLKTWEQFGQIFIQMSDDSDIRCIVFGGEGRAFCSGLDIKEMGSFGADDDDMEAARKAIKFRRFITDFQDNIKQPHLCRKPIIAVCHGISFGLALDMISGMDIRIASKDVKFSIKEADIGLAADMGTLQRLPKVVNNLGWIKELAYTARIFGAEEAFTQGLVTHVVEDKAAGMKKALEMAVLISEKSPVAVQGSKETINYAIDHTIEDGLAQIANFNTYAVAGDTIEAAMSMAMKKKPTYEKL

(19) United States
(12) Patent Application Publication
(10) Pub. No.: US 2026/0209780 A1
(43) Pub. Date: Jul. 23, 2026

(54) PRODUCTION OF CANNABINOIDS AND ENGINEERED CELLS THEREFOR

(71) Applicant: Cellibre, Inc., San Diego, CA (US)

(72) Inventors: Diep Minh Ngoc Nguyen, San Diego, CA (US); Caleb Marshall Walker, San Diego, CA (US); Nicky Christopher Caiazza, Encinitas, CA (US); Jun Urano, Irvine, CA (US)

(21) Appl. No.: 19/142,525

(22) PCT Filed: Dec. 22, 2023
(86) PCT No.: PCT/US2023/085853

§ 371 Date: Jun. 23, 2025

Related U.S. Application Data
(60) Provisional application No. 63/434,892, filed on Dec. 22, 2022.

(51) Int. Cl. C12N 15/52; C12N 9/00; C12N 9/02; C12N 9/10; C12N 9/88; C12P 7/42; C12P 17/06

(52) U.S. Cl. CPC C12N 15/52; C12N 9/0004; C12N 9/001; C12N 9/1029; C12N 9/104; C12N 9/1096; C12N 9/88; C12N 9/93; C12P 7/42; C12P 17/06; C12Y 103/03006; C12Y 103/08004; C12Y 103/08006; C12Y 103/99003; C12Y 203/01007; C12Y 203/01016; C12Y 206/01042; C12Y 602/01003

(57) ABSTRACT

Disclosed herein are novel cells which are engineered to catabolize a reduced number of acyl-CoA and branched chain amino acids and methods for improved cannabinoid production using these cells. Additionally, cells which are engineered to produce rare cannabinoids, methods for producing these novel rare cannabinoids, as well as the novel rare cannabinoids compounds thus produced, are also disclosed.

### RELATED APPLICATION

This application claims priority to, and the benefit of, co-pending U.S. Provisional Application No. 63/434,892, filed Dec. 22, 2022. The disclosure of said provisional application is hereby incorporated by reference in its entirety.

### BACKGROUND OF THE INVENTION

The Cannabaceae family of plants produces numerous different cannabinoids (>=120) in variable, relative quantities over a 7-10 week flowering period. Many of these cannabinoids have been and are currently being explored as therapeutics in chordates (e.g., mammals), and as a result, they are largely approved (>=35 states) for either medical and/or recreational uses in the United States (Abrams DI Eur J Int Med 2018, 49, 7-11). Specifically, the most sought after (phyto)cannabinoids are: tetrahydrocannabinolic acid (THCA), cannabidiolic acid (CBDA), and cannabichromenic acid (CBCA) and related minor cannabinoids. These phytocannabinoids and their associated chemical analogs are all biosynthesized in various quantities from the same precursor: cannabigerolic acid (CBGA). As a result, achieving high titers in the biosynthesis of THC(A), CBD(A) and CBC(A) either in the plant or in a recombinant host organism is of prime importance. Yet, efficient biosynthesis of these cannabinoids in a recombinant host, such as yeast, has been hindered by endogenous pathways which catabolize precursor molecules and prevent their efficient use in the cannabinoid biosynthesis pathway.

Thus, there remains a need to provide an engineered recombinant host that provides increased availability of cannabinoid biosynthesis precursor molecules, including Acyl-CoA molecules, by deleting, inactivating or otherwise mutating genes which influence cellular metabolism of these precursor molecules.

### SUMMARY OF THE INVENTION

Some aspects of the present disclosure are directed to a cell engineered to provide increased production of cannabinoids and/or derivatives thereof compared to a control cell. The engineered cell expresses one or more enzymes of the cannabinoid biosynthesis pathway, and at least one gene encoding one or more enzymes or other proteins influencing metabolism of at least one acyl-CoA molecule of the cell has been deleted, inactivated, or otherwise mutated, thereby permitting the redirection of acyl-CoA molecules from a cellular metabolic pathway to a cannabinoid biosynthesis pathway to achieve increased production of cannabinoids and/or derivatives thereof in the engineered cell compared to a control cell lacking the same deletion, inactivation or other mutation of the at least one gene.

In some embodiments, the cellular metabolic pathway is a beta-oxidation pathway. In some embodiments, the beta-oxidation pathway is one or both of a mitochondrial beta-oxidation pathway and/or a peroxisomal beta-oxidation pathway. In other embodiments, the cellular metabolic pathway is an amino acid catabolic pathway. In some embodiments, the amino acid catabolic pathway is one or more branched chain amino acid catabolic pathway selected from the group consisting of a leucine, an isoleucine and/or a valine catabolic pathway which produces a branched acyl-CoA intermediate.

In some embodiments, the acyl-CoA comprises one or more of butyryl-CoA, valeryl-CoA, isovaleryl-CoA, trans-2-hexenoyl-CoA, 3-oxohexanoyl-CoA and/or hexanoyl-CoA.

In some embodiments, the at least one gene encodes a peroxisomal acyl-CoA oxidase, a peroxisomal acyl-CoA dehydrogenase, a mitochondrial acyl-CoA dehydrogenase, a mitochondrial isovaleryl-CoA dehydrogenase, a mitochondrial branched-chain α-keto acid dehydrogenase subunit, an isovaleryl-CoA dehydrogenase, a branched-chain aminotransferase, a glutaryl-CoA dehydrogenase, a keto-acid decarboxylase, a multifunctional beta-oxidation enzyme hydratase-dehydrogenase-epimerase, a peroxisomal oxoacyl thiolase, fatty-acid CoA ligase, a peroxisomal adenine nucleotide transporter, a cytosolic carnitine acetyltransferase, or a peroxisome biogenesis factor.

In some embodiments, the at least one gene is selected from one or more of the group consisting of POX1, POX2, POX3, POX4, POX5, POX6, BAT1, BAT2, YAT1, FAA1, ANT1, POT1, ACD1, IVD1, MFE1, PEX10, ARO10, GCDH1, GCDH2, ACD2, BCKD-E1α, BCKD-E1β, BCKD-E2, and homologues or orthologues thereof. In some embodiments, the at least one gene is selected from one or more of the group consisting of YALI0_D15708g, YALI0_B10406g, YALI0_D01265g, YALI0_F19910g, YALI0_D08690g, YALI0_F05038g, YALI0_D23815g, YALI0_D20768g, YALI0_D06930g, YALI0_E32835g, YALI0_F10857g, YALI0_D24750g, YALI0_E27654g, YALI0_C23859g, YALI0_E06567g, YALI0_C16797g, YALI0_F23749g, YALI0_B04906g, YALI0_D02387g, YALI0_E15378g, YALI0_E18568g, YALI0_D17864g, YALI0_E03058g, YALI0_F21197g, YALI0_E12573g, YALI0_F28567g, YALI0_F22121g, YALI0_D00671g, YALI0_D06215g, YALI0_D09383g, YALI0_D09493g, YALI0_A07733g and homologues or orthologues thereof.

In some embodiments, the engineered cell expresses two or more enzymes of the cannabinoid biosynthesis pathway selected from the group consisting of a cannabigerolic acid (CBGA) synthase, an unsaturated cannabigerolic acid (CBGA-uns) synthase, a ketolated cannabigerolic acid (CBGA-keto) synthase, a cannabigerol(iso)butyric acid (CBG(i)BA) synthase, a cannabigerovarinic acid (CBGVA) synthase, a tetrahydrocannabinoic acid (THCA) synthase, an unsaturated tetrahydrocannabinoic acid (THCA-uns) synthase, a ketolated tetrahydrocannabinoic acid (THCA-keto) synthase, a tetrahydrocannabi(iso)butolic acid (THC(i)BA) synthase, a tetrahydrocannabivarinic acid (THCVA) synthase, a cannabichromenic acid (CBCA) synthase, an unsaturated cannabichromenic acid (CBCA-uns) synthase, a ketolated cannabichromenic acid (CBCA-keto) synthase, a cannabichromene(iso)butyric acid (CBC(i)BA) synthase, a cannabichromvarinic acid (CBCVA) synthase, a cannabidiolic acid (CBDA) synthase, an unsaturated cannabidiolic acid (CBDA-uns) synthase, a ketolated cannabidiolic acid (CBDA-keto) synthase, a cannabi(iso)butolic acid (CBD(i)BA) synthase, a cannabidivarinic acid (CBDVA) synthase, a hexanoyl-CoA synthetase, a (iso)valeryl-CoA synthetase, a butyryl-CoA synthetase, a polyketide synthase, a polyketide cyclase, a geranyl pyrophosphate (GPP) synthase, a HMG-CoA synthase, a mevalonate kinase, an acetyl-CoA synthase, a acetyl-CoA carboxylase, a acetyl-CoA acetyltransferase/HMG-CoA reductase, a mevalonate-5-phosphate decarboxylase, an isopentenyl phosphokinase, and/or a hydroxymethylglutaryl-CoA reductase.

In some embodiments, the cell is capable of producing one or more cannabinoid derivatives selected from the group consisting of CBGA, CBGA-uns, CBGA-keto, CBGBA, CBGiBA, CBGVA, THCA, THCA-uns, THCA-keto, THCBA, THCiBA, THCVA, CBCA, CBCA-uns, CBCA-keto, CBCBA, CBCiBA, CBCVA, CBDA, CBDA-uns, CBDA-keto, CBDBA, CBDiBA, and CBDVA, and their decarboxylated derivatives, when cultured under suitable conditions. In some embodiments, the suitable conditions comprise supplementation with one or more of butyric acid, valeric acid, isovaleric acid, hexanoic acid, glucose, glycerol, hexanol, butanol, and/or oleic acid.

In some embodiments, the at least one gene comprises the combination of POX3, POX5 and one or both of ACD1 and IVD1, and wherein the cell demonstrates synergistic production of cannabinoid compared to a production of cannabinoid achieved with a cell comprising a deletion, inactivation, or mutation of only ACD1, IVD1 or the combination of POX3/POX5. In some embodiments, the at least one gene comprises the combination of YALI0_D15708g and YALI0_E12573g.

In some embodiments, the at least one gene comprises the combination of POX3, POX5, ACD1 and one or more of POX2, POX6, GCDH2, GCDH1, MFE1, YAT1, FAA1, ANT1, POT1, ACD2, and PEX10, and wherein the cell demonstrates an enhanced production of cannabinoid compared to a production of cannabinoid achieved with a cell comprising a deletion, inactivation or mutation of only the combination of POX3, POX5 and ACD1. In some embodiments, the at least one gene comprises the combination of IVD1 and at least one of BAT1 or BAT2, and wherein the cell demonstrates an enhanced production of one or more C5 branched-chain cannabinoid compared to a production of the same one or more C5 branched-chain cannabinoid achieved with a cell comprising a deletion, inactivation, or mutation of only IVD1. In some preferred embodiments, the one or more C5 branched-chain cannabinoid comprises one or more of CBGiBA, CBDiBA, CBCiBA, and/or THCiBA.

In some embodiments, the at least one gene comprises the combination of at least two of POX3, POX5 and ACD1. In some embodiments, the at least one gene comprises the combination of POX3, POX5, ACD1 and FAA1, and wherein the cell demonstrates a significantly improved production of C4 cannabinoids compared to a production of C4 cannabinoid achieved with a cell comprising a deletion, inactivation, or mutation of only POX3, POX5, and ACD1. In other embodiments, the at least one gene comprises the combination of POX3, POX5, ACD1 and YAT1, and wherein the cell demonstrates a significantly improved production of cannabinoids compared to a production of cannabinoid achieved with a cell comprising a deletion, inactivation, or mutation of only POX3, POX5, and ACD1.

In some embodiments, the at least one gene comprises the combination of IVD1 and at least one of BCKD-E1α and BCKD-E2, and wherein the cell demonstrates a reduced production of C5 branched cannabinoid product, compared to a production of C5 branched cannabinoids achieved with a cell comprising a deletion, inactivation or mutation of only IVD1. In some embodiments, the cell additionally demonstrates a significant increased production of CBGVA compared to a production of CBGVA achieved with a cell which comprises a deletion, inactivation or mutation of only IVD1. In some embodiments, the at least one gene further comprises at least one of POX3, POX5 and ACD1. In some embodiments, the at least one gene further comprises FAA1.

In some embodiments, the at least one gene comprises FAA1, ACD1, IVD1, POX3, POX5, and one or both of BCKD-BCKD-E1α and BCKD-E2. In some embodiments, the cell demonstrates a significantly improved production of C4 cannabinoids compared to a production of cannabinoid achieved with a cell comprising a deletion, inactivation, or mutation of only POX3, POX5, ACD1, IVD1 and BCKD-E1α.

In some embodiments, the at least one gene comprises FAA1. In some embodiments, the at least one gene further comprises ACD1 or IVD1.

In some embodiments, the cell is a yeast cell or a bacterial cell. In some embodiments, the yeast cell is a Yarrowia strain or a Saccharomyces strain.

Other aspects of the disclosure are directed to a method of producing a cannabinoid or derivative thereof comprising culturing a cell as disclosed herein under suitable conditions to produce the cannabinoid or derivative thereof. In some embodiments, the cannabinoid or derivative thereof comprises one or more of CBGA, CBGA-uns, CBGA-keto, CBGBA, CBGiBA, CBGVA, THCA, THCA-uns, THCA-keto, THCBA, THCiBA, THCVA, CBCA, CBCA-uns, CBCA-keto, CBCBA, CBCiBA, CBCVA, CBDA, CBDA-uns, CBDA-keto, CBDBA, CBDiBA, and CBDVA, or decarboxylated derivatives thereof. In some embodiments, the suitable condition comprises supplementing a culture media in which the cell is cultured with at least one of butyric acid, butyl-butyrate, valeric acid, isovaleric acid, hexanoic acid, hexanol, hexyl-hexanoate, butanol, oleic acid, glycerol or glucose. In some embodiments, the method comprises a step of isolating the cannabinoid or derivative thereof from the culture.

Another aspect of the invention is directed to a cell engineered to produce CBGiBA, CBGA-uns, CBGA-keto, THCiBA, THCA-uns, THCA-keto, CBCiBA. CBCA-uns, CBCA-keto, CBDiBA, CBD-uns, and/or CBD-keto, or decarboxylated derivatives thereof, wherein the cell comprises an inactivated or otherwise mutated IVD1, POT1, and/or MFE1 gene, and wherein the cell further expresses sufficient enzymes in the cannabinoid synthesis pathway to produce CBGiBA, CBGA-uns, CBGA-keto, THCiBA, THCA-uns, THCA-keto, CBCiBA. CBCA-uns, CBCA-keto, CBDiBA, CBD-uns, and/or CBD-keto, or decarboxylated derivatives thereof, when cultured under suitable conditions. In some embodiments, the suitable conditions comprise supplementation of a culture media in which the cell is cultured with butyric acid, butyl-butyrate, valeric acid, isovaleric acid, butanol, hexanol, hexanoic acid, hexyl-hexanoate, oleic acid, glycerol or glucose.

Other aspects of the disclosure are directed to a method of producing CBGiBA, CBGA-uns, CBGA-keto, THCiBA, THCA-uns, THCA-keto, CBCiBA, CBCA-uns, CBCA-keto, CBDiBA, CBD-uns, and/or CBD-keto, or decarboxylated derivatives thereof, by culturing a cell as disclosed herein under conditions suitable to produce the CBGiBA, CBGA-uns, CBGA-keto, THCiBA, THCA-uns, THCA-keto, CBCiBA. CBCA-uns, CBCA-keto, CBDiBA, CBD-uns, and/or CBD-keto, or decarboxylated derivatives thereof. In some embodiments, the suitable conditions comprise supplementing a culture media in which the cell is cultured with at least one of butyric acid, butyl-butyrate, valeric acid, isovaleric acid, butanol, hexanol, hexanoic acid, hexyl-hexanoate, oleic acid, glycerol or glucose.

In some embodiments, the method comprises a step of isolating CBGiBA, CBGA-uns, CBGA-keto, THCiBA, THCA-uns, THCA-keto, CBCiBA. CBCA-uns, CBCA-keto, CBDiBA, CBD-uns, and/or CBD-keto, or decarboxylated derivatives thereof from the culture.

Some other aspects of the present disclosure are directed to a compound according to either of the following structures:

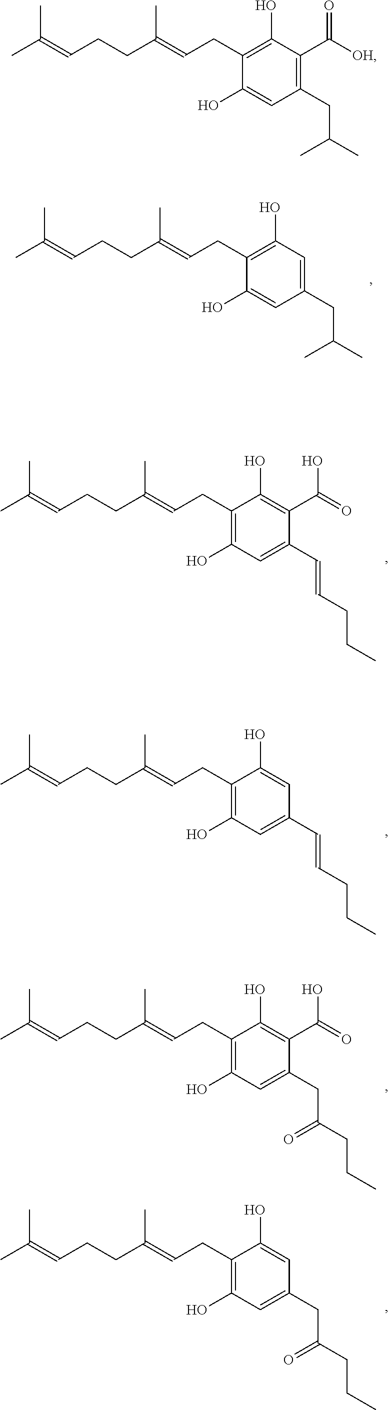

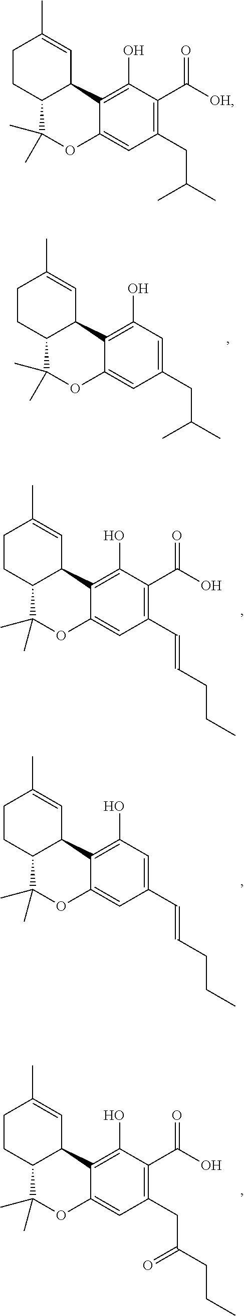

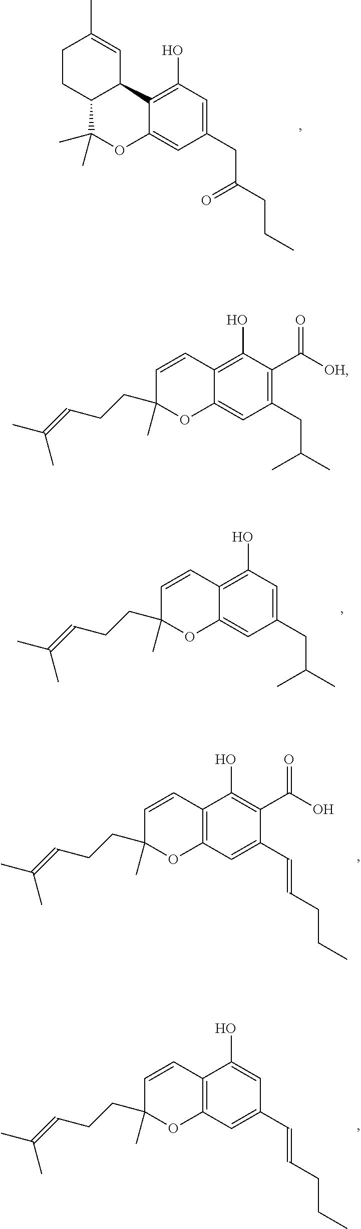

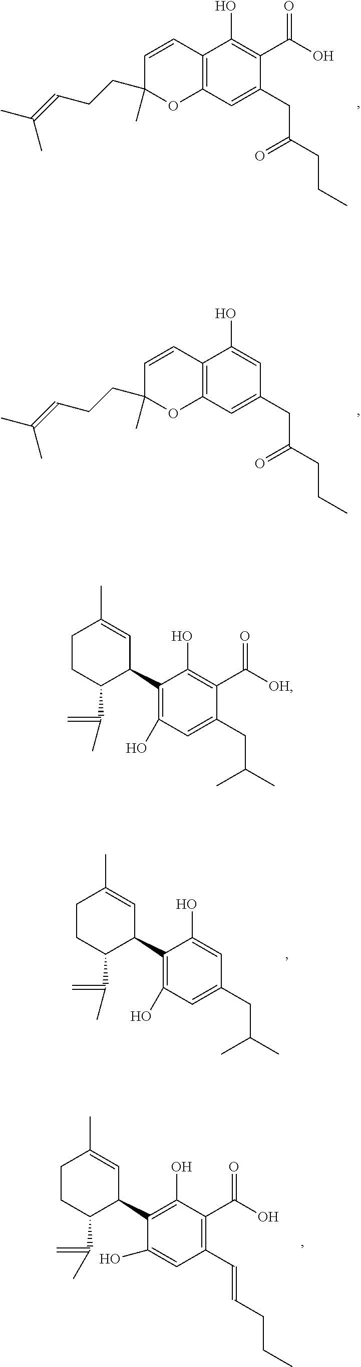

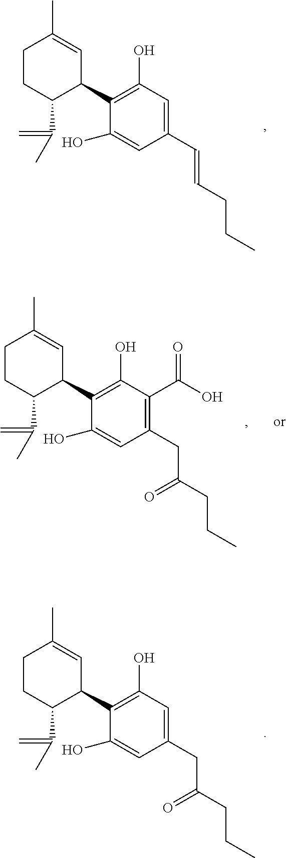

These and additional embodiments, along with their characteristics, advantages, and uses, will be further recognized from the description, claims, and drawings which follow.

### BRIEF DESCRIPTION OF THE DRAWINGS

FIG. 1 depicts the biosynthesis pathway for CBGA and major cannabinoids that are derived therefrom, including THC(V)A, CBD(V)A, and CBC(V)A.

FIG. 2 depicts the fatty acid β-oxidation pathway.

FIG. 3 depicts the leucine catabolism pathway.

FIGS. 4A-4B depict the expression of ACD1 and IVD1 in butyric acid feed fermentation.

FIGS. 5A-5B depict the expression of ACD1 and IVD1 in hexanoic acid feed fermentation.

FIG. 6 depicts the metabolic pathway of monounsaturated cannabinoid species.

FIG. 7 depicts the metabolic pathway of ketolated cannabinoid species.

### DETAILED DESCRIPTION OF THE INVENTION

### Some Definitions

“Identity” or “homology” refers to the extent to which the sequence of two or more nucleic acids or polypeptides is the same. In some embodiments, percent identity or homology between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity or homology, fractions are to be rounded to the nearest whole number. Percent identity or homology can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL ncbi.nlm.nih.gov for these programs. In a specific embodiment, percent identity or homology is calculated using BLAST2 with default parameters as provided by the NCBI.

The term “homologue” is intended to mean a nucleic acid sequence which possesses close sequence identity to the nucleic acid sequence of a recited gene and wherein both nucleic acid sequences are determined to be derived from the same ancestral gene, such as through speciation, either through phylogenetic analysis or through statistical analysis of the alignment between the sequences. When making the determination that two nucleic acids sequences are homologues through statistical analysis of the alignment between the sequences, tools which are widely known and available online, such as BLAST, may be utilized to make this determination. For purposes of this definition, alignments in BLAST given an expected value (E-value) of lower than 1×10-2, will be considered sufficient for determining that both nucleic acids derived from the same ancestral gene. The term “homologue” may also similarly be used to identify two amino acid sequences which possess close sequence homology and/or function and which are similarly determined to be encoded by and derived from the same ancestral gene. An “orthologue” is defined similarly as “homologue”, with the difference being the nucleic acid sequence which possesses close sequence identity to the nucleic acid sequence of a recited gene are both determined to be derived from the same ancestral gene through speciation.

The term “exogenous” is intended to mean that the referenced molecule or the referenced activity is introduced into the cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. When used in reference to a biosynthetic activity, the term refers to an activity that is introduced into the host. The source can be, for example, a homologous or heterologous encoding nucleic acid that expresses the referenced activity following introduction into the cell. Therefore, the term “endogenous” refers to a referenced molecule or activity that is present in the cell. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

The terms “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 5% as compared to a reference level, for example a decrease by at least 10%, a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance”, “improve” or “activate” are all used herein to generally mean an increase by a statically significant amount; for the avoidance of any doubt, the terms “increased”, “increase”, “enhance” or “activate” means an increase of at least 5% as compared to a reference level, an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. The phrases “increased production”, “enhanced production”, and “improved production” refer to an increase, enhancement, or improvement in an amount, yield, titer, concentration, quantity, volume, output, or flux of a particular measured product, including cannabinoids and derivatives thereof, compared to a reference level achieved by another method or in a control cell.

The term “statistically significant” or “significantly” refers to statistical significance and generally means a two-standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “acyl-CoA” generally refers to coenzyme A bound to a RC(O) radical, wherein R refers to an aliphatic chain, which can be saturated or unsaturated, and may optionally be substituted with a carbonyl (e.g. are ketolated). The term is defined to include at least butyryl-CoA, valeryl-CoA, isovaleryl-CoA, trans-2-hexenoyl-CoA, 3-oxohexanoyl-CoA and hexanoyl-CoA.

The term “C4 cannabinoid(s)” is defined as cannabinoid derived from a 4 carbon starter acyl compound such as butyric acid. Resulting C4 cannabinoids include CBGVA, THCVA, CBDVA, and CBCVA, and decarboxylated derivatives thereof.

The term “C5 cannabinoid(s)” is defined as cannabinoids derived from a 5 carbon starter acyl compound such as valeric acid or isovaleric acid. Resulting cannabinoids include CBGBA, THCBA, CBDBA, CBCBA, CBGiBA, THCiBA, CBDiBA, CBCiBA, and decarboxylated derivatives thereof.

The term “iC5 cannabinoid(s)” is defined as a subset of C5 cannabinoids derived from a branched chain 5 carbon starter acyl compound such as isovaleric acid. Resulting cannabinoids include CBGiBA, THCiBA, CBDiBA, CBCiBA, and decarboxylated derivatives thereof.

The term “C6 cannabinoid(s)” is defined as cannabinoids derived from a 6 carbon starter acyl compound such as hexanoic acid. Resulting cannabinoids include CBGA, THCA, CBDA, CBCA, CBGA-uns, THCA-uns, CBDA-uns, CBCA-uns, CBGA-keto, THCA-keto, CBDA-keto, CBCA-keto, and decarboxylated derivatives thereof.

The term “at least one gene” is defined as an open ended range which requires a minimum of one gene, but permits a combination of two or more genes, three or more genes, four or more genes, five or more genes, six or more genes, seven or more genes, eight or more genes, and so forth. To further identify and define particular genes recited in this application, the following index, which cites the Abbreviated Gene name, the corresponding Yarrowia lipolytica gene identification number, Accession number and description, is provided below.

| | | Accession | |
|---|---|---|---|
| Gene | YALI# | number | |
| ACD1 | YALI0_D15708g | Q6C8Y9 | mitochondrial acyl-CoA |
| | | | dehydrogenase |
| ACD2 | YALI0_B04906g | Q6CFQ1 | acyl-CoA dehydrogenase 2 |
| ANT1 | YALI0_E03058g | Q6C780 | peroxisomal adenine |
| | | | nucleotide transporter 1 |
| ARO10 | YALI0_D06930g | Q6CA04 | keto-acid decarboxylase |
| BAT1 | YALI0_D01265g | Q6CAN4 | branched-chain |
| | | | aminotransferase 1 |
| BAT2 | YALI0_F19910g | Q6C120 | branched-chain |
| | | | aminotransferase 2 |
| BCKD- | YALI0_D08690g | Q6C9S7 | the branched-chain α-keto- |
| E1α | | | acid dehydrogenase, |
| | | | subunit 1 alpha |
| BCKD- | YALI0_F05038g | Q6C2U7 | the branched-chain α-keto- |
| E1β | | | acid dehydrogenase, |
| | | | subunit 1 beta |
| BCKD-E2 | YALI0_D23815g | Q6C806 | the branched-chain α-keto- |
| | | | acid dehydrogenase, |
| | | | subunit 2 |
| BCKD-E3 | YALI0_D20768g | Q6C8C6 | the branched-chain α-keto- |
| | | | acid dehydrogenase, |
| | | | subunit 3 |
| ECH1 | YALI0_B10406g | Q6CF43 | enoyl-CoA hydratase |
| FAA1 | YALI0_D17864g | Q6C8Q3 | long-chain fatty-acid CoA |
| | | | ligase |
| GCDH1 | YALI0_C16797g | Q6CBP3 | glutaryl-coA dehydrogenase 1 |
| GCDH2 | YALI0_F23749g | Q6C0L1 | glutaryl-coA dehydrogenase 2 |
| IVD1 | YALI0_E12573g | Q6C643 | isovaleryl-CoA |
| | | | dehydrogenase |
| MFE1 | YALI0_E15378g | F2Z615 | multifunctional beta-oxidation |
| | | | enzyme hydratase- |
| | | | dehydrogenase-epimerase |
| PEX10 | YALI0_D02387g | Q6CAI8 | peroxisomal biogenesis |
| | | | factor 10 |
| POT1 | YALI0_E18568g | Q05493 | peroxisomal oxoacyl |
| | | | thiolase |
| POX1 | YALI0_E32835g | O74934 | peroxisomal acyl-CoA |
| | | | oxidase 1 |
| POX2 | YALI0_F10857g | O74935 | peroxisomal acyl-CoA |
| | | | oxidase 2 |
| POX3 | YALI0_D24750g | O74936 | peroxisomal acyl-CoA |
| | | | oxidase 3 |
| POX4 | YALI0_E27654g | F2Z627 | peroxisomal acyl-CoA |
| | | | oxidase 4 |
| POX5 | YALI0_C23859g | F2Z630 | peroxisomal acyl-CoA |
| | | | oxidase 5 |
| POX6 | YALI0_E06567g | Q6C6T0 | peroxisomal acyl-CoA |
| | | | oxidase 6 |
| YAT1 | YALI0_F21197g | Q6C0W4 | cytosolic carnitine |
| | | | acetyltransferase |

Engineered Cells Providing Increased Production of Cannabinoids and their Derivatives

Some aspects of the present disclosure are directed to a cell engineered to provide an increased production of cannabinoids and/or derivatives thereof compared to a control cell, the engineered cell expressing one or more enzymes of the cannabinoid biosynthesis pathway, wherein at least one gene encoding one or more enzymes or other proteins influencing metabolism of at least one acyl-CoA molecule of the cell has been deleted, inactivated, or otherwise mutated, thereby permitting the redirection of acyl-CoA molecules from a cellular metabolic pathway to a cannabinoid biosynthesis pathway to achieve the increased production of cannabinoids and/or derivatives thereof compared to a control cell, and wherein the control cell is lacking the same deletion, inactivation or other mutation to the at least one gene as the cell engineered to produce an increased production of cannabinoids and/or derivatives thereof.

In some embodiments, at least one gene is deleted which encodes one or more enzymes or other proteins influencing metabolism of at least one acyl-CoA molecule through a cellular metabolic pathway. The deletion may include total deletion of the at least one gene or partial deletion of the at least one gene. In some embodiments, the deletion includes deletion of a single allele, two alleles or all of the alleles of the same gene in the cell. Additionally, the deletion may also include deletion of a one or more alleles of a gene which is homologous to the at least one gene which is deleted in the cell. In some embodiments, more than one gene is deleted. In some embodiments, at least two, at least three, at least four, at least five, at least six, at least seven, at least eight, at least nine, at least ten, at least eleven or at least twelve or more genes are deleted. In some embodiments, deletion of the at least one gene does not result in death of the cell.

Techniques for deletion of a gene are not limited and include all techniques known to those of skill in the art for accomplishing partial or full deletion of a gene. In some embodiments, the deletion may be carried out by a one-step process, which results in a selectable phenotype, such as the methods described in U.S. Pat. Nos. 4,963,487 A and 4,713,337 A, both of which are hereby incorporated herein by reference in their entirety. In some embodiments, the deletion may be carried out by other protocols, including those described in U.S. Pat. Nos. 6,989,265 B2, 8,026,098 B2, and 9,249,428 B2, which are also hereby incorporated by reference in their entirety.

In some embodiments, at least one gene is inactivated or otherwise mutated which encodes one or more enzymes or other proteins influencing metabolism of at least one acyl-CoA molecule in a cellular metabolic pathway. In some embodiments, the inactivation or mutation provides knock-out of the gene, wherein the inactivated or mutated gene encodes a non-active protein or is no longer able to be transcribed or translated. In some embodiments, the inactivation or mutation provides knock-down of the gene, wherein the protein encoded by the inactivated or mutated gene has an activity which is reduced compared to a protein encoded by a non-inactivated or non-mutated gene. Such reduction in activity may constitute a reduction of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 83%, at least 85%, at least 88%, at least 90%, at least 92% at least 94%, at least 96% or more. In some embodiments, inactivation and/or mutation of the at least one gene will not result in death of the cell. In other embodiments, the reduction in activity of the protein encoded by the inactivated or mutated gene will not result in death of the cell.

In some embodiments, the inactivation or mutation includes inactivation or mutation of a single allele, two alleles or all the alleles of the same gene in the cell. Additionally, the inactivation or mutation may also include inactivation or mutation of one or more alleles of at least one gene which is homologous to the at least one gene which is inactivated or mutated in the cell. In some embodiments, more than one gene is inactivated or mutated. In some embodiments, at least two, at least three, at least four, at least five, at least six, at least seven, at least eight, at least nine, at least ten, at least eleven or at least twelve or more genes are inactivated or mutated. Techniques used to inactivate or mutate at least one gene are not limited and include those techniques known to those of skill in the art. In some embodiments, the inactivation and/or mutation may be accomplished through methods including, but not limited to recombination, such as through use of one or more gene disruption cassettes, such as described in U.S. Pat. No. 7,449,179 B2 and U.S. Pat. No. 6,534,315 B1, through repressing gene transcription as described in U.S. Pat. No. 6,576,469 B1, or through providing selective nucleic DNA insertions or deletions, such as through use of nucleases, including zinc finger nucleases (ZFN), I-CreI meganucleases, transcription activator-like effector nucleases (TALEN), and through use of a Clustered Regularly Interspaced Short Palindromic Repeats (CRISPR)-CRISPR associated (Cas) system, such as described in U.S. Pat. No. 7,951,925 B2, U.S. Pat. No. 8,106,255 B2, U.S. Pat. No. 7,897,372 B2, US 2011/0225664 A1, and US 2014/0170753 A1, which are all hereby incorporated by reference in their entirety.

In some embodiments, the enzyme or protein encoded by the at least one gene influences catabolism of acyl-CoA and/or influences one or both of a mitochondrial beta-oxidation pathway and a peroxisomal beta-oxidation pathway. For purposes of this invention, influencing catabolism of acyl-CoA and/or a mitochondrial or peroxisomal beta-oxidation pathway is defined as the ability of an enzyme or other protein to directly or indirectly modulate, regulate, promote, control, bias or otherwise effect the catabolism of an acyl-CoA molecule, 2-trans-enoyl CoA molecule, L-3-hydroxy acyl CoA molecule, or a 3-ketoacyl CoA molecule of a cell.

In some embodiments, the at least one gene may encode an enzyme which directly influences the mitochondrial or peroxisomal beta-oxidation pathway of a cell or directly influences the catabolism of an acyl-CoA molecule. In such embodiments, the at least one gene may encode a peroxisomal acyl-CoA oxidase, a mitochondrial acyl-CoA oxidase, a peroxisomal acyl-CoA dehydrogenase, a mitochondrial acyl-CoA dehydrogenase, a mitochondrial enoyl CoA hydratase, a peroxisomal enoyl CoA hydratase, a mitochondrial 3-hydroxy acyl CoA dehydrogenase, a peroxisomal 3-hydroxy acyl CoA dehydrogenase, a mitochondrial 3-ketoacyl CoA thiolase, a peroxisomal 3-ketoacyl CoA thiolase, or a multifunctional beta-oxidation enzyme hydratase-dehydrogenase-epimerase.

In some embodiments, the at least one gene may encode an enzyme or protein which indirectly influences the mitochondrial or peroxisomal beta-oxidation pathway of a cell and/or the catabolism of an acyl-CoA molecule. In such embodiments, the at least one gene may encode a peroxisomal oxoacyl thiolase, fatty-acid CoA ligase, a peroxisomal adenine nucleotide transporter, a cytosolic carnitine acetyltransferase, or a peroxisome biogenesis factor.

In some embodiments, the enzyme or protein encoded by the at least one gene influences a branched chain amino acid catabolic pathway. In some embodiments, the branched chain amino acid catabolic pathway is a leucine, isoleucine or valine catabolic pathway. In such embodiments, the at least one gene may encode a mitochondrial isovaleryl-CoA dehydrogenase, a mitochondrial branched-chain α-keto acid dehydrogenase subunit, an isovaleryl-CoA dehydrogenase, a branched-chain aminotransferase, or keto-acid decarboxylase.

In some embodiments, the cell is able to redirect acyl-CoA molecules from a cellular metabolic pathway to a cannabinoid biosynthesis pathway to achieve increased production of cannabinoids and/or derivative thereof compared to a control cell. This redirection of acyl-CoA molecules to a cannabinoid biosynthesis pathway results in an at least 5% increased level of one or more cannabinoids or derivatives thereof compared to the control cell. In other embodiments, the cell has at least a 10%, 15%, 20%, 25%, 30%, 35%, 40%, 45%, 50%, 55%, 60%, 65%, 70%, 75%, 80%, 85%, 90%, 95%, or 100% or more increase of one or more of cannabinoids and/or derivatives thereof compared to the control cell. In some embodiments, the cell has a 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5 fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 300-fold, 400-fold or more increase of one or more of cannabinoids and/or derivatives thereof compared to the control cell.

In some embodiments, the at least one gene encodes a peroxisomal acyl-CoA oxidase, a peroxisomal acyl-CoA dehydrogenase, a mitochondrial acyl-CoA dehydrogenase, a mitochondrial isovaleryl-CoA dehydrogenase, a mitochondrial branched-chain α-keto acid dehydrogenase subunit, an isovaleryl-CoA dehydrogenase, a branched-chain aminotransferase, a glutaryl-CoA dehydrogenase, a keto-acid decarboxylase, a multifunctional beta-oxidation enzyme hydratase-dehydrogenase-epimerase, a peroxisomal oxoacyl thiolase, fatty-acid CoA ligase, a peroxisomal adenine nucleotide transporter, a cytosolic carnitine acetyltransferase, or a peroxisome biogenesis factor.

In some embodiments, the at least one gene is selected from one or more of the group consisting of POX1, POX2, POX3, POX4, POX5, POX6, BAT1, BAT2, YAT1, FAA1, ANT1, POT1, ACD1, IVD1, MFE1, PEX10, ARO10, GCDH1, GCDH2, ACD2, BCKD-E1α, BCKD-E1β, BCKD-E2, and homologues or orthologues thereof. In some embodiments, the at least one gene is selected from one or more of the group consisting of YALI0_D15708g, YALI0_B10406g, YALI0_D01265g, YALI0_F19910g, YALI0_D08690g, YALI0_F05038g, YALI0_D23815g, YALI0_D20768g, YALI0_D06930g, YALI0_E32835g, YALI0_F10857g, YALI0_D24750g, YALI0_E27654g, YALI0_C23859g, YALI0_E06567g, YALI0_C16797g, YALI0_F23749g, YALI0_B04906g, YALI0_D02387g, YALI0_E15378g, YALI0_E18568g, YALI0_D17864g, YALI0_E03058g, YALI0_F21197g, YALI0_E12573g, YALI0_F28567g, YALI0_F22121g, YALI0_D00671g, YALI0_D06215g, YALI0_D09383g, YALI0_D09493g, YALI0_A07733g and homologues or orthologues thereof.

In some embodiments, the cell comprises exogenous polynucleotides which express a plurality of enzymes of the cannabinoid synthesis pathway including enzymes of the mevalonic acid, olivetolic acid (OA)/hexanoic acid, 2,4-dihydroxy-(iso)butylbenzoic acid (OA-(i)B)/(iso)valeric acid and divarinic acid (DVA)/butyric acid pathways. These enzymes of the cannabinoid synthesis pathway expressed in the cell may include acyl-CoA synthetases, such as hexanoyl-CoA, (iso)valeryl-CoA, and butyryl-CoA synthetases, polyketide synthases, polyketide cyclases, geranyl pyrophosphate (GPP) synthase, prenyltransferases, such as a cannabigerolic acid (CBGA) synthase, an unsaturated cannabigerolic acid (CBGA-uns) synthase, a ketolated cannabigerolic acid (CBGA-keto) synthase, a cannabigerol(iso)butyric acid (CBG(i)BA) synthase, a cannabigerovarinic acid (CBGVA) synthase, a tetrahydrocannabinoic acid (THCA) synthase, an unsaturated tetrahydrocannabinoic acid (THCA-uns) synthase, a ketolated tetrahydrocannabinoic acid (THCA-keto) synthase, a tetrahydrocannabi(iso)butolic acid (THC(i)BA) synthase, a tetrahydrocannabivarinic acid (THCVA) synthase, a cannabichromenic acid (CBCA) synthase, an unsaturated cannabichromenic acid (CBCA-uns) synthase, a ketolated cannabichromenic acid (CBCA-keto) synthase, a cannabichromene(iso)butyric acid (CBC(i)BA) synthase, a cannabichromvarinic acid (CBCVA) synthase, a cannabidiolic acid (CBDA) synthase, an unsaturated cannabidiolic acid (CBDA-uns) synthase, a ketolated cannabidiolic acid (CBDA-keto) synthase, a cannabi(iso)butolic acid (CBD(i)BA) synthase, and/or a cannabidivarinic acid (CBDVA) synthase, as well as HMG-CoA synthase, mevalonate kinase, acetyl-CoA synthase, acetyl-CoA carboxylase, acetyl-CoA acetyltransferase/HMG-CoA reductase, mevalonate-5-phosphate decarboxylase, isopentenyl phosphokinase, and hydroxymethylglutaryl-CoA reductase.

An expression vector or vectors can be constructed to include the exogenous nucleotide sequences coding for these enzymes and other polypeptides described herein, wherein the exogenous nucleotide sequences are operably linked to expression control sequences functional in the cell. Expression vectors applicable include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired enzymes or other products, and it is further understood that expression levels can be optimized to obtain sufficient expression using methods well known in the art and as disclosed herein.

In some embodiments, the cell expresses an acyl-CoA synthetase which is capable of producing an acyl-CoA from carboxylic acids with two to twenty-two carbons. In some embodiments, the acyl-CoA synthetase may produce butyryl-CoA, (iso)valeryl-CoA, hexanoyl-CoA, octanoyl-CoA, decanoyl-CoA, dodecanoyl-CoA, myristoyl-CoA, palmitoleyl-CoA, linoleyl-CoA, palmityl-CoA, and oleyl-CoA from (iso)valeric acid, hexanoic acid, octanoic acid, decanoic acid, dodecanoic acid, myristic acid, palmitic acid, linoleic acid, palmitic acid and oleic acid. The acyl-CoA synthase may be a wild-type enzyme or may be engineered to produce an acyl-CoA at a higher rate than wild-type acyl-CoA from Cannabis sativa. In some embodiments the cell expresses a fusion protein which comprises a protein having acyl-CoA synthetase activity fused to a protein having polyketide cyclase and/or a protein having polyketide synthase activity.

In some embodiments, the cell expresses a polyketide synthase which is capable of producing a tetraketide from one or more acyl-CoA substrates selected from carboxylic acids with two to twenty-two carbons, such as for example Acetyl-CoA, Butyryl-CoA, (iso)Valeryl-CoA, Hexanoyl-CoA, Trans-2-hexenoyl-CoA, 3-oxohexanoyl-CoA, Octanoyl-CoA, Decanoyl-CoA, Dodecanoyl-CoA, Myristoyl-CoA Palmitoleyl-CoA, Linoleyl-CoA, Palmityl-CoA, and Oleyl-CoA. The polyketide synthase may be a wild-type enzyme or may be engineered to produce tetraketide from the acyl-CoA substrate at a higher rate than wild-type polyketide synthase from Cannabis sativa. In some embodiments the cell expresses a fusion protein which comprises a protein having polyketide synthase activity fused to a protein having polyketide cyclase and/or protein having hexanoyl-CoA synthetase activity.

In some embodiments, the cell expresses a polyketide cyclase which is capable of cyclizing the polyketide synthase-produced tetraketide to the corresponding 6-alkyl-2,4-dihydroxy benzoic acid. In some embodiments cyclizing of the tetraketide produces olivetolic acid (OA), an OA analog, 2,3-dyhydroxy-6-(iso)butylbenzoic acid (OA-(i)B), an OA-(i)B analog, divarinic acid (DVA), or a DVA analog from a tetraketide, 2,4-dihydroxy-6-[(1E)-penten-1-yl]benzoic acid (OA-uns), or an OA-uns analog from a tetraketide, 2,4-dihydroxy-6-(2-oxopentyl)benzoic acid (OA-keto), or an OA-uns analog from a tetraketide. In some embodiments, the polyketide cyclase is capable of producing OA, an OA analog, OA-(i)B, an OA-(i)B analog, DVA, or a DVA analog, OA-uns, or an OA-uns analog, OA-keto, or an OA-keto analog at a higher rate than the wild-type polyketide cyclase from Cannabis sativa. In some embodiments, the cell expresses a fusion protein which comprises a protein having polyketide cyclase activity fused to a protein having polyketide synthase activity, a protein having acyl-CoA synthetase activity, a protein having prenyltransferase activity and/or a protein having CBGA, CBG(i)BA, CBGVA, CBGA-uns or CBGA-keto synthase activity.

In some embodiments, the cell expresses an aromatic soluble prenyltransferase or a membrane-bound prenyltransferase which is capable of transferring a prenyl group, such as a geranyl group to pyrophosphate to produce geranyl pyrophosphate (GPP). In some embodiments, the prenyltransferase demonstrates a preference for transferring a geranyl group over a farnesyl group to a pyrophosphate containing group. In some embodiments, the prenyltransferase is capable of producing GPP at a higher rate than a wild-type prenyltransferase from Cannabis sativa. In some embodiments, the cell expresses a fusion protein which comprises a protein having prenyltransferase activity fused to a protein having polyketide synthase activity, a protein having acyl-CoA synthetase activity, a protein having polyketide cyclase activity and/or a protein having CBGA, CBG(i)BA, CBGVA, CBGA-uns or CBGA-keto synthase activity.

In some embodiments, the cell expresses a wild-type or engineered cannabigerolic acid (CBGA), unsaturated cannabigerolic acid (CBGA-uns), ketolated cannabigerolic acid (CBGA), cannabigerol(iso)butyric acid (CBG(i)BA), and/or cannabigerovarinic acid (CBGVA) synthase which is capable of forming CBGA, CBGA-uns, CBGA-keto, CBG(i)BA or CBGVA from GPP and at least one of OA, OA-(i)B or DVA. In some embodiments, the CBGA, CBGA-uns, CBGA-keto, CBG(i)BA, and/or CBGVA synthase is capable of producing CBGA and/or CBGVA at a higher rate than a wild-type CBGA or CBGBA synthase from Cannabis sativa.

In some embodiments, the cell expresses a wild-type or engineered cannabichromenic acid (CBCA), unsaturated cannabichromenic acid (CBCA-uns), ketolated cannabichromenic acid (CBCA-keto), cannabichromene(iso)butyric acid (CBC(i)BA), and cannabichromvarinic acid (CBCVA) synthase which is capable of forming CBCA, CBCA-uns, CBCA-keto, CBC(i)BA or CBCVA from CBGA, CBGA-uns, CBGA-keto, CBG(i)BA or CBCVA. In some embodiments, the CBCA or CBCVA synthase is capable of producing CBCA and/or CBCVA at a higher rate than a wild-type CBCA or CBCBA synthase from Cannabis sativa.

In some embodiments, the cell expresses a wild-type or engineered tetrahydrocannabinoic acid (THCA), unsaturated tetrahydrocannabinoic acid (THCA-uns), ketolated tetrahydrocannabinoic acid (THCA-keto), tetrahydrocannabi(iso)butolic acid (THC(i)BA), and tetrahydrocannabivarinic acid (THCVA) synthase which is capable of forming THCA, THCA-uns, THCA-keto, THC(i)BA or THCVA from CBGA, CBGA-uns, CBGA-keto, CBG(i)BA or CBGVA. In some embodiments, the THCA or THCVA synthase is capable of producing THCA or THCVA at a higher rate than a wild-type THCA or THCVA synthase from Cannabis sativa.

In some embodiments, the cell expresses a wild-type or engineered cannabidiolic acid (CBDA), unsaturated cannabidiolic acid (CBDA-uns), ketolated cannabidiolic acid (CBDA-keto), cannabi(iso)butolic acid (CBD(i)BA), and/or cannabidivarinic acid (CBDVA) synthase which is capable of forming CBDA, CBDA-uns, CBDA-keto, CBD(i)BA or CBDVA from CBGA, CBGA-uns, CBGA-keto, CBG(i)BA or CBGVA. In some embodiments, the CBDA or CBDVA synthase is capable of producing CBDA or CBDVA at a higher rate than a wild-type CBDA or CBDVA synthase from Cannabis sativa.

In some embodiments, the engineered cell expresses two or more enzymes of the cannabinoid biosynthesis pathway selected from the group consisting of a cannabigerolic acid (CBGA) synthase, an unsaturated cannabigerolic acid (CBGA-uns) synthase, a ketolated cannabigerolic acid (CBGA-keto) synthase, a cannabigerol(iso)butyric acid (CBG(i)BA) synthase, a cannabigerovarinic acid (CBGVA) synthase, a tetrahydrocannabinoic acid (THCA) synthase, an unsaturated tetrahydrocannabinoic acid (THCA-uns) synthase, a ketolated tetrahydrocannabinoic acid (THCA-keto) synthase, a tetrahydrocannabi(iso)butolic acid (THC(i)BA) synthase, a tetrahydrocannabivarinic acid (THCVA) synthase, a cannabichromenic acid (CBCA) synthase, an unsaturated cannabichromenic acid (CBCA-uns) synthase, a ketolated cannabichromenic acid (CBCA-keto) synthase, a cannabichromene(iso)butyric acid (CBC(i)BA) synthase, a cannabichromvarinic acid (CBCVA) synthase, a cannabidiolic acid (CBDA) synthase, an unsaturated cannabidiolic acid (CBDA-uns) synthase, a ketolated cannabidiolic acid (CBDA-keto) synthase, a cannabi(iso)butolic acid (CBD(i)BA) synthase, a cannabidivarinic acid (CBDVA) synthase, a hexanoyl-CoA synthetase, a (iso)valeryl-CoA synthetase, a butyryl-CoA synthetase, a polyketide synthase, a polyketide cyclase, a geranyl pyrophosphate (GPP) synthase, a HMG-CoA synthase, a mevalonate kinase, an acetyl-CoA synthase, a acetyl-CoA carboxylase, a acetyl-CoA acetyltransferase/HMG-CoA reductase, a mevalonate-5-phosphate decarboxylase, an isopentenyl phosphokinase, and/or a hydroxymethylglutaryl-CoA reductase.

In some embodiments, the cell is capable of producing one or more cannabinoid derivatives selected from the group consisting of CBGA, CBGA-uns, CBGA-keto, CBGBA, CBGiBA, CBGVA, THCA, THCA-uns, THCA-keto, THCBA, THCiBA, THCVA, CBCA, CBCA-uns, CBCA-keto, CBCBA, CBCiBA, CBCVA, CBDA, CBDA-uns, CBDA-keto, CBDBA, CBDiBA, and CBDVA, and their decarboxylated derivatives when cultured under suitable conditions. In some embodiments, the suitable conditions include supplementation in the presence of a carbon source and/or nutrients. The carbon source or nutrients are not particularly limited and may include any carbon source or nutrient described herein or well known in the art. In some embodiments, the carbon source or nutrient comprises one or more of butyric acid, butanol, valeric acid, isovaleric acid, hexanoic acid, hexanol, glucose, glycerol, and/or oleic acid.

In some embodiments, the cell produces an increased production of cannabinoid derivatives compared to a control cell which has not been engineered to metabolize a reduced number of acyl-CoA molecules and/or a reduced number of amino acids by deletion, inactivation or other mutation of at least one gene which encodes an enzyme or other protein which influences the beta-oxidation pathway, influences the catabolism of acyl-CoA and/or the branched-chain amino acid catabolism pathway. In some embodiments, the cell has at least a 10%, 15%, 20%, 25%, 30%, 35%, 40%, 45%, 50%, 55%, 60%, 65%, 70%, 75%, 80%, 85%, 90%, 95%, or 100% or more increased production of cannabinoid derivatives compared to the control cell. In some embodiments, the cell has a 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5 fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 300-fold, 400-fold or more increased flux of cannabinoid derivatives compared to the control cell.

In some embodiments, the at least one gene comprises the combination of two or more, three or more, four or more, five or more, six or more, seven or more, eight or more, nine or more, ten or more, eleven or more, or twelve or more genes which directly or indirectly influence a beta-oxidation pathway, directly or indirectly influence acyl-CoA metabolism and/or directly or indirectly influence catabolism of a branched chain amino acid.

In some embodiments, the at least one gene comprises the combination of POX3, POX5 and one or both of ACD1 and IVD1, and wherein the cell demonstrates synergistic production of cannabinoid compared to a production of cannabinoid achieved with a cell comprising a deletion, inactivation or mutation of only ACD1, IVD1 or the combination of POX3/POX5. In some embodiments, the at least one gene comprises the combination of YALI0_D15708g and YALI0_E12573g.

In some embodiments, the at least one gene comprises the combination of POX3, POX5, ACD1 and one or more of POX2, POX6, GCDH1, GCDH2, MFE1, YAT1, FAA1, ANT1, POT1, ACD2, and PEX10, and wherein the cell demonstrates an enhanced production of cannabinoid compared to a production of cannabinoid achieved with a cell comprising a deletion, inactivation or mutation of only the combination of POX3, POX5 and ACD1. In some embodiments, the at least one gene comprises the combination of IVD1 and at least one of BAT1 or BAT2, and wherein the cell demonstrates an enhanced production of one or more C5 branched-chain cannabinoid compared to a production of the same one or more C5 branched-chain cannabinoid achieved with a cell comprising a deletion, inactivation, or mutation of only IVD1. In some preferred embodiments, the one or more C5 branched-chain cannabinoid comprises one or more of CBGiBA, CBDiBA, CBCiBA, and/or THCiBA.

In some embodiments, the at least one gene comprises the combination of at least two of POX3, POX5 and ACD1. In some embodiments, the at least one gene comprises the combination of POX3, POX5, ACD1 and FAA1, and wherein the cell demonstrates a significantly improved production of C4 cannabinoids compared to a production of C4 cannabinoid achieved with a cell comprising a deletion, inactivation, or mutation of only POX3, POX5, and ACD1. In other embodiments, the at least one gene comprises the combination of POX3, POX5, ACD1 and YAT1, and wherein the cell demonstrates a significantly improved production of cannabinoids compared to a production of cannabinoid achieved with a cell comprising a deletion, inactivation, or mutation of only POX3, POX5, and ACD1.

In some embodiments, the at least one gene comprises the combination of IVD1 and at least one of BCKD-E1α and BCKD-E2, and wherein the cell demonstrates a reduced production of C5 branched cannabinoid product, compared to a production of C5 branched cannabinoids achieved with a cell comprising a deletion, inactivation or mutation of only IVD1. In some embodiments, the cell additionally demonstrates a significant increased production of CBGVA compared to a production of CBGVA achieved with a cell which comprises a deletion, inactivation or mutation of only IVD1. In some embodiments, the at least one gene further comprises the combination of at least two of POX3, POX5, ACD1 and FAA1.

In some embodiments, the cell expresses a mutant farnesyl pyrophosphate synthase protein (FPPS). In some embodiments, the mutant FPPS is ERG20.A28 and is capable of producing an increased ratio of GPP to FPP. In some embodiments, the cell has an elevated level of GPP as compared to a control cell.

In some embodiments, the cell may be a microorganism or a plant cell. Suitable cells may include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha (now known as Pichia angusta), Kluyveromyces sp., Kluyveromyces lactis, Kluyveromyces marxianus, Schizosaccharomyces pompe, Dekkera bruxellensis, Arxula adeninivorans, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, Yarrowia lipolytica and the like. In some embodiments, the cell is a protease-deficient strain of Saccharomyces cerevisiae. In some embodiments, the cell is Yarrowia lipolytica. In some embodiments, the cell is a eukaryotic cell other than a plant cell. In some embodiments, the cell is a plant cell. In some embodiments, the cell is a plant cell that does not normally produce a cannabinoid, a cannabinoid derivative or analogue, a cannabinoid precursor, or a cannabinoid precursor derivative or analogue. In some embodiments, the cell disclosed herein is cultured in vitro.

In some embodiments, the cell is a prokaryotic cell. Suitable prokaryotic cells may include, but are not limited to, any of a variety of laboratory strains of Escherichia coli, Lactobacillus sp., Salmonella sp., Shigella sp., and the like. See, e.g., Carrier et al, (1992) J. Immunol. 148:1176-1181; U.S. Pat. No. 6,447,784; and Sizemore et al. (1995) Science 270:299-302. Examples of Salmonella strains which can be employed may include, but are not limited to, Salmonella typhi and S. typhimurium. Suitable Shigella strains may include, but are not limited to, Shigella flexneri, Shigella sonnei, and Shigella disenteriae. Typically, the laboratory strain is one that is non-pathogenic. Non-limiting examples of other suitable bacteria may include, but are not limited to, Bacillus subtilis, Pseudomonas putida, Pseudomonas aeruginosa, Pseudomonas mevalonii, Rhodobacter sphaeroides, Rhodobacter capsulatus, Rhodospirillum rubrum, Rhodococcus sp., and the like.

### Methods of Producing a Cannabinoid

Some aspects of the present disclosure are directed to a method of producing a cannabinoid or derivative thereof comprising culturing a cell as disclosed herein under suitable conditions to produce the cannabinoid or derivative thereof.

Depending on the cell, the appropriate culture medium may be used. For example, descriptions of various culture media may be found in “Manual of Methods for General Bacteriology” of the American Society for Bacteriology (Washington D.C., USA, 1981). As used here, “medium” as it relates to the growth source refers to the starting medium be it in a solid or liquid form. “Cultured medium”, on the other hand and as used here refers to medium (e.g. liquid medium) containing microbes that have been fermentatively grown and can include other cellular biomass. The medium generally includes one or more carbon sources, nitrogen sources, inorganic salts, vitamins and/or trace elements.

Exemplary carbon sources include sugar carbons such as sucrose, glucose, galactose, fructose, mannose, isomaltose, xylose, panose, maltose, arabinose, cellobiose and 3-, 4-, or 5-oligomers thereof. Other carbon sources include alcohol carbon sources such as methanol, ethanol, glycerol, butanol and hexanol. Other carbon sources include acid and esters such as acetate, formate, fatty acids having four to twenty-two carbon atoms or fatty acid esters thereof. Other carbon sources can include renewal feedstocks and biomass. Exemplary renewal feedstocks include cellulosic biomass, hemicellulosic biomass and lignin feedstocks. Mixed carbon sources can also be used, such as a fatty acid and a sugar as described herein. In some embodiments, the suitable condition comprising supplementing a culture media in which the cell is cultured comprises supplementing with at least one of butyric acid, butanol, butyl-butyrate, valeric acid, isovaleric acid, hexanoic acid, hexanol, hexyl-hexanoate, oleic acid, glycerol or glucose.

The culture conditions can include, for example, liquid culture procedures as well as fermentation and other large-scale culture procedures. Useful yields of the products can be obtained under aerobic culture conditions. An exemplary growth condition for achieving one or more cannabinoid products includes aerobic culture or fermentation conditions. In certain embodiments, the microbial organism can be sustained, cultured or fermented under aerobic conditions.

Substantially aerobic conditions include, for example, a culture, batch fermentation or continuous fermentation such that the dissolved oxygen concentration in the medium remains between 5% and 100% of saturation. The percent of dissolved oxygen can be maintained by, for example, sparging air, pure oxygen or a mixture of air and oxygen.

The culture conditions can be scaled up and grown continuously for manufacturing cannabinoid product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cannabinoid product. Generally, and as with non-continuous culture procedures, the continuous and/or near-continuous production of cannabinoid product will include culturing a cannabinoid producing organism on sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of cannabinoid product can be utilized in, for example, fed-batch fermentation and batch separation; fed-batch fermentation and continuous separation, or continuous fermentation and continuous separation. Examples of batch and continuous fermentation procedures are well known in the art.

In some embodiments, the cells are grown in stirred tank fermenters with feed supplementation (sugars with or without organic acids) where the dissolved oxygen, temperature, and pH are controlled according to the optimal growth and production process. In some embodiments, aqueous non-miscible organic solvents are supplemented to dissolve added organic acids or extract the cannabinoid products as they are being synthesized. In some embodiments, these solvents may include, but are not limited to, isopropyl myristate (IPM), diisobutyl adipate, decane, dodecane, hexadecane or another organic solvent with log P>5. The later number (log P) is defined as the log of a compound's partition between water and octanol and is a standard parameter of a compound's hydrophobicity (the larger the log P the less soluble in water). Depending on the fermentation process, the products can be isolated and purified using different methods.

If no organic cosolvent is used and the targeted cannabinoid(s) is being secreted to the culture supernatant, different methods can be applied. In one embodiment, an aqueous miscible organic solvent (ethanol, acetonitrile, etc.) is added to dissolve the products. In some embodiments, a simple filtration, ultrafiltration or centrifugation can remove the cells and the aqueous media evaporated to dryness or to a small volume from which the cannabinoid product will precipitate or crystalize. Alternatively, the cell supernatant can be extracted with an aqueous immiscible organic solvent (ethyl acetate, heptane, decane, etc.) to extract the cannabinoids. Evaporation of the organic solvent and a possible recrystallization will produce pure cannabinoid. If the cannabinoid products are not secreted to the media and are trapped inside the cell, different methods for their extraction and purification can be utilized. In some embodiments, cells are disrupted using mechanical methods or by suspension in appropriate lysis buffers from which the cannabinoids can be extracted with an organic aqueous immiscible solvent (ethyl acetate, hexane, decane, methylene chloride, etc.). In other embodiments, cells may be suspended in an organic solvent (ethanol, methanol, methylene chloride, etc.) that extracts the cannabinoids from the cells.

In some embodiments, an organic solvent is required during growth that is separated at the end of the fermentation. Back extraction with basic aqueous solvent or a different organic solvent with low boiling point and high polarity (ethanol, acetonitrile, etc.) will remove the cannabinoids. Isolation can then involve a simple pH shift if water is used, or an evaporation if organic solvents are used. In both cases, a recrystallization step may be required at the end to improve purity of the product.

In some embodiments, the methods further comprise a step of purifying or isolating the cannabinoids, derivatives or analogues thereof from the culture. Methods of isolation are not limited and may be any suitable method known in the art. Purification methods include, for example, extraction procedures as well as methods that include continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration.

In some embodiments, the cannabinoids, cannabinoid derivatives and cannabinoid analogues as recited herein produced by the method are not limited. In some embodiments, cannabinoids may include, but are not limited to, cannabichromene (CBC) type (e.g. cannabichromenic acid), cannabigerol (CBG) type (e.g. cannabigerolic acid), cannabidiol (CBD) type (e.g. cannabidiolic acid), Δ9-trans-tetrahydrocannabinol (Δ9-THC) type (e.g. Δ9-tetrahydrocannabinolic acid), Δ8-trans-tetrahydrocannabinol (Δ8-THC) type, cannabicyclol (CBL) type, cannabielsoin (CBE) type, cannabinol (CBN) type, cannabinodiol (CBND) type, and cannabitriol (CBT) type. In some embodiments, the cannabinoids, cannabinoid derivatives and cannabinoid analogues comprise one or more of cannabigerolic acid (CBGA), unsaturated cannabigerolic acid (CBGA-uns), ketolated cannabigerolic acid (CBGA-keto), cannabigerolic acid monomethylether (CBGAM), cannabigerol (CBG), unsaturated cannabigerol (CBG-uns), ketolated cannabigerol (CBG-keto), cannabigerol monomethylether (CBGM), cannabigerovarinic acid (CBGVA), unsaturated cannabigerovarinic acid (CBGVA-uns), ketolated cannabigerovarinic acid (CBGVA-keto), cannabigerovarin (CBGV), unsaturated cannabigerovarin (CBGV-uns), ketolated cannabigerovarin (CBGV-keto), cannabichromenic acid (CBCA), unsaturated cannabichromenic acid (CBCA-uns), ketolated cannabichromenic acid (CBCA-keto), cannabichromene (CBC), unsaturated cannabichromene (CBC-uns), ketolated cannabichromene (CBC-keto), cannabichromevarinic acid (CBCVA), unsaturated cannabichromevarinic acid (CBCVA-uns), ketolated cannabichromevarinic acid (CBCVA-keto), cannabichromevarin (CBCV), unsaturated cannabichromevarin (CBCV-uns), ketolated cannabichromevarin (CBCV-keto), cannabidiolic acid (CBDA), unsaturated cannabidiolic acid (CBDA-uns), ketolated cannabidiolic acid (CBDA-keto), cannabidiol (CBD), unsaturated cannabidiol (CBD-uns), ketolated cannabidiol (CBD-keto), cannabidiol monomethylether (CBDM), cannabidiol-C4 (CBD-C4), cannabidivarinic acid (CBDVA), unsaturated cannabidivarinic acid (CBDVA-uns), ketolated cannabidivarinic acid (CBDVA-keto), cannabidivarin (CBDV), cannabidiorcol (CBD-C1), Δ9-tetrahydrocannabinolic acid A (THCA-A), Δ9-tetrahydrocannabinolic acid B (THCA-B), unsaturated Δ9-tetrahydrocannabinolic acid A (THCA-A-uns), ketolated Δ9-tetrahydrocannabinolic acid A (THCA-A-keto), unsaturated Δ9-tetrahydrocannabinolic acid B (THCA-B-uns), ketolated Δ9-tetrahydrocannabinolic acid B (THCA-B-keto), Δ9-tetrahydrocannabinol (THC), unsaturated Δ9-tetrahydrocannabinol (THC-uns), ketolated Δ9-tetrahydrocannabinol (THC-keto), Δ9-tetrahydrocannabinolic acid-C4 (THCA-C4), Δ9-tetrahydrocannabinol-C4 (THC-C4), Δ9-tetrahydrocannabivarinic acid (THCVA), unsaturated Δ9-tetrahydrocannabivarinic acid (THCVA-uns), ketolated Δ9-tetrahydrocannabivarinic acid (THCVA-keto), Δ9-tetrahydrocannabivarin (THCV), Δ9-tetrahydrocannabiorcolic acid (THCA-C1), Δ9-tetrahydrocannabiorcol (THC-C1), A7-cis-iso-tetrahydrocannabivarin, Δ8-tetrahydrocannabinolic acid (Δ8-THCA), Δ8-tetrahydrocannabinol (Δ8-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C4, (CBN-C4), cannabivarin (CBV), cannabinol-C2 (CNB-C2), cannabiorcol (CBN-C1), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC). In a preferred embodiment, the cannabinoid or derivative thereof produced by the method comprises one or more of CBGA, CBGA-uns, CBGA-keto, CBGBA, CBGiBA, CBGVA, THCA, THCA-uns, THCA-keto, THCBA, THCiBA, THCVA, CBCA, CBCA-uns, CBCA-keto, CBCBA, CBCiBA, CBCVA, CBDA, CBDA-uns, CBDA-keto, CBDBA, CBDiBA, and CBDVA, and their decarboxylated derivatives.

In some embodiments, the production of one or more of these cannabinoids is increased 10%, 15%, 20%, 25%, 30%, 35%, 40%, 45%, 50%, 55%, 60%, 65%, 70%, 75%, 80%, 85%, 90%, 95%, or 100% or more compared to use of a method with a control cell, which does not possess deletion, inactivation or other mutation to at least one gene which influences a beta-oxidation pathway, influences catabolism of acyl-CoA molecules, and/or catabolism of a branched chain amino acid. In some embodiments, the production of at least one of these cannabinoids is increased 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5 fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 300-fold, 400-fold or more compared to use of a method with a control cell which does not possess deletion, inactivation or other mutation to at least one gene which influences a beta-oxidation pathway, influences catabolism of acyl-CoA molecules, and/or catabolism of a branched chain amino acid.

### Engineered Cells and Methods to Produce C5 Branched Cannabinoids, Unsaturated Cannabinoids, and Ketolated Cannabinoids

Some aspects of the present disclosure are also directed to a cell engineered to produce CBGiBA, CBGA-uns, CBGA-keto, THCiBA, THCA-uns, THCA-keto, CBCiBA. CBCA-uns, CBCA-keto, CBDiBA, CBD-uns, and/or CBD-keto, or decarboxylated derivatives thereof, wherein the cell comprises an inactivated or otherwise mutated IVD1, POT1, and/or MFE1 gene, and wherein the cell further expresses sufficient enzymes in the cannabinoid synthesis pathway to produce CBGiBA, CBGA-uns, CBGA-keto, THCiBA, THCA-uns, THCA-keto, CBCiBA. CBCA-uns, CBCA-keto, CBDiBA, CBD-uns, and/or CBD-keto, or decarboxylated derivatives thereof, when cultured under suitable conditions, such as those already described herein. In some embodiments, the suitable conditions comprise supplementation of a culture media in which the cell is cultured with butyric acid, valeric acid, isovaleric acid, hexanoic acid, oleic acid, butanol, hexanol, glycerol or glucose.

Some aspects of the present disclosure are directed to a method of producing CBGiBA, CBGA-uns, CBGA-keto, THCiBA, THCA-uns, THCA-keto, CBCiBA, CBCA-uns, CBCA-keto, CBDiBA, CBD-uns, and/or CBD-keto, or decarboxylated derivatives thereof, by culturing a cell as disclosed herein under conditions suitable to produce the CBGiBA, CBGA-uns, CBGA-keto, THCiBA, THCA-uns, THCA-keto, CBCiBA. CBCA-uns, CBCA-keto, CBDiBA, CBD-uns, and/or CBD-keto, or decarboxylated derivatives thereof. In some embodiments, the suitable conditions comprise supplementing a culture media in which the cell is cultured with at least one of butyric acid, valeric acid, isovaleric acid, hexanoic acid, hexanol, butanol, oleic acid, glycerol or glucose. In some embodiments, the method comprises a step of isolating CBGiBA, CBGA-uns, CBGA-keto, THCiBA, THCA-uns, THCA-keto, CBCiBA. CBCA-uns, CBCA-keto, CBDiBA, CBD-uns, and/or CBD-keto, or decarboxylated derivatives thereof from the culture, utilizing methods already described herein.

Some other aspects of the present disclosure are directed to a compound according to either of the following structures:

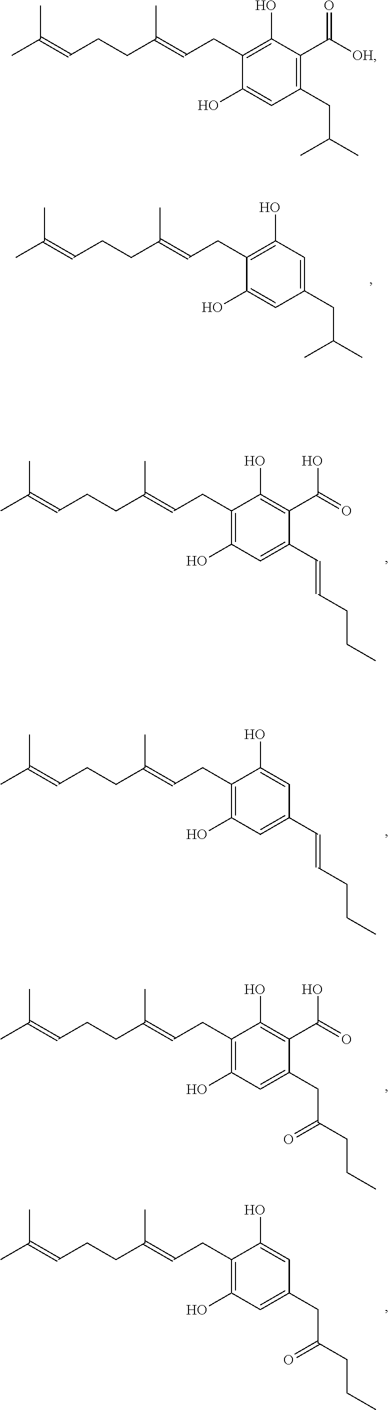

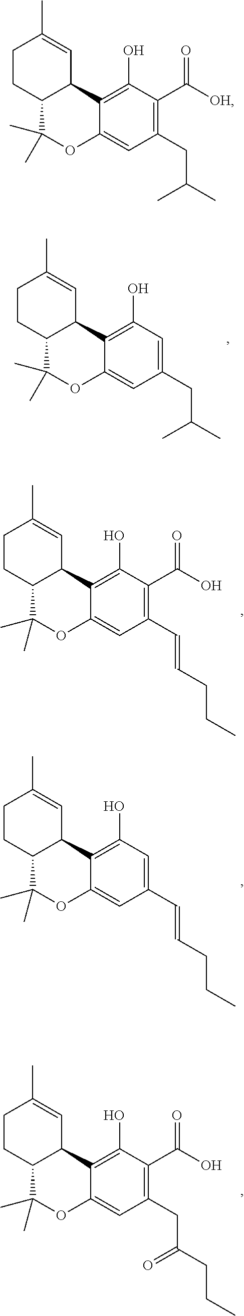

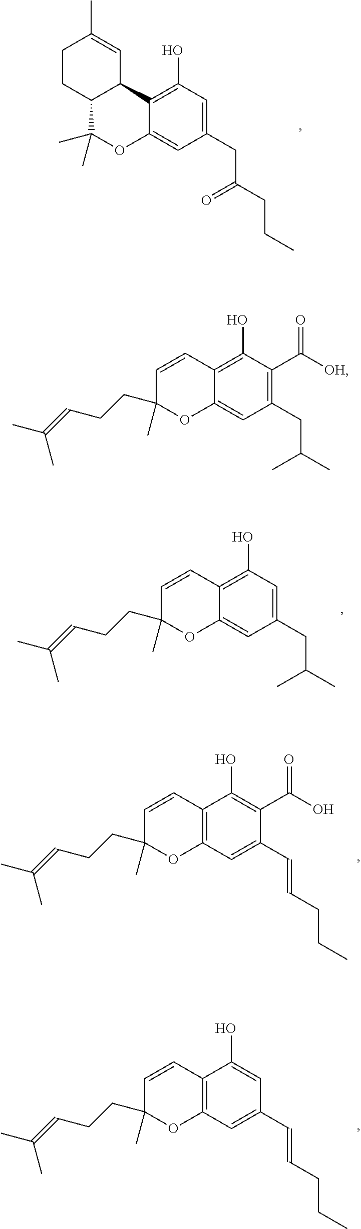

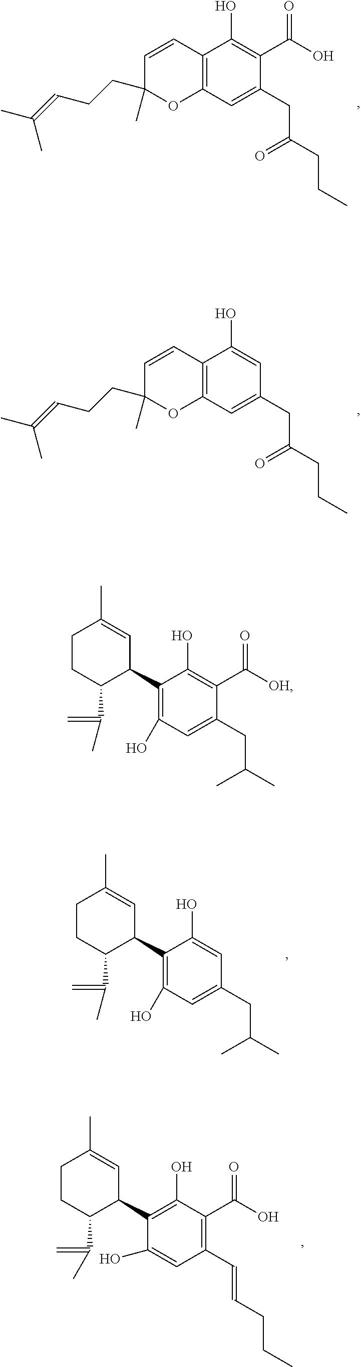

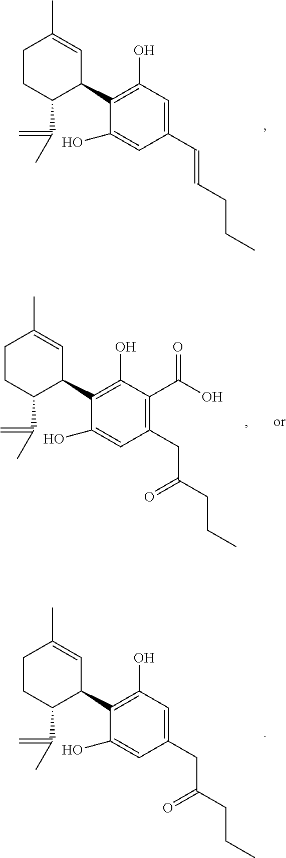

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, or cell, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

The invention and its advantages are further expounded by way of the foregoing non-limiting examples.

### Example 1. Rationale for Deleting ACD1 and ECH1 in Yarrowia lipolytica Cannabinoid Producing Strains

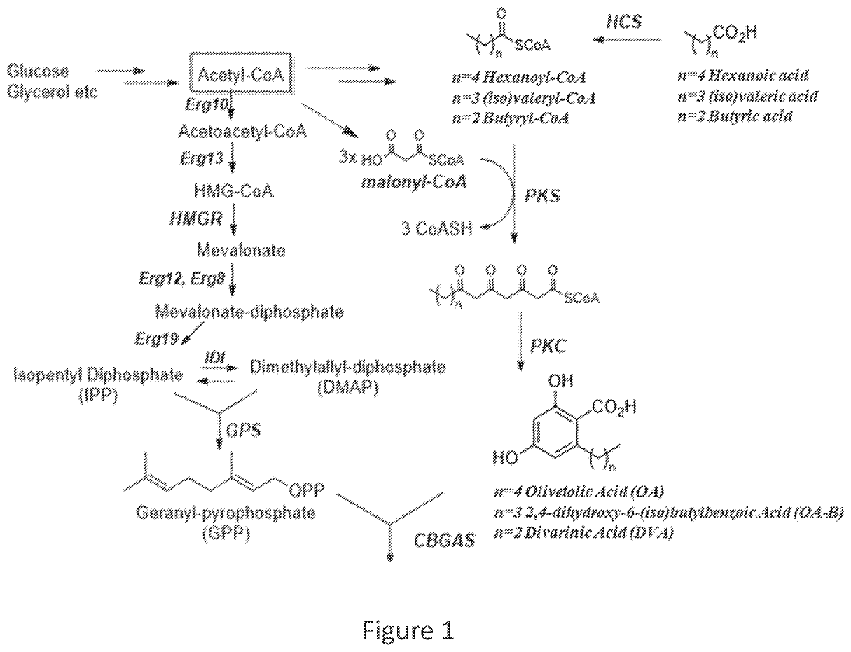

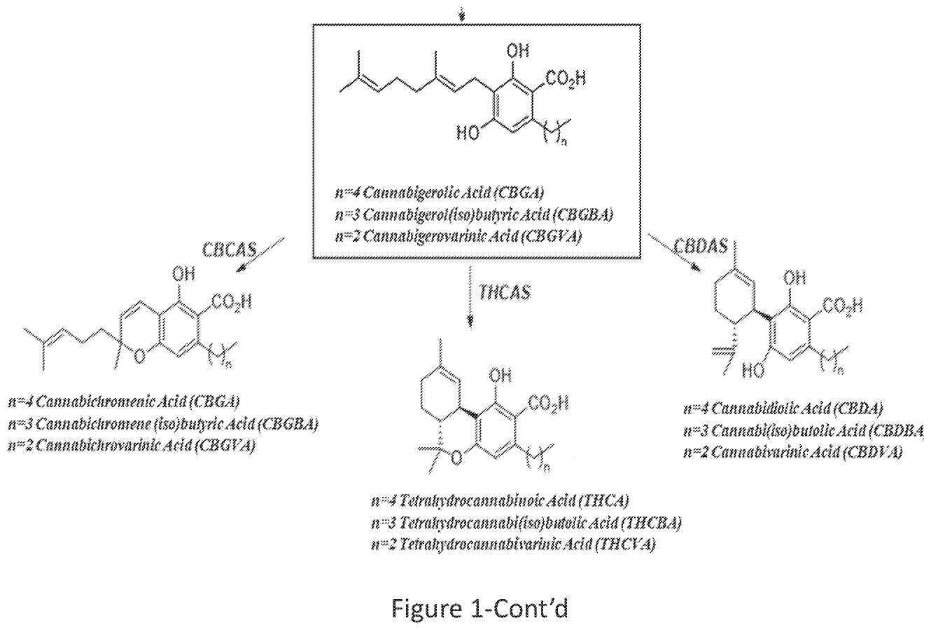

An aspect of this invention is metabolic engineering to conserve the cellular pool of acyl-CoA molecules for improved cannabinoid production. An overview of cannabinoid metabolic pathways including some of the important acyl-CoA molecules (hexanoyl-CoA, butyryl-CoA, (iso)valeryl-CoA, trans-2-hexenoyl-CoA and 3-oxohexanoyl-coA) is shown in FIG. 1 and Table 1. Hexanoyl-CoA, trans-2-hexenoyl-CoA, 3-oxohexanoyl-coA, (iso)valeryl-CoA and Butyryl-CoA are key precursors to the formation of the intermediates olivetolic acid (OA), OA-uns, OA-keto, OA-(i)B and divarinic acid (DVA), respectively, which are condensed with GPP to form the initial cannabinoids CBGA, CBGA-uns, CBGA-keto, CBG(i)BA and CBGVA, respectively. These initial cannabinoids (CBGA, CBGA-uns, CBGA-keto, CBG(i)BA and CBGVA) can be converted to various terminal cannabinoids shown in FIG. 1. To improve microbial cannabinoid production, we hypothesized that cells need to direct Hexanoyl-CoA, trans-2-hexenoyl-CoA, 3-oxohexanoyl-coA, (iso)valeryl-CoA or Butyryl-CoA towards OA, OA-(i)B or DVA and away from competing pathways.

TABLE 1
Nomenclatures of several cannabinoid intermediates and their starter CoA molecules.

| | | | THCAS |
|---|---|---|---|
| Starter CoA | PKS-PKC products | CBGAS products | products |
| Butyryl-CoA 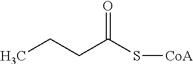 | VDAL, DVO, DVA | CBGVA | THCVA |
| Valeryl-CoA 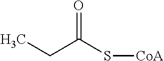 | PDAL-B, OL-B, OA-B | CBGBA | THCBA |
| Isovarleryl-CoA 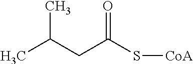 | PDAL-iB, OL-iB, OA-iB | CBGiBA | THCiBA |
| Hexanoyl-CoA 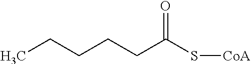 | PDAL, OL, OA | CBGA | THCA |
| Trans-2-hexenoyl-CoA 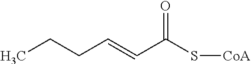 | PDAL-uns, OL-uns, OA-uns | CBGA-uns | THCA-uns |
| 3-Oxohexanoyl-CoA 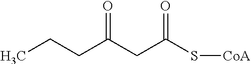 | PDAL-keto, OL-keto, OA-keto | CBGA-keto | THCA-keto |

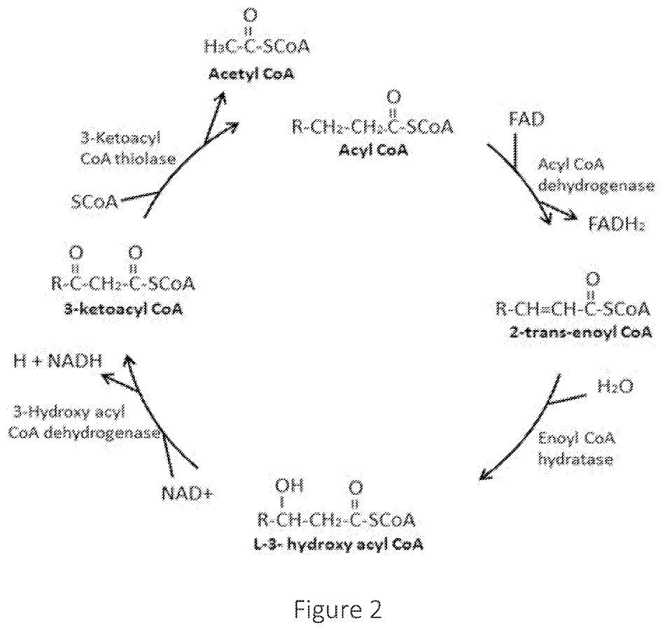

Fatty acid β-oxidation is the process of breaking down a long-chain acyl-CoA molecule to acetyl-CoA molecules, and it is the main pathway competing with the cannabinoid formation pathway for Hexanoyl-CoA, and Butyryl-CoA. This process involves a variety of enzymes, with the four key enzymes being, in order, acyl-CoA dehydrogenase, enoyl-CoA hydratase, hydroxyacyl-CoA dehydrogenase, and ketoacyl-CoA thiolase (FIG. 2).

In mammals, β-oxidation occurs in both mitochondria and peroxisomes, whereas plants and most fungi harbor the β-oxidation cycle only in the peroxisomes. In fact, the literature states that “Yeast and fungi, including the Ascomycetes Saccharomyces cerevisiae, Yarrowia lipolytica, and Candida tropicalis, appear to only possess peroxisomal β-oxidation capability, specifically the D-isomer pathway” (Kunau et al., 1988; Hiltunen et al., 1992; Kurihara et al., 1992; Smith et al., 2000).

Y. lipolytica contains six acyl-CoA oxidases, encoded by the POX1 to POX6 genes, which catalyze the limiting step (Acyl-CoA dehydrogenase) of peroxisomal β-oxidation. The POX genes have been characterized and the acyl-chain specificity for each encoded enzyme has been determined (Wang et al., 1999) making subsets of these genes targets for disruption to generate engineered cells that have increased acyl-CoA availability for making cannabinoids. In contrast, to our knowledge, there have been no reports verifying mitochondrial β-oxidation gene targets in Y. lipolytica. However, mitochondrial β-oxidation has been reported in the filamentus fungi Aspergillus nidulans and was shown to be important for the catabolism of short chain (C₄-C₆) fatty acids such as butyryl-CoA and hexanoyl-CoA (Maggio-Hall and Keller, 2004). Thus, we used the Human, mitochondrial short/branched chain specific acyl-CoA dehydrogenase (GenBank: AAH13756.1) in a BLAST search against the Y. lipolytica genome. The top hit in the BLAST search was YALI0_D15708g, with 58.7% sequence identity (amino acid) to the Human, mitochondrial short/branched chain specific acyl-CoA dehydrogenase. This gene is referred to as ACD1 (Acyl-CoA dehydrogenase). In addition, we used the Human, mitochondrial short/branched chain specific enoyl-CoA hydratase (GenBank: EAA57779) in a BLAST search against the Y. lipolytica genome. YALI0_B10406g was the top hit in the BLAST search and was 52.5% identical (amino acid) to the Human, mitochondrial short/branched chain specific enoyl-CoA hydratase, of which we refer to it as ECH1 (Enoyl-CoA hydratase). Even though the amino acid % identity was low for both the ACD and ECH homologs, we decided to disrupt these genes in Y. lipolytica strains that were engineered to produce cannabinoids to evaluate if inactivating these enzyme activities would increase the ability of the engineered cells to make cannabinoids, presumably through increased availability of Hexanoyl-CoA and Butyryl-CoA.

### Example 2. Assays Used to Evaluate Cannabinoid Formation from Glucose with or without Fatty Acid Supplementation

Individual clones from agar plates were used to inoculate, in triplicate, 0.5 mL YDCM medium (yeast nitrogen base+nitrogen, 6.71 g/L; Casamino acids, 10 g/L; Dextrose monohydrate, 66 g/L; MES hydrate 19.5, g/L; pH adjusted to 6.5 with KOH) in 96 deep well blocks which were grown at 30° C. with 990 rpm shaking. After 24 h, 2 μL from each preculture was used to inoculate 0.5 mL YDCM medium in 96 deep well blocks. At the time of inoculation, the cultures were supplemented with 2.5 mM of either butyric acid, butyl-butyrate, butanol, valeric acid, hexanoic acid, hexyl-hexanoate, hexanol, or no fatty acid and incubated at 30° C. with 990 rpm shaking. After 24 h of growth the cultures were supplemented with an additional 3 mM of the same fatty acid and incubated at 30° C. with 990 rpm shaking. After an additional 24 h (48 h total), cultures were quenched with Quench solution (0.5 mL ethanol with and 0.1 mg/mL pentyl-benzoic acid) and submitted for LC analysis.

### Example 3. Analytical Chemistry Methods of Cannabinoids

All samples after quenching with equal volume of Quench solution were centrifuged and analyzed by HPLC-MS
Column: 2.1×50 mm COSMOCORE PBr (Nacalai USA, Inc.)
Buffers:
Buffer A: Water, 0.1% formic acid
Buffer B: Acetonitrile, 0.1% formic acid
Flow rate: 0.45 mL/min
Column Temperature: 50 Celsius
Injection vol.: 1 μL

TABLE 2
Gradient Method

| Time (minutes) | Buffer B % |
|---|---|
| 0 | 20 |
| 2.3 | 70 |
| 4.2 | 80 |
| 4.3 | 100 |
| 5.3 | 100 |
| 5.4 | 20 |

TABLE 3
Retention time of cannabinoids disclosed in this application.

| Compound | Retention Time |
|---|---|
| VDAL | 1.18 |
| DVO | 1.71 |
| DVA | 1.99 |
| CGBVA | 3.67 |
| THCVA | 4.23 |
| PDAL-B | 1.79 |
| OL-B | 2.14 |
| OA-B | 2.53 |
| CBGBA | 3.84 |
| THCBA | 4.48 |
| PDAL-iB | 1.64 |
| OL-iB | 2.05 |
| OA-iB | 2.20 |
| CBGiBA | 3.77 |
| THCiBA | 4.38 |
| PDAL | 2.16 |
| OL | 2.43 |
| OA | 2.53 |
| CBGA | 4.03 |
| THCA | 4.79 |
| PDAL-uns | 1.88 |
| OL-uns | 2.41 |
| OA-uns | 2.67 |
| CBGA-uns | 4.07 |
| THCA-uns | 4.71 |

Retention times for all cannabinoid compounds listed in Table 1 are recorded in Table 3. All compounds were verified by LC-MS analysis using mass spectrometry of theoretical molecular weights.

### Example 4. ACD1 and ECH1 Deletion Strains SB-01213, SB-01209, SB-01215 and SB-01211

ACD1 and ECH1 were disrupted in two cannabinoid producing strains, SB-00889 and SB-01052. The genotypes of these strains are listed in Table 34. Both strains have been engineered to convert supplemental butyric acid or hexanoic acid to CBGVA or CBGA, respectively. One key difference between the two strains is that SB-0889 has a complete peroxisomal β-oxidation pathway whereas SB-01052 has had certain acyl-CoA oxidases (POX3 and POX5) inactivated. Standard molecular biology cloning techniques were used to disrupt ACD1 and ECH1 in strains SB-00889 and SB-01052. Deletion of ACD1 in strains SB-00889 and SB-01052 resulted in strains SB-01213 and SB-01209, respectively; and deletion of ECH1 in strains SB-00889 and SB-01052 resulted in strains SB-01215 and SB-01211, respectively.

### Example 5. Evaluating CBGA Formation from Glucose Plus Hexanoic Acid Feeds in ACD1 Deletion Strain

Clones from SB-01209 and SB-01213, along with the parental control strains were evaluated under the conditions described in Example 2. As shown in FIGS. 1 and 2, upon activation of hexanoic acid to hexanoyl-CoA, the latter is both a direct precursor to OA, which is an immediate precursor to the cannabinoid CBGA, and a substrate for β-oxidation. In the assay performed, all the hexanoic acid was consumed by the strains in question, thus the amount of OA and CBGA produced is a measure of how efficiently these strains are at converting hexanoyl-CoA to cannabinoids versus breaking down hexanoyl-CoA via β-oxidation. The amount of OA and CBGA produced by the strains is shown in Table 4. The data in Table 4 show that ACD1 inactivation in cannabinoid producing strains increased both OA and CBGA titers from hexanoic acid (compare SB-01213 to SB-00889). This shows that inactivation of mitochondrial β-oxidation can improve cannabinoid production. Comparing SB-01052 (Δpox3Δpox5) and SB-00889 shows that disruption of pox3 and pox5 results in improved cannabinoid production. Additionally, the SB-01209 clones have both mitochondrial (ACD1) and peroxisomal (POX3 & POX5) β-oxidation genes inactivated show that this combination has a synergistic effect on increasing cannabinoid production. The ECH1 inactivation strains were evaluated in a manner identical to the ACD1 inactivation strains, however none of the clones produced more OA and/or CBGA (data not shown).

TABLE 4
OA and CBGA produced by ACD1 deletion strains
from glucose and hexanoic acid supplementation

| | OA | | CBGA | | |
|---|---|---|---|---|---|
| Strain | μM | STDEV | μM | STDEV | OA + CBGA μM |
| SB-00889 | 0 | 0 | 741 | 41 | 741 |
| SB-01213_01 | 347 | 18 | 831 | 29 | 1178 |
| SB-01213_04 | 377 | 42 | 768 | 95 | 1145 |
| SB-01213_11 | 382 | 10 | 784 | 61 | 1166 |
| SB-01213_12 | 416 | 21 | 802 | 21 | 1218 |
| SB-01052 | 59 | 25 | 872 | 74 | 931 |
| SB-01209_01 | 445 | 48 | 823 | 136 | 1268 |
| SB-01209_04 | 441 | 14 | 836 | 12 | 1277 |
| SB-01209_05 | 469 | 21 | 824 | 42 | 1293 |
| SB-01209_11 | 547 | 42 | 770 | 29 | 1317 |

### Example 6. Inactivation of IVD1 in Yarrowia lipolytica Cannabinoid Producing Strains

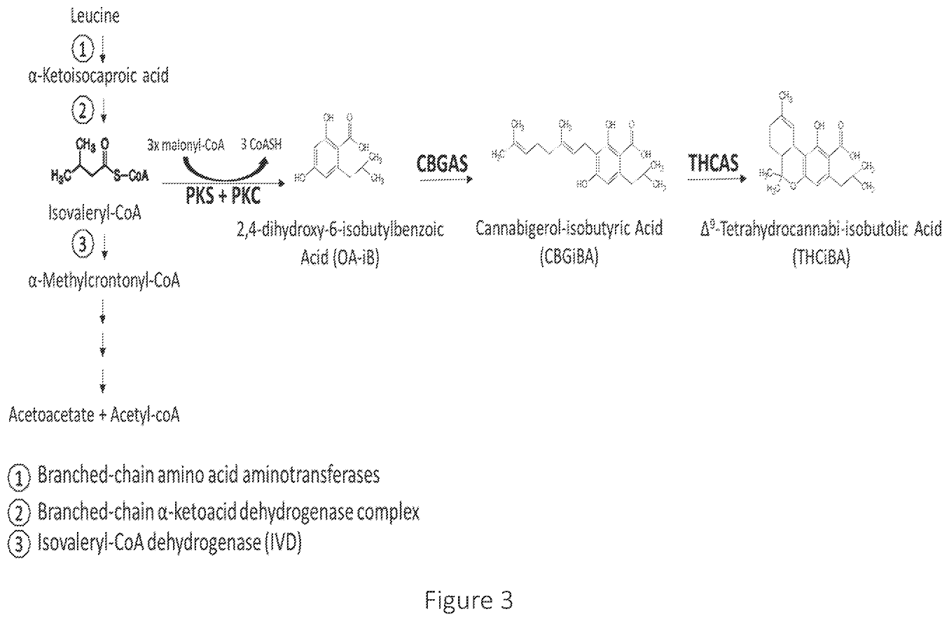

Based on the success of the ACD1 deletion strains, additional mitochondrial acyl-CoA dehydrogenases were targeted for inactivation to further conserve short-chain and medium chain acyl-CoA molecules to increase cannabinoid production. Specifically, twelve ACD and ECH homologs (Table 5) were identified then disrupted in cannabinoid producing strains using standard molecular biology techniques. One targeted gene, once disrupted, resulting in a strain that was able to produce rare cannabinoids. This gene is an ACD homolog that appears to encode an isovaleryl-CoA dehydrogenase (IVD1; YALI0_E12573g) is likely a part of the leucine catabolic pathway (FIG. 3). Leucine is deaminated by an aminotransferase to α-ketoisocaproic acid, which is subsequently decarboxylated by mitochondrial branched-chain α-keto acid dehydrogenase complex (BCKD complex) to isovaleryl-coA, which subsequently is dehydrated by Isovaleryl-CoA dehydrogenase, and eventually is decarboxylated to acetyl-CoA and acetoacetic acid. Inactivation of IVD1 should lead to accumulation of isovaleryl-CoA which is a potential substrate to produce rare cannabinoids.

TABLE 5
ACD and ECH homologs identified in the genome of Y. lipolytica.

| | Genes |
|---|---|
| ECH homologs | YALI0_F28567g |
| | YALI0_F22121g |
| | YALI0_D00671g |
| | YALI0_D06215g |
| | YALI0_D09383g |
| | YALI0_D09493g |
| | YALI0_A07733g |
| ACD homologs | YALI0_E12573g |
| | YALI0_E06567g |
| | YALI0_F23749g |
| | YALI0_C16797g |
| | YALI0_B04906g |

Standard molecular biology cloning techniques were used to disrupt IVD1 in SB-01052 (POX3 and POX5 inactivated) and SB-01268 (POX3, POX5 and ACD1 inactivated) that have been engineered to produce CBGVA and CBGA from supplemental butyric acid and hexanoic acid, respectively, resulting in strains SB-01343 and SB-01345. The genotypes of these strains are listed in Table 34.

### Example 7. Evaluating Cannabinoid Formation in IVD1 Deletion Strains without Fatty Acid Supplementation

Strains with IVD1 inactivated were assayed for their ability to produce cannabinoids without fatty acid supplementation as described in Example 2. The results are shown in Table 6. These data show that SB-01343 and SB-01355 which have IVD1 inactivated produce the C5-branched chain cannabinoids OA-iB and CBGiBA, whereas the parental strains did not. Both parental strains have POX3 and POX5 inactivated, but in addition, SB-01268 has ACD1 inactivated. Based on the genotypes of the strains and the data generated it appears that IVD1 inactivation is required for making branched chain cannabinoids whereas inactivation of ACD1, POX3, and POX5 is insufficient.

TABLE 6
C5-branced chain OA and C5-branced chain
CBGA produced by IVD1 deletion strains

| | OA-iB | | CBGiBA | | OA-iB + CBGiBA |
|---|---|---|---|---|---|
| Strain | μM | STDEV | μM | STDEV | μM |
| SB-01052 | 0 | 0 | 0 | 0 | 0 |
| SB-01343-13 | 816 | 7 | 41 | 1 | 857 |
| SB-01268 | 0 | 0 | 0 | 0 | 0 |
| SB-01355-06 | 666 | 4 | 6 | 10 | 672 |

### Example 8: Evaluating Cannabinoids Formation in IVD1 Strains with Fatty Acid Supplementation

To evaluate if IVD1 and ACD1 disruptions have synergistic outcomes on final cannabinoid titers, SB-01343 and SB-01355, along with the parental control strains, SB-01052 and SB-01268, were assayed under the fermentation conditions described in Example 2. The results were recorded in Table 7, 8 and 9. In the assays performed, all the supplemented butyric acid, valeric acid, and hexanoic acid were consumed by the strains in question, thus the amount of DVA+CBGVA, OA-B+CBGBA, OA+CBGA produced is a measure of how efficiently these strains convert butyryl-CoA, valeryl-CoA and hexanoyl-CoA to cannabinoids versus breaking down these molecules via various catabolic processes.

SB-01052 can be used as the baseline as the POX3 and POX5 inactivation is common to all strains. When ACD1 (SB-01268) or IVD1 (SB-01343) is inactivated in SB-01052 the strains make less total cannabinoid when supplemented with butyric acid or valeric acid. However, when both ACD1 and IVD1 (SB-01355) are inactivated in SB-01052 the strains make more total cannabinoid when supplemented with butyric acid or valeric acid. These data suggest that ACD1 and IVD1 have redundant activity on butyryl-CoA and valeryl-CoA and that inactivation of both are required to improve the production of cannabinoids derived from butyric acid or valeric acid.

When ACD1 (SB-01268) or IVD1 (SB-01343) is inactivated in SB-01052 the strains make more total cannabinoid when supplemented with hexanoic acid. These data suggest that deletion of either ACD1 or IVD1 can improve the production of cannabinoids derived from hexanoic acid. However, When ACD1 and IVD1 (SB-01355) are inactivated in SB-01052 the strains do not make more total cannabinoids than the individual deletions when supplemented with hexanoic acid.

TABLE 7
Cannabinoid profiles produced by ACD1 and IVD1
deletion strains supplemented with butyric acid

| | DVA | | CBGVA | | DVA + CBGVA |
|---|---|---|---|---|---|
| Strain | μM | STDEV | μM | STDEV | μM |
| SB-01052 | 607 | 6 | 104 | 4 | 711 |
| SB-01343-13 | 104 | 2 | 0 | 0 | 106 |
| SB-01268 | 439 | 8 | 46 | 2 | 485 |
| SB-01355-06 | 1398 | 14 | 67 | 3 | 1465 |

TABLE 8
Cannabinoid profiles produced by ACD1 and IVD1 deletion
strains supplemented with valeric acid.

| | OAB | | CBGBA | | OAB + CBGBA |
|---|---|---|---|---|---|
| Strain | μM | STDEV | μM | STDEV | μM |
| SB-01052 | 370 | 24 | 203 | 6 | 573 |
| SB-01343-13 | 232 | 19 | 127 | 2 | 359 |
| SB-01268 | 418 | 6 | 115 | 3 | 533 |
| SB-01355-06 | 575 | 78 | 101 | 10 | 677 |

TABLE 9
Cannabinoid profiles produced by ACD1 and IVD1 deletion
strains supplemented with hexanoic acid.

| | OA | | CBGA | | OA + CBGA |
|---|---|---|---|---|---|
| Strain | μM | STDEV | μM | STDEV | μM |
| SB-01052 | 554 | 107 | 174 | 39 | 728 |
| SB-01343-13 | 747 | 11 | 227 | 2 | 974 |
| SB-01268 | 802 | 10 | 187 | 1 | 989 |
| SB-01355-06 | 754 | 14 | 178 | 2 | 932 |

### Example 9: Experimental Procedures Used to Evaluate Expression of IVD1 and ACD1 Genes

Individual clones from agar plates were used to inoculate, in triplicate, 2 mL YDCM medium in 24 deep well blocks which were grown at 30° C. with 250 rpm shaking. After 24 h, 10 μL from each preculture was used to inoculate 2 mL YDCM medium in 24 deep well blocks which were incubated at 30° C. with 250 rpm shaking. After 24 h, cultures were supplemented with 3 mM of either butyric acid or hexanoic acid. After an additional 6 h (30 h total), cells from 1 mL cultures were harvested by centrifugation at 6000g for 3 min. Supernatants were removed and cell-pellets were frozen and kept at −80° C. freezer. RNA was extracted from individual cell pellets using the commercially available Quick-RNA Fungal/Bacterial Miniprep Kit (Zymo Research). RNA samples were reverse transcribed to cDNA using the LunaScript® RT SuperMix Kit (NEB), which were subsequently used for qPCR analysis. The qPCR primer sequences used for ACD1, IVD1 and the reference gene TAF10 are Sequence ID 1 to 6.

### Example 10: Evaluating the Expression of ACD1 and IVD1 Genes in Cannabinoid Producing Strains

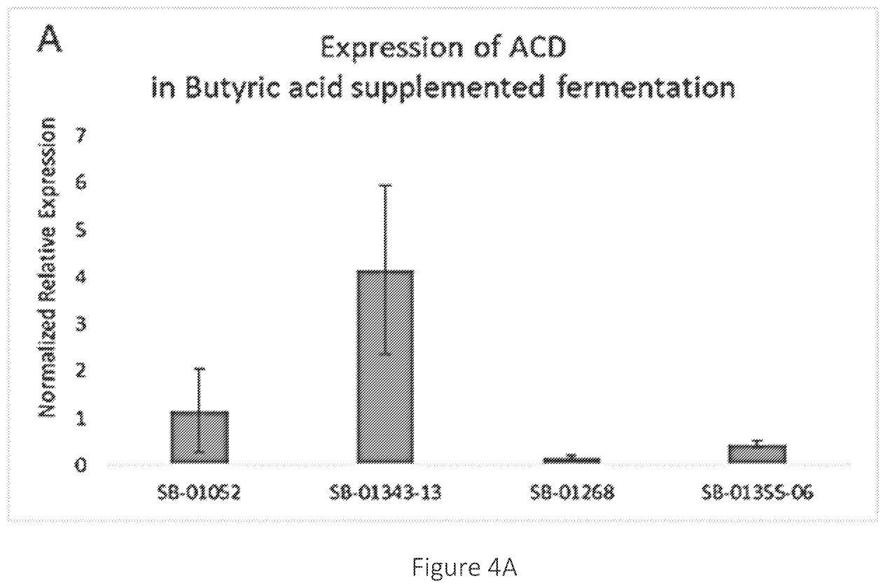

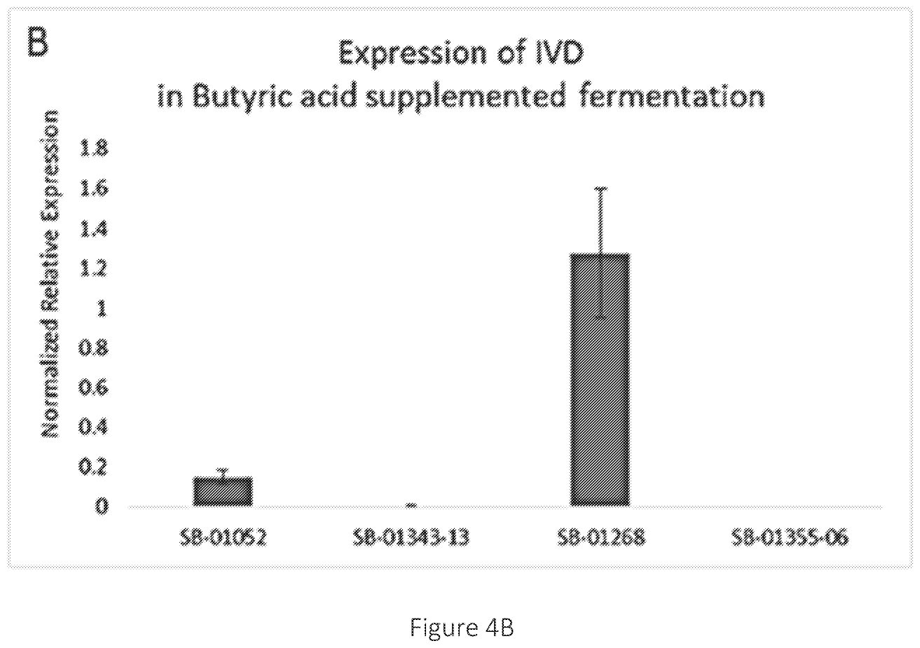

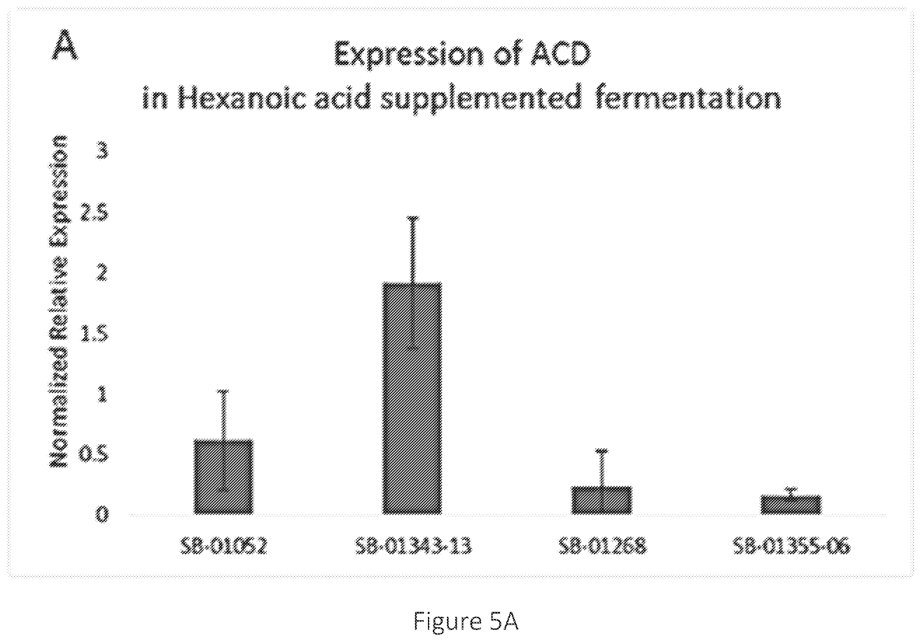

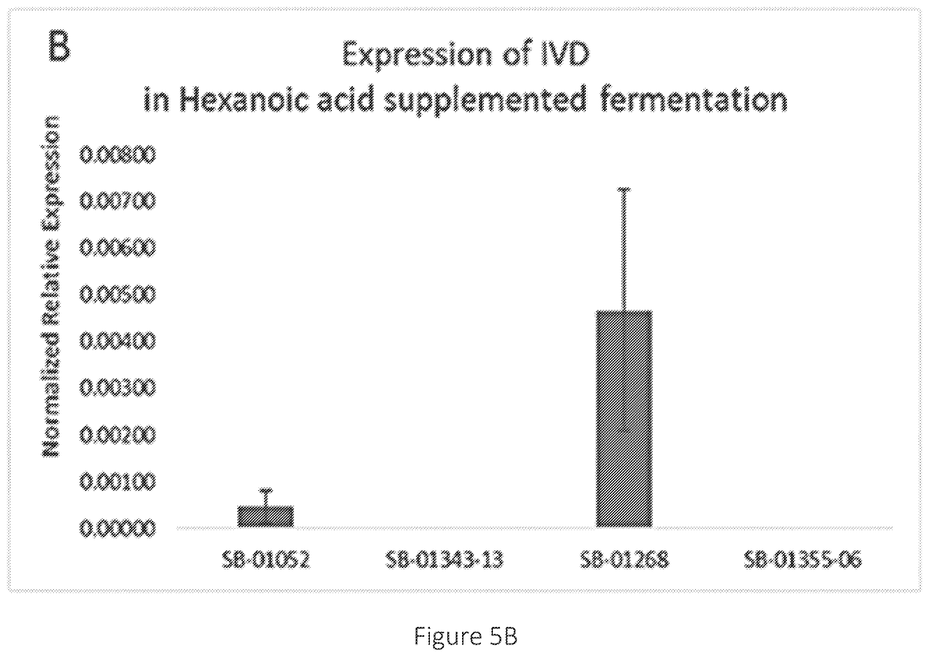

Transcriptional analysis of ACD1 and IVD1 were performed using a subset of strains from Table 34 under fermentative conditions with either butyric or hexanoic acid supplementation. The results are shown in FIGS. 4 and 5. In response to butyric acid supplementation, ACD1 showed higher expression when IVD1 was inactivated (SB-01343 has POX3, POX5, and IVD1 inactivated vs. SB-01052 has POX3, POX5 inactivated). Only base-line signal is observed in the ACD1 disruption strains, SB-01268 and SB-01355. Likewise, IVD1 showed higher expression when ACD1 was inactivated (SB-01268 has POX3, POX5, and ACD1 inactivated vs. SB-01052 has POX3, POX5 inactivated). Only base-line signal is observed in the IVD1 disruption strains, SB-01343 and SB-01355. These data show that both ACD1 and IVD1 are expressed when the cells are grown in the presence of butyric acid and that the expression of one gene is increased upon the inactivation of the other. This is consistent with these genes having redundant activity and needing to inactivate both to improve cannabinoid production from butyric or valeric acid. The same trends were observed for hexanoic acid (FIG. 5B)

### Example 11: Inactivation of Both ACD1 and IVD1 in a Strain Producing THCA

Standard molecular biology cloning techniques were used to disrupt IVD1 in SB-01542, which has ACD1 inactivated and was engineered to produce THCVA and THCA from supplemental Butyryl-CoA and hexanoyl-CoA, respectively, resulting in strain SB-01703. The genotypes of these strains are listed in Table 34.

### Example 12: Evaluating SB-01703 in Fermentation without Fatty Acid Supplementation

27 individual clones of SB-01703 along with parental strain, SB-01542 were assayed without fatty acid supplementation as describe in Example 2. The data is shown in table 10 and 11. Surprisingly, we find that our strains that contain functional genes to produce the terminal molecule, THCA, was able to produce butyryl-CoA derived cannabinoids without butyrate supplementation. Furthermore, SB-01703, appeared to have increased availability of butyryl-CoA, resulting in increased DVA yield by 2 times over SB-01542. Both SB-01542 and SB-01703 made similar amount of the terminal molecule THCVA (13 vs. 17 μM). In the same fermentation, SB-01703 also produced OA-iB (697±34 μM), this strain was able to convert OA-iB to CBGiBA and eventually to THCiBA (50 and 30 μM respectively). Whereas the parent strain SB-01542 did not produce any of these branched-chain cannabinoids. Here, we provide examples of exploiting ACD1 and IVD1 disruption to produce minor cannabinoids from sugars without external fatty acid feedstocks.

TABLE 10
C4 cannabinoid profile of IVD1 and ACD1 inactivation in our THCAS
engineered strain. Data represents an average of SB-01542 run
in triplicate and an average of 27 individual clones of SB-01703.

| | | | | | | | DVA + |
|---|---|---|---|---|---|---|---|
| | | | | | | | CBGVA + |
| | DVA | | CBGVA | | THCVA | | THCVA |
| Strain | μM | STDEV | μM | STDEV | μM | STDEV | μM |
| SB-01542 | 66 | 9 | 0 | 0 | 13 | 11 | 79 |
| SB-01703 | 134 | 43 | 13 | 8 | 17 | 8 | 164 |

TABLE 11
iC5 cannabinoid profile of IVD1 and ACD1 inactivation
in our THCAS engineered strain.

| | | | | | | | OA-iB + |
|---|---|---|---|---|---|---|---|
| | | | | | | | CBGiBA + |
| | OA-iB | | CBGiBA | | THCiBA | | THCiBA |
| Strain | μM | STDEV | μM | STDEV | μM | STDEV | μM |
| SB-01542 | 0 | 0 | 0 | 0 | 0 | 0 | 0 |
| SB-01703 | 697 | 34 | 50 | 24 | 30 | 3 | 778 |

### Example 13: Evaluating SB-01703 in Fermentation with Butyric Acid and Valeric Acid Supplementation

The above 27 individual clones of SB-01703 were assessed for their abilities to produce THCVA and THCBA using the assay method described in Example 2. Results were recorded in Table 12 and 13 for butyric acid and valeric acid supplementation. Under both feed conditions, the total C4 products (DVA, CBGVA and THCVA) and or the total of C5 products (OAB, CBGBA and THCBA) was increased by approximately four and two times, respectively, in SB-01703 (ACD1 and IVD1 inactivated) compared to SB-01542 (ACD1 inactivated only). These results indicate that, when both ACD1 and IVD1 were disrupted, more butyryl-CoA and valeryl-CoA were re-directed into our engineered pathway, increasing production of CBG(V)(B)A and THC(V)(B)A.

TABLE 12
C4 cannabinoid profile of THCAS engineered strain with ACD1 and
IVD1 inactivation during butyric acid supplemented fermentation.

| | | | | | | | DVA + |
|---|---|---|---|---|---|---|---|
| | | | | | | | CBGVA + |
| | DVA | | CBGVA | | THCVA | | THCVA |
| Strain | μM | STDEV | μM | STDEV | μM | STDEV | μM |
| SB-01542 | 642 | 112 | 21 | 4 | 77 | 25 | 740 |
| SB-01703 | 2780 | 205 | 29 | 7 | 80 | 16 | 2890 |

TABLE 13
C5 cannabinoid profile of THCAS engineered strain with ACD and
IVD inactivation during valeric acid supplemented fermentation.

| | | | | | | | OAB + |
|---|---|---|---|---|---|---|---|
| | | | | | | | CBGBA + |
| | OA-B | | CBGBA | | THCBA | | THCBA |
| Strain | μM | STDEV | μM | STDEV | μM | STDEV | μM |
| SB-01542 | 405 | 89 | 78 | 22 | 287 | 46 | 769 |
| SB-01703 | 978 | 67 | 100 | 32 | 228 | 39 | 1307 |

### Example 14: Combination of the ‘A28” Genotype and Inactivation of ACD1 Improves Cannabinoid Production

As previously reported in U.S. 63/256,398, which is incorporated herein by reference in its entirety, introduction of the ‘A28’ genotype (expression of the ERG20.A28 allele and subsequent disruption of the native ERG20 gene) results in a significant increase in GPP production. The combination of the ‘A28’ genotype and ACD1 inactivation was examined by introducing the ‘A28’ genotype into SB-01268 (ACD1 inactivated) using standard molecular biology techniques (as described in CELB-5). The resulting strain, SB-01554 was examined in 96-well deep well assays as detailed in Example 2 except that the plates were quenched at 40 h instead of 48 h (Table 14 & 15). The data indicate that combination of the ‘A28’ genotype and the disruption of ACD1 resulted in a significant improvement of both CBGA and CBGVA production compared to the ACD1 disruption alone.

TABLE 14
CBGVA produced in SB-01268 (ACD1 inactivated)
and SB-01554 (ACD1 inactivation + A28)
during butyric acid supplemented fermentation

CBGVA

| Strain | μM | STDEV |
|---|---|---|
| SB-01268 | 179.6 | 7.1 |
| SB-01554 | 500.1 | 17.1 |

TABLE 15
CBGA produced in SB-01268 (ACD1 inactivated)
and SB-01554 (ACD1 inactivation + A28)
during hexanoic acid supplemented fermentation.

CBGA

| Strain | μM | STDEV |
|---|---|---|
| SB-01268 | 627.9 | 19.2 |
| SB-01554 | 922.5 | 21.5 |

### Example 15: Combination of ACD1 Inactivation, IVD1 Inactivation and Expression of A28 Allele Improves Cannabinoid Production

To test if double inactivation of ACD1 and IVD1 in an A28 background strain (CELB-5) can further improve C4 cannabinoid, standard molecular biology techniques were employed to disrupt IVD1 gene in SB-01787 (A28 allele, POX3, POX5 and ACD1 inactivated) resulting in SB-01890. 19 individually clones of SB-01890 were screened in fermentation feeding either butyric acid, valeric acid and hexanoic acid. The average result of these 19 clones were recorded in Table 16, 17 and 18.

Under all conditions, disruption of IVD1 in SB-01787 strain background (POX3, POX5, ACD1, inactivation+A28) showed improved cannabinoids production. DVA and CBGVA produced by SB-01890 (POX3, POX5, ACD1 and IVD1 inactivated+A28) was increased by three times. OA-B and CBGBA was improved by 51%. OA and CBGA titer were improved by 34% compared to the parent strain with only ACD1 inactivation. Based on these results, inactivation of both ACD1 and IVD1 are also beneficial to improve titer in strain with engineered A28 allele background by preserving pools of butyryl-CoA, valeryl-CoA and hexanoyl-CoA and redirecting these starter CoA molecules into our engineered pathway.

TABLE 16
C4 cannabinoid profile of SB-01787 (POX3, POX5 and ACD1
inactivated and SB-01890 (POX3, POX5, ACD1 and IVD1 inactivated)
during butyric acid supplemented fermentation

| | DVA | | CBGVA | | DVA + CBGVA |
|---|---|---|---|---|---|
| Strain | μM | STDEV | μM | STDEV | μM |
| SB-01787 | 42 | 1 | 525 | 7 | 567 |
| SB-01890 | 659 | 43 | 873 | 34 | 1532 |

TABLE 17
C5 cannabinoid profile of SB-01787 (POX3, POX5 and ACD1
inactivated and SB-01890 (POX3, POX5, ACD1 and IVD1 inactivated)
during valeric acid supplemented fermentation

| | OA-B | | CBGBA | | OA-B + CBGBA |
|---|---|---|---|---|---|
| Strain | μM | STDEV | μM | STDEV | μM |
| SB-01787 | 18 | 1 | 523 | 2 | 541 |
| SB-01890 | 14 | 2 | 808 | 22 | 821 |

TABLE 18
C6 cannabinoid profile of SB-01787 (POX3, POX5 and ACD1
inactivated and SB-01890 (POX3, POX5, ACD1 and IVD1 inactivated)
during hexanoic acid supplemented fermentation.

| | | |
|---|---|---|
| OA | CBGA | OA + CBGA |

| Strain | μM | STDEV | μM | STDEV | μM |
|---|---|---|---|---|---|
| SB-01787 | 0 | 0 | 618 | 10 | 618 |
| SB-01890 | 0 | 0 | 828 | 29 | 828 |

### Example 16: Assessing the Function of POX3 and POX5 in Maintaining Improved Cannabinoid Production in DVA Producing Strain

To investigate if inactivation of POX3 and POX5 are necessary to maintain the improved DVA and CBGVA titers, POX3 and/or POX5 were disrupted in SB-02101 (ACD1 and IVD1 inactivation only) to generate SB-02403 (ACD1, IVD1 and POX5 inactivated) and SB-02404 (ACD1, IVD1, POX3 and POX5 inactivated). The resulting strains were assayed for DVA and CBGVA production with butyric acid feed fermentation as described in Example 2 and their fermentation data were recorded in Table 19 along with the parental strains. POX5 only disruption (SB-02403) showed 8% improvement of DVA and CBGVA production over the parent strain SB-02101, whereas double inactivation of POX3 and POX5 resulted in strain (SB-02404) with 58% improvement. Taken together, double POX3 and POX5 inactivation significantly increased the butyryl-CoA pool inside the cells, thus increasing our C4 cannabinoid yield. Additionally, POX3 and POX5 inactivation are synergetic with ACD1 and IVD1 disruption, suggesting that modification of both mitochondrial and peroxisomal β-oxidation genes are necessary to achieve high C4 cannabinoid titer.

TABLE 19
C4 Cannabinoid profile of CBGVA strains with
ACD1, IVD1, POX3 and POX5 inactivation during
butyric acid supplemented fermentation

| | DVA | | CBGVA | | DVA + CBGVA |
|---|---|---|---|---|---|
| Strain | μM | STDEV | μM | STDEV | μM |
| SB-02101 | 598 | 9 | 162 | 3 | 760 |
| SB-02403 | 608 | 11 | 214 | 9 | 822 |
| SB-02404 | 960 | 12 | 287 | 43 | 1197 |

### Example 17: Disruption of BAT1 and BAT2 to Reduce C5 Branched-Chain Cannabinoids

To obtain engineered strains with increased availability of butyryl-CoA and valeryl-CoA to our cannabinoid pathway without accumulation of isovaleryl-CoA, which was shown to be comparable substrates for PKS/PKC to produce the branched-chain version of cannabinoid intermediates, we sought to disrupt the enzyme upstream of IVD1 to eliminate the accumulation of isovaleryl-CoA. In S. cerevisiae, there are two functionally diverged branched-chain aminotransferases (termed BAT1 and BAT2) that are responsible for the last step of Leucine, Isoleucine and Valine biosynthesis, and the first step of the catabolism of these branched-chain amino acids (Gonzalez et al, 2011). Using the S. cerevisiae BAT1 and BAT2 sequences, we identified their homologs in Y. lipolytica to be encoded by YALI0_D01265g and YALI0_F19910g, respectively. We hypothesize that disruption of either BAT1 or BAT2 or both will prevent branched-chain amino acid catabolism (especially Leucine), halting the accumulation of isovaleryl-CoA caused by IVD1 inactivation, while preserving the benefits of increasing butyryl-CoA and valeryl-CoA flux toward our engineered pathway to produce THCVA and THCBA (when used in conjunction with ACD1 inactivation).

BAT1 and BAT2 were individually disrupted in SB-01268 (POX3, POX5 and ACD1 inactivated) to generate SB-01892 and SB-01895, respectively. In similar fashion, disruption of BAT1 and BAT2 in SB-01861 (POX3, POX5, ACD1 and IVD1) inactivated resulted in SB-01894 and SB-01897. Up to 11 clones of BAT1 and BAT2 disruption strains along with their parental strains were assayed for production of C5 branched-chain cannabinoid as described in Example 2 and the results were recorded in Table 20 and 21.

TABLE 20
iC5 cannabinoid profile of BAT1 or BAT2 and ACD1
inactivation in our cannabinoid producing strain.

| | OA-iB | | CBGiBA | | OA-iB + CBGiBA |
|---|---|---|---|---|---|
| Strain | μM | STDEV | μM | STDEV | μM |
| SB-01268 | 0 | 0 | 0 | 0 | 0 |
| SB-01892 | 0 | 0 | 0 | 0 | 0 |
| SB-01895 | 0 | 0 | 0 | 0 | 0 |

TABLE 21
iC5 cannabinoid profile of BAT1 or BAT2 inactivation,
in combination with ACD1, IVD1 inactivation
in our cannabinoid producing strain.

| | OA-iB | | CBGiBA | | OA-iB + CBGiBA |
|---|---|---|---|---|---|
| Strain | μM | STDEV | μM | STDEV | μM |
| SB-01861 | 837 | 2 | 0 | 0 | 837 |
| SB-01894 | 1080 | 31 | 0 | 0 | 1080 |
| SB-01897 | 938 | 50 | 0 | 0 | 938 |

Our result showed that disruption of BAT1 or BAT2 in SB-01268 (POX3, POX5 and ACD1 inactivated) did not change C4 or C6 cannabinoid profiles (data not shown) and did not trigger production of iC5 cannabinoids (as shown in Table 20), which strengthened our conclusion that IVD1 inactivation is needed for production of these rare branched-chain cannabinoids.

When BAT1 or BAT2 was inactivated in SB-01861 (POX3, POX5, ACD1 and IVD1 inactivated), iC5 cannabinoid production was increased by 29% and 12%, respectively. This result implied that when BAT1 or BAT2 were disrupted, there is an increased buildup of isovaleryl-CoA inside the cell, causing increase in iC5 cannabinoid production. Thus, contrary to our initial hypothesis, deleting BAT1 and/or BAT2 is a way to increase branch chain cannabinoids.

### Example 18: Inactivation of BCKD-E1α, E1β, or E2 Subunit to Reduce C5 Branched-Chain Cannabinoids

Another approach to reduce accumulation of isovaleryl-CoA is to inactivate the branched-chain α-keto-acid dehydrogenase complex (BCKD) which is involved in generating isovaleryl-CoA from α-ketoisocaproate (FIG. 3). This complex is made up of four subunits, Ela (YALI0_D08690g), E1β (YALI0_F05038g), E2 (YALI0_D23815g) and E3 (YALI0D20768g). E3 subunit is shared with the Pyruvate dehydrogenase and Alpha-ketoglutarate dehydrogenase complexes. Thus, to specifically inactivate BCKD, subunit Ela, E1β, or E2 can be disrupted. These genes were inactivated in SB-02218 (POX3, POX5, ACD1 and IVD1 inactivated) to generate SB-02225, SB-02226 and SB-02227, respectively. 11 clones for each disruption strain were assayed for C4 and iC5 cannabinoid production as described in Example 2. The data were recorded in Table 22 and 23.

Disruption of BCKD-E1α (SB-02225) or E2 subunit (SB-02227) significantly reduced iC5 products by almost 8-fold compared to the parent SB-02218, whereas disruption of BCKD-E1β did not reduce C5 branched-chain cannabinoids. Furthermore, strains with BCKD-E1α or BCKD-E2 inactivated surprisingly also had significant increase in CBGVA production, converting more than 95% of DVA, produced from butyric acid feed, to CBGVA and THCVA, this result suggests that in addition to reducing the iC5 products, disruption of BCKD-E1α or BCKD-E2 also results in (1) more GPP being available for conversion of DVA to CBGVA and/or (2) alleviating substrate competition of CBGA synthase by C5 branched-chain products.

TABLE 22
iC5 cannabinoid profile of BCKD-E1α, E1β, E2
subunit inactivation together with POX3, POX5, ACD1
and IVD1 inactivation in our THCAS engineered strain

| | | | | | | | OA-iB + |
|---|---|---|---|---|---|---|---|
| | | | | | | | CBGiBA + |
| | OA-iB | | CBGiBA | | THCiBA | | THCiBA |
| Strain | μM | STDEV | μM | STDEV | μM | STDEV | μM |
| SB-02218 | 387 | 62 | 425 | 34 | 75 | 12 | 887 |
| SB-02225 | 0 | 0 | 101 | 2 | 17 | 1 | 118 |
| SB-02226 | 415 | 89 | 400 | 45 | 75 | 18 | 890 |
| SB-02227 | 0 | 0 | 102 | 4 | 22 | 1 | 124 |

TABLE 23
C4 cannabinoid profile of BCKD-E1α, E1β, E2 subunit inactivation
together with POX3, POX5, ACD1 and IVD1 inactivation in our
THCAS engineered strain during butyric acid supplementation

| | | | | | | | DVA + |
|---|---|---|---|---|---|---|---|
| | | | | | | | CBGVA + |
| | DVA | | CBGVA | | THCVA | | THCVA |
| Strain | μM | STDEV | μM | STDEV | μM | STDEV | μM |
| SB-02218 | 710 | 63 | 343 | 19 | 333 | 49 | 1386 |
| SB-02225 | 32 | 5 | 934 | 25 | 303 | 42 | 1269 |
| SB-02226 | 732 | 93 | 320 | 43 | 320 | 75 | 1372 |
| SB-02227 | 63 | 49 | 766 | 3 | 390 | 71 | 1220 |

### Example 19: Inactivation of ARO10 to Reduce C5 Branched-Chain Cannabinoids

As seen in FIG. 3, the first step in the degradation of Leucine produces α-ketoisocaproate. It is possible that this compound is decarboxylated in the cytosol by a keto-acid decarboxylase (part of the Ehrlich pathway). The resulting isoamylaldehyde could be oxidized to isovalerate which could then be utilized by the cannabinoid synthesis pathway in our organism. To eliminate this possible route to isovalerate, the keto-acid decarboxylase (ARO10, YALI0_D06930g) can be disrupted. To test this hypothesis, this gene was inactivated in (SB-02218 POX3, POX5, ACD1 and IVD1 inactivated) and assayed for cannabinoid production as described in Example 2.

### Example 20: Inactivation of Peroxisomal Acyl-CoA Oxidase Genes (POX1, POX2, POX4, POX6) in Combination with ACD1, POX3, POX5 Inactivation and A28 Allele Improves Cannabinoid Production

Acyl-CoA oxidase is the first committed step in peroxisomal 3-oxidation which is a competing pathway for hexanoyl-CoA and butyryl-CoA molecules. Hence, we sought to increase cannabinoid product titers by inactivating peroxisomal β-oxidation by individually disrupting acyl-CoA genes including: POX1 (YALI0_E32835g), POX2 (YALI0_F10857g), POX4 (YALI0_E27654g), or POX6 (YALI0_E06567g). These individual gene disruptions were conducted in strain background SB-01983 which contains the A28 allele and is inactivated for ACD1, POX3, and POX5 genes. The resulting engineered strains (SB-02257, ΔPOX1; SB-02258, ΔPOX2; SB-02259, ΔPOX4; SB-02265, ΔPOX6) were assayed in quadruplicate for cannabinoid production as described in Example 2. The data in Table 24 confirmed that disruption of POX2, or POX6 in combination with the A28 allele and inactivation of ACD1, POX3 and POX5 significantly improved cannabinoid product titers over the background strain SB-01983 while disruption of POX1 or POX4 had no significant improvement in product titers.

TABLE 24
Cannabinoid profiles produced by engineered strains
inactivated for peroxisomal acyl-CoA oxidase genes
supplemented with hexanoic acid. Data represents the
average and standard deviation between 4 replicates.

| | | | | | | | OA + |
|---|---|---|---|---|---|---|---|
| | | | | | | | CBGA + |
| | OA | | CBGA | | THCA | | THCA |
| Strain | μM | STDEV | μM | STDEV | μM | STDEV | μM |
| SB-01983 | 51 | 24 | 283 | 71 | 377 | 68 | 712 |
| SB-02257 | 0 | 0 | 201 | 46 | 453 | 19 | 653 |
| SB-02258 | 8 | 0 | 633 | 90 | 511 | 115 | 1152 |
| SB-02259 | 6 | 0 | 340 | 94 | 381 | 78 | 727 |
| SB-02265 | 9 | 1 | 1060 | 33 | 0 | 0 | 1069 |

### Example 21: Inactivation of ACD Homologues (GCDH1, GCDH2, ACD2) in Combination with ACD1, POX3, POX5 Inactivation and A28 Allele Improves Cannabinoid Production

Mitochondrial β-oxidation is another competing pathway for hexanoyl-CoA and butyryl-CoA molecules. Since inactivation of mitochondrial β-oxidation via ACD1 disruption increased product titers, we sought to further increase product titers by also disrupting the ACD1 homologues identified by BLAST search (see Example 5, Table 6). To test this, we individually disrupted GCDH1 (YALI0_C16797g), GCDH2 (YALI0_F23749g), or ACD2 (YALI0_B04906g) in strain SB-01983 (A28 allele, inactivated for ACD1, POX3, and POX5). The resulting engineered strains (SB-02260, ΔGCDH1; SB-02261, ΔGCDH2; SB-02262, ΔACD2) were assayed in quadruplicate for cannabinoid production as described in Example 2. The data in Table 25 confirmed that disruption of GCDH2 in combination with the A28 allele and inactivation of ACD1, POX3 and POX5 improved cannabinoid product titers over the background strain SB-01983 by 16%, however, disruption of GCDH1 or ACD2 showed no significant improvements.

TABLE 25
Cannabinoid profiles produced by engineered strains
inactivated for ACD homologue genes supplemented
with hexanoic acid. Data represents the average
and standard deviation between 4 replicates.

| | | | | | | | OA + |
|---|---|---|---|---|---|---|---|
| | | | | | | | CBGA + |
| | OA | | CBGA | | THCA | | THCA |
| Strain | μM | STDEV | μM | STDEV | μM | STDEV | μM |
| SB-01983 | 51 | 24 | 283 | 71 | 377 | 68 | 712 |
| SB-02260 | 10 | 11 | 489 | 119 | 286 | 112 | 785 |
| SB-02261 | 7 | 0 | 380 | 62 | 438 | 53 | 824 |
| SB-02262 | 7 | 1 | 330 | 93 | 382 | 104 | 719 |

### Example 22: Inactivation of Peroxisome Biogenesis Through PEX10 in Combination with ACD1, POX3, POX5 Inactivation and A28 Allele Improves Cannabinoid Production

Since peroxisomal β-oxidation is a competing pathway for hexanoyl-CoA and butyryl-CoA molecules, we sought to abolish peroxisome biogenesis to inhibit the peroxisomal β-oxidation pathway. To accomplish this, we inactivated peroxisomal biogenesis factor 10 (PEX10, YALI0_D02387g) in strain background SB-01983 which contains the A28 allele and is inactivated for ACD1, POX3, and POX5 genes. The resulting engineered strain (SB-02264) was assayed in quadruplicate for cannabinoid production as described in Example 2. The data in Table 26 confirmed that disruption of PEX10 in combination with the A28 allele and inactivation of ACD1, POX3 and POX5 significantly increased cannabinoid product titers by 41% over the background strain SB-01983.

TABLE 26
Cannabinoid profiles produced by engineered strain inactivated
for peroxisomal biogenesis (by disruption of PEX10) supplemented
with hexanoic acid. Data represents the average and standard
deviation between 4 replicates.

| | | | | | | | OA + |
|---|---|---|---|---|---|---|---|
| | | | | | | | CBGA + |
| | OA | | CBGA | | THCA | | THCA |
| Strain | μM | STDEV | μM | STDEV | μM | STDEV | μM |
| SB-01983 | 51 | 24 | 283 | 71 | 377 | 68 | 712 |
| SB-02264 | 614 | 81 | 115 | 48 | 276 | 21 | 1005 |

### Example 23: Inactivation of Multifunctional Beta-Oxidation Enzyme (MFE1) in Combination with ACD1, POX3, POX5 Inactivation and A28 Allele Improves Cannabinoid Production

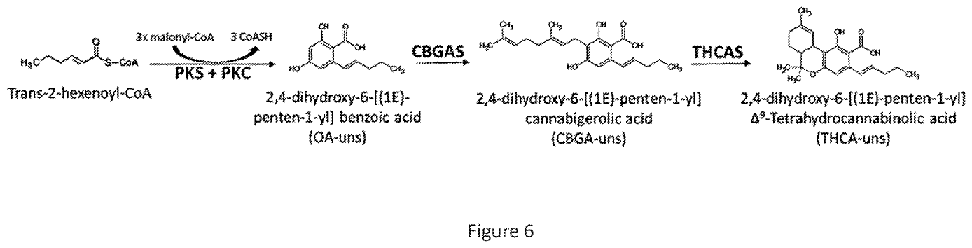

The second and third step of peroxisomal β-oxidation is accomplished by multifunctional beta-oxidation enzyme hydratase-dehydrogenase-epimerase (MFE1, YALI0_E15378G) (Beopoulos et al., 2008). Hence, this gene is responsible for converting 2-trans-enoyl CoA molecules into L-3-hydroxy acyl CoA and sequentially to 3-ketoacyl CoA as seen in FIG. 2. Inactivation of MFE1 was conducted in strain background SB-01983 which contains the A28 allele and is inactivated for ACD1, POX3, and POX5. The resulting engineered strain SB-02263 also produces rare cannabinoids in which the hexanoyl-CoA molecule is replaced with trans-2-hexenoyl-CoA, which was confirmed by LC-MS (FIG. 6). The data in Table 27 confirmed that disruption of MFE1 in combination with the A28 allele and inactivation of ACD1, POX3 and POX5 significantly increased cannabinoid product titers over the background strain SB-01983. The data in Table 28 confirmed that disruption of MFE1 in combination with the A28 allele and inactivation of ACD1, POX3 and POX5 produced rare cannabinoids in which the hexanoyl-CoA molecule is replaced with trans-2-hexenoyl-CoA. Together, these results show that inactivation of peroxisomal β-oxidation pathway by disrupting MFE1 not only increases cannabinoid product titers but also produces rare cannabinoid species.

TABLE 27
Cannabinoid profiles produced by engineered strain inactivated
for peroxisomal β-oxidation steps (by disruption of
MFE1) supplemented with hexanoic acid. Data represents
the average and standard deviation between 4 replicates.

| | | | | | | | OA + |
|---|---|---|---|---|---|---|---|
| | | | | | | | CBGA + |
| | OA | | CBGA | | THCA | | THCA |
| Strain | μM | STDEV | μM | STDEV | μM | STDEV | μM |
| SB-01983 | 51 | 24 | 283 | 71 | 377 | 68 | 712 |
| SB-02263 | 196 | 21 | 286 | 12 | 588 | 4 | 1071 |

TABLE 28
Rare cannabinoid profiles (where the hexanoyl-CoA molecule is
replaced with trans-2-hexenoyl-CoA) produced by engineered
strain inactivated for peroxisomal β-oxidation steps (by
disruption of MFE1) supplemented with hexanoic acid. Data represents
the average and standard deviation between 2 replicates.

| | | | | | | | OA-uns + |
|---|---|---|---|---|---|---|---|
| | | | | | | | CBGA-uns + |
| | OA-uns | | CBGA-uns | | THCA-uns | | THCA-uns |
| Strain | μM | STDEV | μM | STDEV | μM | STDEV | μM |
| SB-01983 | 0 | 0 | 0 | 0 | 0 | 0 | 0 |
| SB-02263 | 88 | 2 | 54 | 7 | 93 | 5 | 235 |

### Example 24: Assessing Disruption of POT1 to Reduce Beta Oxidation and Improve Cannabinoid Production

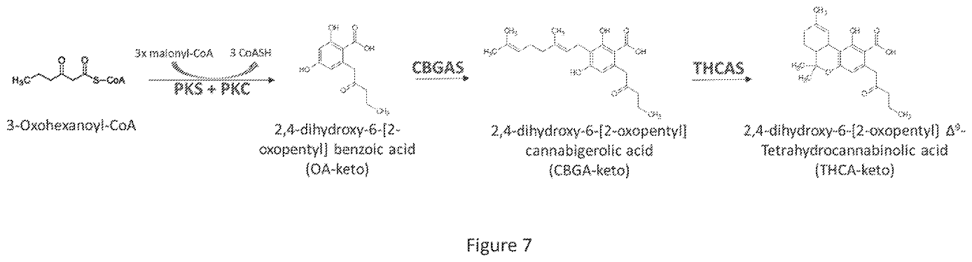

The fourth and final step in peroxisomal β-oxidation is accomplished by peroxisomal oxoacyl thiolase (POT1, YALI0_E18568g) (Beopoulos et al., 2008). Hence, inactivation of POT1 should inhibit this pathway from degrading hexanoyl-CoA or butyryl-CoA to acetyl-CoA. Inactivation of POT1 could also enable production of rare ketolated cannabinoid species in which the hexanoyl-CoA molecule is replaced by 3-oxohexanoyl-CoA since POT1 is needed to convert ketolated beta oxidation products into acyl-CoA molecules (FIG. 7). POT1 will be disrupted in SB-01983 (POX3, POX5, and ACD1 inactivated, and with A28 allele). The resulting strains will be assayed for C4 and C6 cannabinoid production and production of rare ketolated cannabinoids as described in Example 2.

### Example 25: Assessing Disruption of ANT1 and FAA1 to Inhibit Fatty Acid Activation to Improve Cannabinoid Production

Fatty acids (e.g., hexanoic acid, butyric acid) are activated by reacting with CoA molecules to form fatty acyl-CoA. Fatty acids are activated in the cytosol by a long-chain fatty-acid CoA ligase (FAA1) (YALI0_D17864g) or in the peroxisome by enzymes that require ATP generated by peroxisomal adenine nucleotide transporter 1 (ANT1) (YALI0_E03058g) (Dulermo et al., 2015). Activated fatty acids are either stored in lipid bodies or are degraded by beta oxidation. Therefore, disruption of FAA1 and/or ANT1 will inhibit fatty acid activation and block their degradation by beta oxidation or storage in lipid bodies to improve cannabinoid production. To test this, we individually disrupted FAA1 or ANT1 in strain SB-01983 (POX3, POX5, and ACD1 inactivated, and with A28 allele). The resulting engineered strains (SB-02456, ΔFAA1; SB-02500, ΔANT1) were assayed for cannabinoid production as described in Example 2. Disruption of FAA1 (SB-02456) in combination with the A28 allele and inactivation of ACD1, POX3 and POX5 significantly improved C4 cannabinoid product titers over the background strain SB-01983 with both hexanoic acid (Table 29) or butyric acid (Table 30) supplementation while no improvement was observed in C6 cannabinoid product titers. Disruption of ANT1 (SB-02500) in combination with the A28 allele and inactivation of ACD1, POX3 and POX5 improved C6 cannabinoid production while having no impact on C4 cannabinoid titers (Tables 29 and 30).

TABLE 29
Cannabinoid profiles produced by engineered strains disrupted
for fatty acid activation genes (FAA1 or ANT1) supplemented
with hexanoic acid. Data represents the average and standard
deviation between 3 replicates.

| | | | CBGA + | | | | CBGVA + | |
|---|---|---|---|---|---|---|---|---|
| | OA | | THCA | | DVA | | THCVA | |
| Strain | μM | STDEV | μM | STDEV | μM | STDEV | μM | STDEV |
| SB- | 0 | 0 | 561 | 22 | 35 | 1 | 60 | 5 |
| 01983 | | | | | | | | |
| SB- | 21 | 33 | 562 | 174 | 596 | 74 | 242 | 69 |
| 02456 | | | | | | | | |
| SB- | 3 | 6 | 716 | 126 | 35 | 10 | 60 | 18 |
| 02500 | | | | | | | | |

TABLE 30
Cannabinoid profiles produced by engineered strains disrupted
for fatty acid activation genes (FAA1 or ANT1) supplemented
with butyric acid. Data represents the average and standard
deviation between 3 replicates.

| | |
|---|---|
| DVA | CBGVA + THCVA |

| Strain | μM | STDEV | μM | STDEV |
|---|---|---|---|---|
| SB-01983 | 61 | 7 | 311 | 23 |
| SB-02456 | 412 | 112 | 647 | 218 |
| SB-02500 | 51 | 26 | 262 | 144 |

### Example 26: Assessing Disruption of YAT1 to Reduce Beta Oxidation and to Improve Cannabinoid Production

The activated hexanoyl-CoA and butyryl-CoA in the cytosol can be shuttled into the peroxisome and mitochondria to be metabolized by beta oxidation. Cytosolic carnitine acetyltransferase YAT1 (YALI0_F21197g) may be attaching carnitine to hexanoyl-CoA or butyryl-CoA. To test this, we inactivated YAT1 in strain background SB-01983 which contains the A28 allele and is inactivated for ACD1, POX3, and POX5 genes. The resulting engineered strain (SB-02457) was assayed in triplicate for cannabinoid production as described in Example 2. The data in Table 31 confirmed that disruption of YAT1 in combination with the A28 allele and inactivation of ACD1, POX3 and POX5 increased cannabinoid product titers over the background strain SB-01983.

TABLE 31
Cannabinoid profiles produced by engineered strain inactivated
for cytosolic carnitine acetyltransferase (YAT1) supplemented
with hexanoic acid. Data represents the average and
standard deviation between 3 replicates.

| | | | | | | | OA + |
|---|---|---|---|---|---|---|---|
| | | | | | | | CBGA + |
| | OA | | CBGA | | THCA | | THCA |
| Strain | μM | STDEV | μM | STDEV | μM | STDEV | μM |
| SB-01983 | 0 | 0 | 341 | 163 | 364 | 54 | 705 |
| SB-02457 | 14 | 24 | 252 | 41 | 527 | 57 | 793 |

### Example 27: Inactivation of FAA1, ACD1, IVD1 and BCKD to Further Improve Cannabinoid Production

In previous examples, disruption of either IVD1 (with or without BCKD inactivation) or FAA1 increases C4 cannabinoid product titers. To test if the positive improvement of IVD and FAA1 disruptions are synergistic and additive, we sought to disrupt FAA1 in strain SB-02762 that harbors deletion of POX3, POX5, ACD1, IVD1 and BCKD-E1α in combination with the A28 allele. The resulting engineered strain (SB-03603) was assayed in triplicate for cannabinoid production as described in Example 2. SB-03603 significantly improved C4 cannabinoid product titers over the background strain (SB-02762) without any supplementation (Table 32) and with butyric acid supplementation (Table 33).

TABLE 32
Cannabinoid profiles produced by engineered strain disrupted
for FAA1 (SB-03603) without any supplementation. Data represents
the average and standard deviation between 3 replicates.

| | | | | | | | DVA + CBGVA + | |
|---|---|---|---|---|---|---|---|---|
| | DVA | | CBGVA | | THCVA | | THCVA | |
| Strain | μM | STDEV | μM | STDEV | μM | STDEV | μM | STDEV |
| SB-02762 | 0 | 0 | 56 | 2 | 70 | 8 | 126 | 9 |
| SB-03603 | 1347 | 119 | 1796 | 68 | 716 | 13 | 3859 | 93 |

TABLE 33
Cannabinoid profiles produced by engineered strain disrupted for
FAA1 (SB-03603) supplemented with butyric acid. Data represents
the average and standard deviation between 3 replicates.

| | | | | | | | DVA + CBGVA + | |
|---|---|---|---|---|---|---|---|---|
| | DVA | | CBGVA | | THCVA | | THCVA | |
| Strain | μM | STDEV | μM | STDEV | μM | STDEV | μM | STDEV |
| SB-02762 | 194 | 114 | 482 | 39 | 494 | 70 | 1170 | 33 |
| SB-03603 | 3111 | 539 | 1579 | 291 | 535 | 32 | 5226 | 251 |

### Example 28: Inactivation of Just FAA1 to Further Improve Cannabinoid Production

In previous examples, disruption of FAA1 increases C4 cannabinoid production with or without butyric acid supplementation. To test if FAA1 inactivation for improvement of C4 cannabinoid production is dependent on other gene disruptions (ACD, POX3, POX5, IVD, BCKD-E3a), we sought to inactivate FAA1 in strain SB-03715 which harbors no other gene disruptions nor the A28 allele. The resulting strain will be assayed for C4 cannabinoid production as previously described in Example 2.

TABLE 34
A summary of strains constructed and utilized in the instant examples.

| Strain | Key gene(s) expressed | Gene(s) inactivated |
|---|---|---|
| SB-00889 | HCS2, PKS1, PKC1.1, HMGR, | |
| | ERG20WW, ERG20WW-PKC1.1- | |
| | MPT4, ACS1 | |
| SB-01052 | HCS2, PKS1, PKC1.1, HMGR, | POX3, POX5 |
| | ERG20WW, ERG20WW-PKC1.1- | |
| | MPT4, ACS1 | |
| SB-01209 | HCS2, PKS1, PKC1.1, HMGR, | POX3, POX5, ACD1 |
| | ERG20WW, ERG20WW-PKC1.1- | |
| | MPT4, ACS1 | |
| SB-01211 | HCS2, PKS1, PKC1.1, HMGR, | POX3, POX5, ECH1 |
| | ERG20WW, ERG20WW-PKC1.1- | |
| | MPT4, ACS1 | |
| SB-01213 | HCS2, PKS1, PKC1.1, HMGR, | ACD1 |
| | ERG20WW, ERG20WW-PKC1.1- | |
| | MPT4, ACS1 | |
| SB-01215 | HCS2, PKS1, PKC1.1, HMGR, | ECH1 |
| | ERG20WW, ERG20WW-PKC1.1- | |
| | MPT4, ACS1 | |
| SB-01268 | HCS2, PKS1, PKC1.1, HMGR, | POX3, POX5, ACD1 |
| | ERG20WW, ERG20WW-PKC1.1- | |
| | MPT4, ACS1 | |
| SB-01343 | HCS2, PKS1, PKC1.1, HMGR, | POX3, POX5, IVD1 |
| | ERG20WW, ERG20WW-PKC1.1- | |
| | MPT4, ACS1 | |
| SB-01355 | HCS2, PKS1, PKC1.1, HMGR, | POX3, POX5, ACD1, IVD1 |
| | ERG20WW, ERG20WW-PKC1.1- | |
| | MPT4, ACS1 | |
| SB-01542 | HCS2, PKS1, PKC1.1, HMGR, | POX3, POX5, ACD1 |
| | ERG20WW, ERG20WW-PKC1.1- | |
| | MPT4, ACS1, CNE1, THCAS | |
| SB-01691 | HCS2, PKS1.1, PKC1, ERG20WW | ERG20, ACD1 |
| SB-01703 | HCS2, PKS1, PKC1.1, HMGR, | POX3, POX5, ACD1, IVD1 |
| | ERG20WW, ERG20WW-PKC1.1- | |
| | MPT4, ACS1, CNE1, THCAS | |
| SB-01787 | HCS2, PKS1, PKC1.1, HMGR, | ERG20, POX3, POX5, ACD1 |
| | ERG20WW, ERG20WW-PKC1.1- | |
| | MPT4, ACS1, ERG20WW-PKC1.1- | |
| | MPT4.1 | |
| SB-01861 | HCS2, PKS1, PKC1.1, HMGR, | POX3, POX5, ACD1, IVD1 |
| | ERG20WW, ERG20WW-PKC1.1- | |
| | MPT4, ACS1 | |
| SB-01890 | HCS2, PKS1, PKC1.1, HMGR, | ERG20, POX3, POX5, ACD1, IVD1 |
| | ERG20WW, ERG20WW-PKC1.1- | |
| | MPT4, ACS1, ERG20WW-PKC1.1- | |
| | MPT4.1 | |
| SB-01892 | HCS2, PKS1, PKC1.1, HMGR, | POX3, POX5, ACD1, BAT1 |
| | ERG20WW, ERG20WW-PKC1.1- | |
| | MPT4, ACS1 | |
| SB-01894 | HCS2, PKS1, PKC1.1, HMGR, | POX3, POX5, ACD1, IVD, BAT1 |
| | ERG20WW, ERG20WW-PKC1.1- | |
| | MPT4, ACS1 | |
| SB-01895 | HCS2, PKS1, PKC1.1, HMGR, | POX3, POX5, ACD1, BAT2 |
| | ERG20WW, ERG20WW-PKC1.1- | |
| | MPT4, ACS1 | |
| SB-01897 | HCS2, PKS1, PKC1.1, HMGR, | POX3, POX5, ACD1, IVD1, BAT2 |
| | ERG20WW, ERG20WW-PKC1.1- | |
| | MPT4, ACS1 | |
| SB-01983 | HCS2, PKS1, PKC1.1, HMGR, | ERG20, POX3, POX5, ACD1 |
| | ERG20WW, ERG20WW-PKC1.1- | |
| | MPT4, ACS1, ERG20WW-PKC1.1- | |
| | MPT4.1, CNE1, THCAS | |
| SB-02101 | HCS2, PKS1.1, PKC1, ERG20WW | ERG20, ACD1, IVD1 |
| SB-02218 | HCS2, PKS1, PKC1.1, HMGR, | ERG20, POX3, POX5, ACD1, IVD1 |
| | ERG20WW, ERG20WW-PKC1.1- | |
| | MPT4, ACS1, ERG20WW-PKC1.1- | |
| | MPT4.1, CNE1, THCAS | |
| SB-02225 | HCS2, PKS1, PKC1.1, HMGR, | ERG20, POX3, POX5, ACD1, IVD1, |
| | ERG20WW, ERG20WW-PKC1.1- | BCKD-E1α |
| | MPT4, ACS1, ERG20WW-PKC1.1- | |
| | MPT4.1, CNE1, THCAS | |
| SB-02226 | HCS2, PKS1, PKC1.1, HMGR, | ERG20, POX3, POX5, ACD1, IVD1, |
| | ERG20WW, ERG20WW-PKC1.1- | BCKD-E1β |
| | MPT4, ACS1, ERG20WW-PKC1.1- | |
| | MPT4.1, CNE1, THCAS | |
| SB-02227 | HCS2, PKS1, PKC1.1, HMGR, | ERG20, POX3, POX5, ACD1, IVD1, |
| | ERG20WW, ERG20WW-PKC1.1- | BCKD-E2 |
| | MPT4, ACS1, ERG20WW-PKC1.1- | |
| | MPT4.1, CNE1, THCAS | |
| SB-02257 | HCS2, PKS1, PKC1.1, HMGR, | ERG20, POX3, POX5, ACD1, POX1 |
| | ERG20WW, ERG20WW-PKC1.1- | |
| | MPT4, ACS1, ERG20WW-PKC1.1- | |
| | MPT4.1, CNE1, THCAS | |
| SB-02258 | HCS2, PKS1, PKC1.1, HMGR, | ERG20, POX3, POX5, ACD1, POX2 |
| | ERG20WW, ERG20WW-PKC1.1- | |
| | MPT4, ACS1, ERG20WW-PKC1.1- | |
| | MPT4.1, CNE1, THCAS | |
| SB-02259 | HCS2, PKS1, PKC1.1, HMGR, | ERG20, POX3, POX5, ACD1, POX4 |
| | ERG20WW, ERG20WW-PKC1.1- | |
| | MPT4, ACS1, ERG20WW-PKC1.1- | |
| | MPT4.1, CNE1, THCAS | |
| SB-02260 | HCS2, PKS1, PKC1.1, HMGR, | ERG20, POX3, POX5, ACD1, |
| | ERG20WW, ERG20WW-PKC1.1- | GCDH1 |
| | MPT4, ACS1, ERG20WW-PKC1.1- | |
| | MPT4.1, CNE1, THCAS | |
| SB-02261 | HCS2, PKS1, PKC1.1, HMGR, | ERG20, POX3, POX5, ACD, GCDH2 |
| | ERG20WW, ERG20WW-PKC1.1- | |
| | MPT4, ACS1, ERG20WW-PKC1.1- | |
| | MPT4.1, CNE1, THCAS | |
| SB-02262 | HCS2, PKS1, PKC1.1, HMGR, | ERG20, POX3, POX5, ACD1, ACD2 |
| | ERG20WW, ERG20WW-PKC1.1- | |
| | MPT4, ACS1, ERG20WW-PKC1.1- | |
| | MPT4.1, CNE1, THCAS | |
| SB-02263 | HCS2, PKS1, PKC1.1, HMGR, | ERG20, POX3, POX5, ACD1, MFE1 |
| | ERG20WW, ERG20WW-PKC1.1- | |
| | MPT4, ACS1, ERG20WW-PKC1.1- | |
| | MPT4.1, CNE1, THCAS | |
| SB-02264 | HCS2, PKS1, PKC1.1, HMGR, | ERG20, POX3, POX5, ACD1, PEX10 |
| | ERG20WW, ERG20WW-PKC1.1- | |
| | MPT4, ACS1, ERG20WW-PKC1.1- | |
| | MPT4.1, CNE1, THCAS | |
| SB-02265 | HCS2, PKS1, PKC1.1, HMGR, | ERG20, POX3, POX5, ACD1, POX6 |
| | ERG20WW, ERG20WW-PKC1.1- | |
| | MPT4, ACS1, ERG20WW-PKC1.1- | |
| | MPT4.1, CNE1, THCAS | |
| SB-02351 | HCS2, PKS1, PKC1.1, HMGR, | ERG20, POX3, POX5, ACD1, IVD1, |
| | ERG20WW, ERG20WW-PKC1.1- | BCKD-E1α |
| | MPT4, ACS1, ERG20WW-PKC1.1- | |
| | MPT4.1, CNE1, THCAS | |
| SB-02403 | HCS2, PKS1.1, PKC1, ERG20WW | ERG20, ACD1, IVD1, POX5 |
| SB-02404 | HCS2, PKS1.1, PKC1, ERG20WW | ERG20, ACD1, IVD1, POX5, POX3 |
| SB-02456 | HCS2, PKS1, PKC1.1, HMGR, | ERG20, POX3, POX5, ACD1, FAA1 |
| | ERG20WW, ERG20WW-PKC1.1- | |
| | MPT4, ACS1, ERG20WW-PKC1.1- | |
| | MPT4.1, CNE1, THCAS | |
| SB-02457 | HCS2, PKS1, PKC1.1, HMGR, | ERG20, POX3, POX5, ACD1, YAT1 |
| | ERG20WW, ERG20WW-PKC1.1- | |
| | MPT4, ACS1, ERG20WW-PKC1.1- | |
| | MPT4.1, CNE1, THCAS | |
| SB-02500 | HCS2, PKS1, PKC1.1, HMGR, | ERG20, POX3, POX5, ACD1, ANT1 |
| | ERG20WW, ERG20WW-PKC1.1- | |
| | MPT4, ACS1, ERG20WW-PKC1.1- | |
| | MPT4.1, CNE1, THCAS | |
| SB-02762 | HCS2, PKS1, PKC1.1, HMGR, | ERG20, POX3, POX5, ACD1, IVD1, |
| | ERG20WW, ERG20WW-PKC1.1- | BCKD-E1α |
| | MPT4, ACS1, ERG20WW-PKC1.1- | |
| | MPT4.1, CNE1, THCAS | |
| SB-03175 | HCS2, PKS1.1, PKC1, ERG20WW | |
| SB-03603 | HCS2, PKS1, PKC1.1, HMGR, | ERG20, POX3, POX5, ACD1, IVD1, |
| | ERG20WW, ERG20WW-PKC1.1- | BCKD-E1α, FAA1 |
| | MPT4, ACS1, ERG20WW-PKC1.1- | |
| | MPT4.1, CNE1, THCAS | |

1.-39. (canceled)

40. A cell engineered to provide increased production of cannabinoids and/or derivatives thereof compared to a control cell, the engineered cell expressing one or more enzymes of the cannabinoid biosynthesis pathway,
wherein at least one gene encoding one or more enzymes or other proteins influencing metabolism of at least one acyl-CoA molecule of the cell has been deleted, inactivated, or otherwise mutated, thereby permitting the redirection of acyl-CoA molecules from a cellular metabolic pathway to a cannabinoid biosynthesis pathway to achieve increased production of cannabinoids and/or derivatives thereof compared to a control cell, and
wherein the control cell is lacking the same deletion, inactivation or other mutation of the at least one gene as the engineered cell.

41. The cell of claim 40, wherein the cellular metabolic pathway is a beta-oxidation pathway, optionally, wherein the beta-oxidation pathway is one or both of a mitochondrial beta-oxidation pathway and/or a peroxisomal beta-oxidation pathway.

42. The cell of claim 40, wherein the cellular metabolic pathway is an amino acid catabolic pathway, optionally, wherein the amino acid catabolic pathway is one or more branched chain amino acid catabolic pathway selected from the group consisting of a leucine, an isoleucine and/or a valine catabolic pathway which produces a branched acyl-CoA intermediate.

43. The cell of claim 40, wherein the acyl-CoA comprises one or more of butyryl-CoA, valeryl-CoA, isovaleryl-CoA, trans-2-hexenoyl-CoA, 3-oxohexanoyl-CoA and/or hexanoyl-CoA.

44. The cell of claim 40, wherein the at least one gene encodes a peroxisomal acyl-CoA oxidase, a peroxisomal acyl-CoA dehydrogenase, a mitochondrial acyl-CoA dehydrogenase, a mitochondrial isovaleryl-CoA dehydrogenase, a mitochondrial branched-chain α-keto acid dehydrogenase subunit, an isovaleryl-CoA dehydrogenase, a branched-chain aminotransferase, a glutaryl-CoA dehydrogenase, a keto-acid decarboxylase, a multifunctional beta-oxidation enzyme hydratase-dehydrogenase-epimerase, a peroxisomal oxoacyl thiolase, fatty-acid CoA ligase, a peroxisomal adenine nucleotide transporter, a cytosolic carnitine acetyltransferase, or a peroxisome biogenesis factor.

45. The cell of claim 40, wherein the at least one gene is selected from one or more of the group consisting of POX1, POX2, POX3, POX4, POX5, POX6, BAT1, BAT2, YAT1, FAA1, ANT1, POT1, ACD1, IVD1, MFE1, PEX10, ARO10, GCDH1, GCDH2, ACD2, BCKD-E1α, BCKD-E1β, BCKD-E2, YALI0_D15708g, YALI0_B10406g, YALI0_D01265g, YALI0_F19910g, YALI0_D08690g, YALI0_F05038g, YALI0_D23815g, YALI0_D20768g, YALI0_D06930g, YALI0_E32835g, YALI0_F10857g, YALI0_D24750g, YALI0_E27654g, YALI0_C23859g, YALI0_E06567g, YALI0_C16797g, YALI0_F23749g, YALI0_B04906g, YALI0_D02387g, YALI0_E15378g, YALI0_E18568g, YALI0_D17864g, YALI0_E03058g, YALI0_F21197g, YALI0_E12573g, YALI0_F28567g, YALI0_F22121g, YALI0_D00671g, YALI0_D06215g, YALI0_D09383g, YALI0_D09493g, YALI0_A07733g, homologues thereof; and orthologues thereof.

46. The cell of claim 40, wherein the engineered cell expresses two or more enzymes of the cannabinoid biosynthesis pathway selected from the group consisting of a cannabigerolic acid (CBGA) synthase, an unsaturated cannabigerolic acid (CBGA-uns) synthase, a ketolated cannabigerolic acid (CBGA-keto) synthase, a cannabigerol(iso)butyric acid (CBG(i)BA) synthase, a cannabigerovarinic acid (CBGVA) synthase, a tetrahydrocannabinoic acid (THCA) synthase, an unsaturated tetrahydrocannabinoic acid (THCA-uns) synthase, a ketolated tetrahydrocannabinoic acid (THCA-keto) synthase, a tetrahydrocannabi(iso)butolic acid (THC(i)BA) synthase, a tetrahydrocannabivarinic acid (THCVA) synthase, a cannabichromenic acid (CBCA) synthase, an unsaturated cannabichromenic acid (CBCA-uns) synthase, a ketolated cannabichromenic acid (CBCA-keto) synthase, a cannabichromene(iso)butyric acid (CBC(i)BA) synthase, a cannabichromvarinic acid (CBCVA) synthase, a cannabidiolic acid (CBDA) synthase, an unsaturated cannabidiolic acid (CBDA-uns) synthase, a ketolated cannabidiolic acid (CBDA-keto) synthase, a cannabi(iso)butolic acid (CBD(i)BA) synthase, a cannabidivarinic acid (CBDVA) synthase, a hexanoyl-CoA synthetase, a (iso)valeryl-CoA synthetase, a butyryl-CoA synthetase, a polyketide synthase, a polyketide cyclase, a geranyl pyrophosphate (GPP) synthase, a HMG-CoA synthase, a mevalonate kinase, an acetyl-CoA synthase, a acetyl-CoA carboxylase, a acetyl-CoA acetyltransferase/HMG-CoA reductase, a mevalonate-5-phosphate decarboxylase, an isopentenyl phosphokinase, and a hydroxymethylglutaryl-CoA reductase.

47. The cell of claim 46, wherein the cell is capable of producing one or more cannabinoid derivatives selected from the group consisting of CBGA, CBGA-uns, CBGA-keto, CBGBA, CBGiBA, CBGVA, THCA, THCA-uns, THCA-keto, THCBA, THCiBA, THCVA, CBCA, CBCA-uns, CBCA-keto, CBCBA, CBCiBA, CBCVA, CBDA, CBDA-uns, CBDA-keto, CBDBA, CBDiBA, CBDVA, and decarboxylated derivatives thereof, when cultured under suitable conditions, optionally, wherein the suitable conditions comprise supplementation with one or more of butyric acid, butyl-butyrate, valeric acid, isovaleric acid, hexanoic acid, hexyl-hexanoate, glucose, glycerol, hexanol, butanol, and/or oleic acid.

48. The cell of claim 45, wherein the at least one gene comprises:
a) the combination of POX3, POX5 and one or both of ACD1 and IVD1, and wherein the cell demonstrates synergistic production of cannabinoid compared to a production of cannabinoid achieved with a cell comprising a deletion, inactivation or mutation of only ACD1, IVD1 or the combination of POX3/POX5;
b) the combination of YALI0_D15708g and YALI0_E12573g;
c) the combination of POX3, POX5, ACD1 and one or more of POX2, POX6, GCDH2, GCDH1, MFE1, YAT1, FAA1, ANT1, POT1, ACD2, and PEX10, and wherein the cell demonstrates an enhanced production of cannabinoid compared to a production of cannabinoid achieved with a cell comprising a deletion, inactivation or mutation of only the combination of POX3, POX5 and ACD1;
d) the combination of IVD1 and at least one of BAT1 or BAT2, and wherein the cell demonstrates an enhanced production of one or more C5 branched-chain cannabinoid compared to a production of the same one or more C5 branched-chain cannabinoid achieved with a cell comprising a deletion, inactivation, or mutation of only IVD1, optionally, wherein the one or more C5 branched-chain cannabinoid comprises one or more of CBGiBA, CBDiBA, CBCiBA, and/or THCiBA and their decarboxylated derivatives;
e) the combination of at least two of POX3, POX5 and ACD1;
f) the combination of POX3, POX5, ACD1 and FAA1, and wherein the cell demonstrates a significantly improved production of C4 cannabinoids compared to a production of C4 cannabinoid achieved with a cell comprising a deletion, inactivation, or mutation of only POX3, POX5, and ACD1;
g) the combination of POX3, POX5, ACD1 and YAT1, and wherein the cell demonstrates a significantly improved production of cannabinoids compared to a production of cannabinoid achieved with a cell comprising a deletion, inactivation, or mutation of only POX3, POX5, and ACD1; or
h) the combination of IVD1, one or both of BCKD-E1α and BCKD-E2, and optionally at least one of POX3, POX5, ACD1, and FAA1, and wherein the cell demonstrates a reduced production of C5 branched cannabinoid product, compared to a production of C5 branched cannabinoids achieved with a cell comprising a deletion, inactivation or mutation of only IVD1, optionally, wherein the cell additionally demonstrates a significant increased production of CBGVA compared to a production of CBGVA achieved with a cell which comprises a deletion, inactivation or mutation of only IVD1.

49. The cell of claim 40, wherein the at least one gene comprises FAA1, ACD1, IVD1, POX3, POX5, and one or both of BCKD-E1α and BCKD-E2, and wherein the cell demonstrates a significantly improved production of C4 cannabinoids compared to a production of cannabinoid achieved with a cell comprising a deletion, inactivation, or mutation of only POX3, POX5, ACD1, IVD1 and BCKD-E1α.

50. The cell of claim 40, wherein the at least one gene comprises FAA1, and optionally, wherein the at least one gene further comprises ACD1 or IVD1.

51. The cell of claim 40, wherein the cell is a yeast cell or a bacterial cell, optionally, wherein the yeast cell is a Yarrowia strain or a Saccharomyces strain.

52. A method of producing a cannabinoid or derivative thereof comprising culturing the cell of claim 40 under suitable conditions to produce the cannabinoid or derivative thereof, optionally, wherein the suitable condition comprises supplementing a culture media in which the cell is cultured with at least one of butyric acid, valeric acid, isovaleric acid, hexanoic acid, hexanol, butanol, oleic acid, glycerol or glucose.

53. The method according to claim 52, wherein the cannabinoid or derivative thereof comprises one or more of CBGA, CBGA-uns, CBGA-keto, CBGBA, CBGiBA, CBGVA, THCA, THCA-uns, THCA-keto, THCBA, THCiBA, THCVA, CBCA, CBCA-uns, CBCA-keto, CBCBA, CBCiBA, CBCVA, CBDA, CBDA-uns, CBDA-keto, CBDBA, CBDiBA, CBDVA, and decarboxylated derivatives thereof.

54. The method according to claim 52, further comprising the step of isolating the cannabinoid or derivative thereof from the culture.

55. A cell engineered to produce one or more of CBGiBA, CBGA-uns, CBGA-keto, THCiBA, THCA-uns, THCA-keto, CBCiBA. CBCA-uns, CBCA-keto, CBDiBA, CBD-uns, CBD-keto, and/or decarboxylated derivatives thereof, wherein the cell comprises an inactivated or otherwise mutated IVD1, POT1, and/or MFE1 gene, and wherein the cell further expresses sufficient enzymes in the cannabinoid synthesis pathway to produce the one or more of CBGiBA, CBGA-uns, CBGA-keto, THCiBA, THCA-uns, THCA-keto, CBCiBA. CBCA-uns, CBCA-keto, CBDiBA, CBD-uns, CBD-keto, and/or decarboxylated derivatives thereof, when cultured under suitable conditions, optionally, wherein the suitable conditions comprise supplementation of a culture media in which the cell is cultured with butyric acid, butyl-butyrate, valeric acid, isovaleric acid, butanol, hexanol, hexanoic acid, hexyl-hexanoate, oleic acid, glycerol or glucose.

56. A method of producing one or more of CBGiBA, CBGA-uns, CBGA-keto, THCiBA, THCA-uns, THCA-keto, CBCiBA, CBCA-uns, CBCA-keto, CBDiBA, CBD-uns, CBD-keto, and/or decarboxylated derivatives thereof, by culturing the cell of claim 55 under conditions suitable to produce the one or more of CBGiBA, CBGA-uns, CBGA-keto, THCiBA, THCA-uns, THCA-keto, CBCiBA. CBCA-uns, CBCA-keto, CBDiBA, CBD-uns, CBD-keto, and/or decarboxylated derivatives thereof, optionally, wherein the suitable conditions comprise supplementing a culture media in which the cell is cultured with at least one of butyric acid, butyl-butyrate, valeric acid, isovaleric acid, hexanoic acid, hexyl-hexanoate, hexanol, butanol, oleic acid, glycerol or glucose.

57. The method of claim 56, further comprising the step of isolating the one or more of CBGiBA, CBGA-uns, CBGA-keto, THCiBA, THCA-uns, THCA-keto, CBCiBA. CBCA-uns, CBCA-keto, CBDiBA, CBD-uns, CBD-keto, and/or decarboxylated derivatives thereof from the culture.

58. A compound according to any of the following structures: 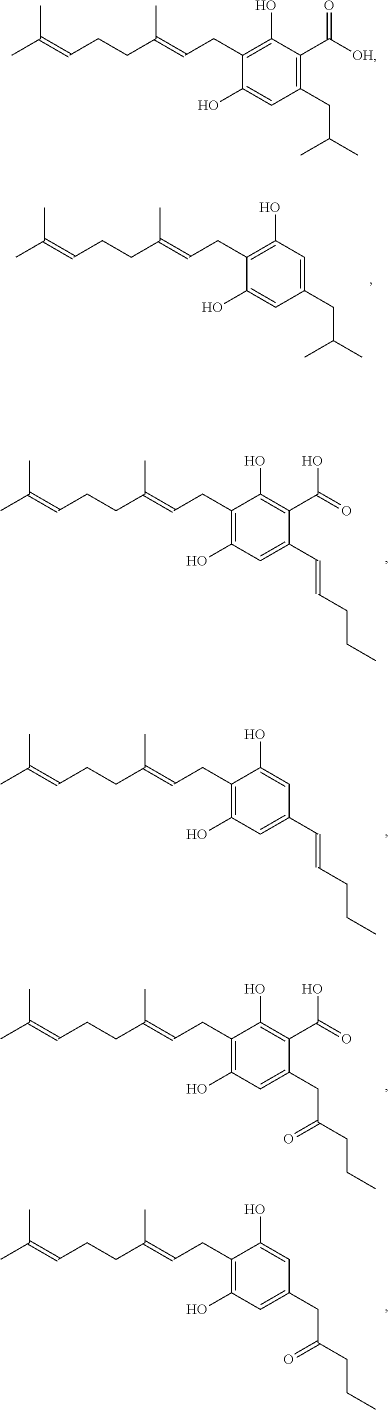 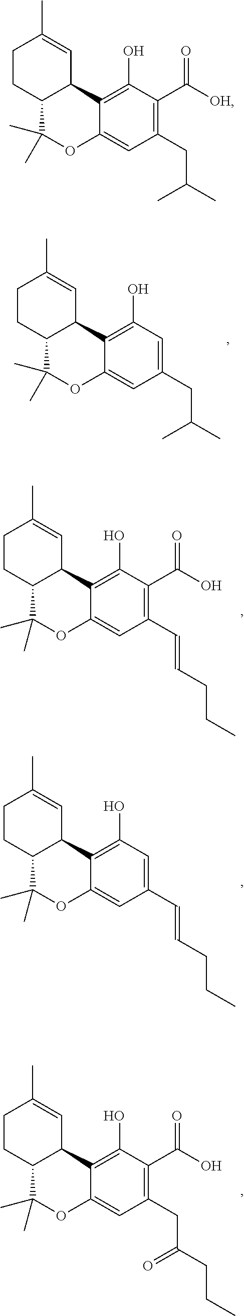 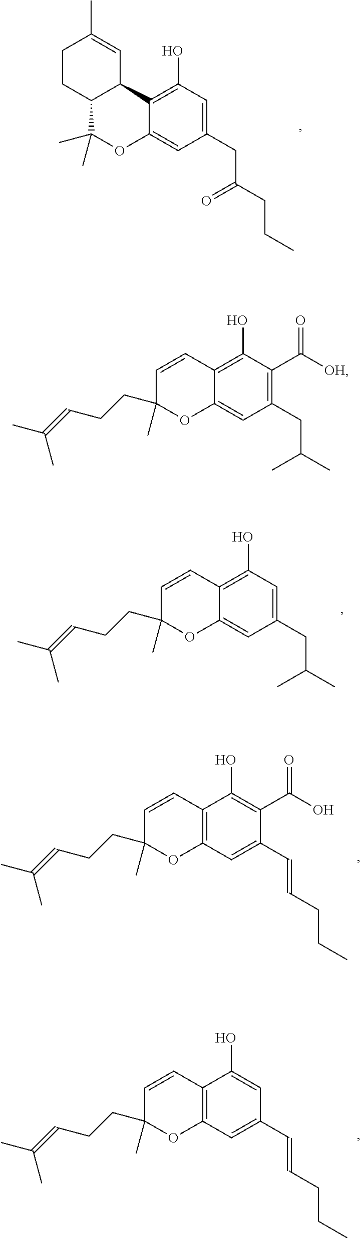 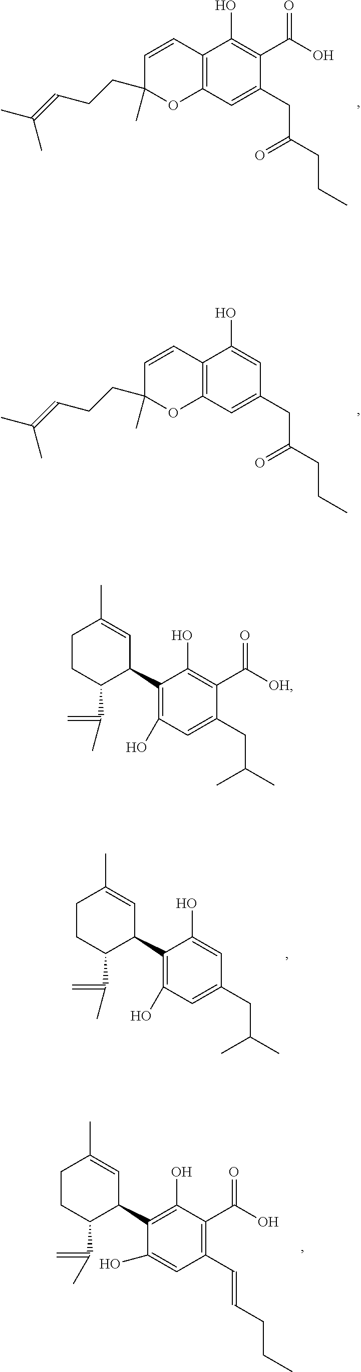 [chemical structure]
